(19) United States
(12) Patent Application Publication
(10) Pub. No.: US 2025/0281573 A1
(43) Pub. Date: Sep. 11, 2025

(54) Methods of Treating Retinal Degenerative Diseases Using AIMP2-DX2 and Optionally a Target Sequence for miR-142 and Compositions Thereof

(71) Applicant: Generoath Co., Ltd., Seoul (KR)

(72) Inventors: Jin Woo CHOI, Seoul (KR); Kyunghwa BAEK, Gangneung-si, Gangwon-do (KR)

(21) Appl. No.: 18/858,540

(22) PCT Filed: May 13, 2023
(86) PCT No.: PCT/IB2023/054956

§ 371 Date: Oct. 21, 2024

Related U.S. Application Data
(60) Provisional application No. 63/341,798, filed on May 13, 2022.

(51) Int. Cl. A61K 38/17; A61K 38/18; A61K 39/395; A61K 48/00; A61P 27/02

(52) U.S. Cl. CPC A61K 38/1709; A61K 38/1866; A61K 39/3955; A61K 48/0033; A61K 48/0066; A61P 27/02

(57) ABSTRACT

Disclosed herein are methods of treating retinal degenerative diseases, comprising administering to a subject in need thereof a vector comprising AIMP2-DX2 and optionally a target sequence for miR-142.

### REFERENCE TO SEQUENCE LISTING SUBMITTED ELECTRONICALLY

The content of the electronically submitted sequence listing in ST26 file (Name: 2493-0006 WO01.xml_Sequence Listing_ST25.txt; Size: 36 KB; and Date of Creation: May 8, 2023) filed with the application is incorporated herein by reference in its entirety.

### FIELD OF THE INVENTION

Disclosed herein are methods of treating retinal degenerative diseases, comprising administering to subjects in need thereof a vector comprising AIMP2-DX2 and optionally a target sequence for miR-142.

### BACKGROUND OF THE INVENTION

Retinal degenerative diseases are characterized by the degeneration of one or two retinal cell types, such as retinal pigment epithelial (RPE) and/or photoreceptor cell loss in age-related macular disease (AMD) and retinitis pigmentosa (RP), and retinal ganglion cell (RGC) death in glaucoma. Typically, visual impairment caused by degenerating retinal cells is irreversible because retinal cells lack self-repair capability.

Aberrant RPE cells can be observed through optical coherence tomography (OCT) in AMD patients. In retinitis pigmentosa (RP) patients, the morphological and functional abnormalities of RPE and photoreceptor layers are caused by a genetic abnormality. Stargardt's disease or juvenile macular degeneration, which is characterized by the loss of the RPE and photoreceptors in the macular area, causes central vision loss at an early age. Loss of retinal ganglion cells (RGCs) can be observed in patients with glaucoma. Once the retinal cell degeneration is triggered, no treatments can reverse it.

In the retina, light signals are transformed into electrical signals. Various interconnected cell types, such as photoreceptor cells, bipolar cells, horizontal cells, amacrine cells, and retinal ganglion cells (RGCs), are required to accomplish this key process in the retina. Each cell type plays an indispensable role in the visual signaling process. Visual signaling starts in photoreceptor cells, which express photosensitive proteins mostly in the outer segments. Jin ZB et al. (2019).

In the human retina, two types of photoreceptor cells, rods and cones, are responsible for dim light vision and daylight vision (including color), respectively. Rods are located mainly in the peripheral retina, and cones are concentrated in a small portion of the retina, the macula, which provides high-resolution central vision. Bipolar cells receive visual signals from photoreceptor cells and transmit these signals to RGCs coordinated by horizontal and amacrine cells. Ultimately, all the signals are gathered in the optic nerve and transmitted to the brain. Both cell degeneration and synaptic disruption can cause permanent visual impairment or even blindness. See, Jin 2019.

Age-related macular disease (AMD) is the leading cause of vision loss in Europe, the United States, and Australia. Almost two-thirds of the population over 80 years of age will have signs of AMD resulting from the wet or exudative form, which is characterized by the presence of drusen and CNV (subfoveal choroidal neovascularization). AMD is a chronic progressive disease characterized by damage to the central retina zone. Changes in choriocapillaries, retinal pigment epithelium (RPE), and Bruch's membrane (typical for aging) underlie AMD pathogenesis; however, the mechanisms launching the transfer of typical age-related changes in the pathological process are unknown. The death of photoreceptors and irreversible loss of vision become the results of pathological changes in RPE and choroid. Recent studies demonstrated that not only apoptotic but also autophagic and necrotic signaling cascades are involved in the cellular death of retinal cells (Telegina 2017).

“Dry” and “wet” forms of the disease are categorized. Approximately 90% of cases are of the “dry” atrophic form of AMD; today, there is no method for its treatment. In the “dry” form of AMD, drusen are diagnosed in the macular area, pigment redistribution occurs, defects of pigment epithelium and the choriocapillary layer appear, and death of photoreceptors occurs against a background of RPE cell atrophy. “Wet” (exudative) form develops in ˜10% of AMD patients and is characterized by ingrowing of newly generated vessels through Bruch's membrane defects under the retinal pigment epithelium or under neuroepithelium. The increased permeability of newly generated vessels results in retinal edema, exudation, and hemorrhage in the vitreous body and retina (which finally becomes the reason for vision loss).

Currently, the laser-induced Bruch's membrane photocoagulation model is the most widely accepted and most frequently used experimental mouse CNV model. The model described here consists of the laser impact rupturing of Bruch's membrane, which leads to the growth of new blood vessels from the choroid into the subretinal space, mimicking the main characteristics of the exudative form of human AMD and offering the opportunity to explore the molecular mechanism of CNV through the use of a large panel of transgenic mice. The model has proven to be suitable for testing the efficacy of new drugs through systemic or local (intraocular) administration and has shown predictive value for drug effects in patients with AMD, for example, with vascular endothelial growth factor receptor (VEGFR) trap or anecortave acetate (Telegina 2017).

There are three main drugs that provide indirect anti-angiogenesis by blocking vascular endothelial growth factor (VEGF) in the retina. Ranibizumab (Lucentis, Genentech Inc., South San Francisco CA; commercialized worldwide by Novartis) was approved by the Food and Drug Administration (FDA) in 2006 for the treatment of neovascular AMD (Hernández-Zimbrón 2018). It is a recombinant humanized Immunoglobulin (Ig) G1 kappa isotype monoclonal antigen-binding fragment (Fab) that targets and binds VEGF-A with high affinity. Aflibercept (Regeneron, Tarrytown, NY; commercialized worldwide by Bayer AG) was approved by the FDA in 2011. It is a fusion protein that combines two key binding domains of human VEGF receptors 1 and 2 and a fragment crystallizable (Fc) region of a human IgG1. Finally, bevacizumab (Genentech Inc., South San Francisco, CA; commercialized worldwide by Roche) is a full-length humanized antibody that binds and blocks all VEGF isoforms.

Oxidative stress can trigger apoptosis, which may activate and recruit macrophages and induce inflammation. Apoptosis may be one of the triggers of choroidal inflammation and consequently angiogenesis in CNV model (Du 2013).

The most common form of recessive RP is X-linked RP, which is caused by mutations in the retinitis pigmentosa GTPase regulator (RPGR) gene. Talib M et al. (2018). X-linked RP is characterized by the degeneration of rod and cone photoreceptors during childhood, leading to visual field constriction and severe sight loss at an early age. Despite promising results in animal models (Bennett J (2017); Boye S E et al. (2013); Sahel J A et al. (2014); Vandenberghe LH (2015)), gene therapy targeting photoreceptor degeneration in humans for RPGR related RP has not been fully developed until now. Gene therapy using an adeno-associated viral vector is considered the most promising approach for treating this condition (Megaw R D et al. (2015)), although the RPGR coding sequence's inherent instability poses a challenge in translating this therapy into human trials.

The approval of gene therapy targeting RPE65-related retinal degeneration is a major breakthrough in clinical gene therapy application and has conferred a potential to treat numerous genetic diseases. Russell S et al. (2017). However, the RPGR coding sequence has inherent instability, which contains a repetitive purine-rich region and undergoes alternative splicing, posing challenges to the translation of this therapy into human trials. Deng W T et al. (2015); Pawlyk B S et al. (20016); Sun X et al. (2016); Wu Z et al. (2015).

Neurodegenerative disorders (NDD) represent a heterogeneous group of diseases characterized by progressive structural and functional degeneration of the central and peripheral nervous systems. Due to its special connection to the brain and its accessibility to measurements, the eye provides a unique window on the brain, thereby offering non-invasive access to a large set of potential biomarkers that might help in the early diagnosis and clinical care of NDD.

Aside from traditional age- and genetics-related vision conditions, such as macular degeneration, central nervous system conditions including Parkinson's disease and Alzheimer's disease can affect patients' vision. These diseases can cause direct or retrograde degenerations of the optic nerve, retinal cells, and surrounding visual structures. While not often obvious, these conditions affect coordination, mobility, and visual perception, resulting in increased risks of falls and related injuries.

AIMP2-DX2 is an alternative, antagonistic splicing variant of AIMP2, which is a multifactorial apoptotic gene. AIMP2-DX2 is known to suppress cell apoptosis by hindering the functions of AIMP2. AIMP2-DX2, acting as competitive inhibitor of AIMP2, suppresses TNF-alpha mediated apoptosis through inhibition of ubiquitination/degradation of TRAF2. In addition, it had been reported that AIMP2-DX2 has been confirmed as an existing lung cancer induction factor and, in the existing research, it was confirmed that AIMP2-DX2, manifested extensively in cancer cells, induces cancer by interfering with the cancer suppression functions of AIMP2. Moreover, it was discovered that manifestation of AIMP2-DX2 in normal cell progresses cancerization of cells while suppression of manifestation of AIMP2-DX2, suppresses cancer growth, thereby displaying treatment effects.

It has also been determined that AIMP2-DX2 can be useful in treating neuronal diseases (KR10-2015-0140723 (2017) and US2019/0298858 (2019).

### SUMMARY OF THE INVENTION

Disclosed herein are methods of treating a retinal degenerative disease in a subject in need thereof, comprising administering to the subject a pharmaceutically effective amount of a nucleic acid molecule comprising an exon 2-deleted AIMP2 variant (AIMP2-DX2 or DX2). In some embodiments, the nucleic acid molecule is a viral vector or a nonviral vector, e.g., a recombinant vector.

Also disclosed herein are uses of a pharmaceutically effective amount of a nucleic acid molecule comprising an exon 2-deleted AIMP2 variant (AIMP2-DX2 or DX2) for the treatment of a retinal degenerative disease in a subject in need thereof. Also disclosed herein are a pharmaceutically effective amount of a nucleic acid molecule comprising AIMP2-DX2 for uses in the treatment of a retinal degenerative disease in a subject in need thereof. Also disclosed herein are the uses of a pharmaceutically effective amount of a nucleic acid molecule comprising AIMP2-DX2 for the manufacture of medicaments for treatment of a retinal degenerative disease in a subject in need thereof.

Disclosed herein are methods of treating retinal degenerative diseases in a subject in need thereof, comprising administering to the subject a pharmaceutically effective amount of a recombinant vector comprising an exon 2-deleted AIMP2 variant (AIMP2-DX2 or DX2) gene.

Also disclosed herein are uses of a pharmaceutically effective amount of a recombinant vector comprising an exon 2-deleted AIMP2 variant (AIMP2-DX2 or DX2) for the treatment of a retinal degenerative disease in a subject in need thereof. Also disclosed herein are a pharmaceutically effective amount of a recombinant vector comprising AIMP2-DX2 for uses in the treatment of a retinal degenerative disease in a subject in need thereof. Also disclosed herein are the uses of a pharmaceutically effective amount of a recombinant vector comprising AIMP2-DX2 for the manufacture of medicaments for treatment of a retinal degenerative disease in a subject in need thereof.

In some embodiments, the nucleic acid molecule is administered by a chemical conjugation with Galnac, a cell-penetrating peptide, a nucleic acid or glycopeptide mediated tool, a synthetic compound vector; a lipid-mediated delivery such as lipid nanoparticle encapsulation; an inorganic vector mediated delivery such as inorganic vectors; a biological delivery such as exosome mediated delivery; or a physical delivery such as by micro/nano needles, pressure-perfusion, microprojectiles, electrical energy/electroporation/iontophoresis, sonoporation, magnetoporation, or optoporation/photodynamic energy.

In some embodiments, the retinal degenerative disease is retinitis pigmentosa, Leber's congenital amaurosis, Cone-rod dystrophy, glaucoma, or diabetic retinopathy. In some embodiments, the retinal degenerative disease precedes or is accompanied by Parkinson's disease, Alzheimers's disease, or amyotrophic lateral sclerosis. In some embodiments, the retinal degenerative disease is not age-related macular disease. In some embodiments, the methods of treating retinal degenerative diseases does not include treating age-related macular disease (AMD). In some embodiments, the AMD is wet AMD. In some embodiments, the AMD is dry AMD. The recombinant vector can further comprise an miR-142 target sequence.

The vector can further comprise a promoter operably linked to the AIMP2-DX2. In some embodiments, the promoter is a Retrovirus (LTR) promoter, cytomegalovirus (CMV) promoter, Rous sarcoma virus (RSV) promoter, MT promoter, EF-1 alpha promoter, UB6 promoter, chicken beta-actin promoter, CAG promoter, RPE65 promoter or opsin promoter.

The miR-142 target sequence can be 3′ to the AIMP2-DX2 gene.

In some embodiments, the AIMP2-DX2 gene comprises a nucleotide sequence encoding an amino acid sequence that is at least 90% identical to SEQ ID NO:2, 13, 14, 15, 16, 17, 18, 19, or 20.

In some embodiments, the AIMP2-DX2 gene comprises a nucleotide sequence encoding an amino acid sequence of SEQ ID NO:2, 13, 14, 15, 16, 17, 18, 19, or 20.

In some embodiments, the AIMP2-DX2 gene does not have an exon comprising a nucleotide sequence encoding an amino acid sequence that is at least 90% identical to SEQ ID NO: 10 or 11.

In some embodiments, the AIMP2-DX2 gene does not have an exon comprising a nucleotide sequence encoding an amino acid sequence of SEQ ID NO:10 or 11.

The miR-142 target sequence can comprise a nucleotide sequence comprising ACACTA. In some embodiments, the miR-142 target sequence comprises ACACTA and 1-17 additional contiguous nucleotides of SEQ ID NO:5. In some embodiments, the miR-142 target sequence comprises a nucleotide sequence at least 50% identical to a nucleotide sequence of SEQ ID NO:5 (TCCATAAAGTAGGAAACACTACA; miR-142-3p target sequence). In some embodiments, the miR-142 target sequence can comprise a nucleotide sequence of SEQ ID NO:5.

In some embodiments, the miR-142 target sequence comprises ACTTTA. In some embodiments, the miR-142 target sequence comprises ACTTTA and 1-15 additional contiguous nucleotides of SEQ ID NO:7. In some embodiments, the miR-142 target sequence comprises a nucleotide sequence at least 50% identical to a nucleotide sequence of SEQ ID NO:7 (AGTAGTGCTTTCTACTTTATG; miR-142-5p target sequence). In some embodiments, the miR-142 target sequence comprises a nucleotide sequence of SEQ ID NO:7.

The miR-142 target sequence can be repeated 2-10 times in the vector disclosed herein.

The vector can be a viral vector. The viral vector can be an adenovirus, adeno-associated virus, lentivirus, retrovirus, human immunodeficiency virus (HIV), murine leukemia virus (MLV), avian sarcoma/leukosis (ASLV), spleen necrosis virus (SNV), Rous sarcoma virus (RSV), mouse mammary tumor virus (MMTV), Herpes simplex virus, or vaccinia virus vector.

In some embodiments, the recombinant vector is administered topically to, by intravitreal injection to, by subconjunctival injection to, or into a subretinal space of the subject.

The methods disclosed herein can further comprise administering to the subject an additional therapeutic agent. In some embodiments, the additional therapeutic agent is ranibizumab, aflibercept, or bevacizumab.

### BRIEF DESCRIPTION OF THE FIGURES

FIG. 1. An example recombinant vector.

FIG. 2. Nerve cell-specific expression of a recombinant vector under an in vitro environment.

FIG. 3. An miR142-3 pT (target) sequence with 4 repeats of miR142-3 pT (underlined) (SEQ ID NO:6).

FIG. 4A. A schematic of miR142-3 pT with 1×, 2×, and 3× repeats, and mutant. FIG. 4B shows miR142-3p inhibition on DX2 expression with 1×, 2×, and 3× repeats of miR-142-3 pT.

FIG. 5. A core binding sequence is important in DX2 inhibition. A vector with Tseq x3 repeats, which showed significant inhibition of DX2 (FIG. 4B), and DX2 construct were used as controls. 100 pmol of miR-142-3p treatment inhibited Tseq x3 vector significantly but DX2 and mutant sequence were not inhibited.

FIGS. 6A-6C. A comparison of the amino acid sequences of AIMP2-DX2 and variants (FIGS. 6B and 6C are continuations of FIG. 6A).

FIGS. 7A-7I. DX2 physically binds with AIMP2 to decrease PARP-1 activation. FIG. 7A. AIMP2, and DX2 expression was induced by transfection of each plasmid in SH-SY5Y cells and then followed by analyses of PARP-1 pull-down assays. DX2 shows a higher affinity for PARP-1 than AIMP2. FIG. 7B. SH-SY5Y cells were transfected with vehicle (Con) or DX2 expression (DX2) plasmid, incubated for 24 hours and lysed. Total cell lysates were incubated with protein agarose beads to immunoprecipitate PARP-1 bound AIMP2 and analyzed by immunoblot analysis. FIG. 7C. DX2 siRNA (DX2) and control siRNA (Con) were transfected into SH-SY5Y and incubated for 48 hours. Total cell lysates were incubated with protein agarose beads to immunoprecipitate PARP-1 bound AIMP2 and analyzed by immunoblot analysis. FIGS. 7D and 7E. GFP- or Myc-tagged AIMP2 and/or DX2 expressing plasmid was transfected into HEK 293 cells and the binding affinity was measured by immunoprecipitation (IP) with myc antibody (FIG. 7D) and GFP antibody (FIG. 7E). FIGS. 7F and 7G. SH-SY5Y cells were transfected with the EV (empty vector), AIMP2 and DX2 expression plasmid, and 24 hours later, the transfected cells were incubated with or without 10 400 μM H2O2 for 4 hours. Cleaved PARP-1 levels (FIG. 7F) and PARlyation (FIG. 7G) were examined using immunoblot assays. In oxidative stressed-induce cellular damage conditions, DX2 attenuates cleavage of PARP-1 (FIG. 7F) and PARylation (FIG. 7G) related to cell death. FIG. 7H. AIMP2 and DX2 protein stability were assessed with cycloheximide-treated pulse chase assay. The samples were harvested in the time-dependent manner 0 hour to 4 hours and examined using immunoblot assays. FIG. 7I. Schematic figure of AIMP2-induced PARP-1 activation. In the absence of DX2, AIMP2 dimer induces PARP-1 activation and neuronal death. However, in the presence of DX2, DX2 interacts with AIMP2 and inhibits PARP-1 activation.

FIGS. 8A-8C. DX2 reduces neurotoxin-induced neuronal cell death. FIG. 8A. N2A cells were transfected with EV, AIMP2 and DX2 expression plasmids and the transfected cells were incubated with or without H₂O₂ for 4 hours, then the cell viability was measured using MTT. FIGS. 8B and 8C. SH-SY5Y cells were co-transfected with EV, AIMP2 and DX2 expression plasmids, incubated with H₂O₂ for 4 hours and the cell viability was measured by MTT analysis. The number shows the amounts of transfected DNA.

FIGS. 9A-9C. DX2 compromises pro-apoptotic activity of AIMP2 via the competitive inhibition of AIMP2 binding to TRAF2. FIG. 9A. HEK293 cells were transfected with the indicated amount of Myc-DX2 and -AIMP2. The expression of endo- and exo-AIMP2 and -DX2 was confirmed by western blotting of whole cell lysates (WCL) with anti-AIMP2 antibody (lower panel). TRAF2 was immunoprecipitated with its specific antibody and AIMP2 or DX2 bound to TRAF2 were detected with anti-Myc antibody (upper panel). Choi J W et al. (2012). FIG. 9B. DX2 increased the NF-kB activity, whereas AIMP2 gave the opposite effect. FIG. 9C. DX2 overexpression abolished the TNF-α induced cell death.

FIGS. 10A-10B. Overexpression of DX2 reduces TNFa-induced neuronal cell death. FIG. 11A. N2A cells were transfected with EV and DX2 and treated with TNF-α and CHX for 6 h. Cell viability was evaluated by the MTT assay. #P<0.05. FIG. 10B. Primary neuronal cells in WT mice were transfected with EV (empty vector) and DX2, and the MTT assay was performed to assess their viability. #P<0.0.

FIGS. 11A-11B. AAV2-GFP transduction efficacy test. FIG. 11A. Transduction efficacy test of AAV-GFP. The SK-SY5Y cells were infected with scAAV-GFP or ssAAV-GFP, and 48 hours later, (FIG. 11B) GFP expression was observed on fluorescence microscopy.

FIGS. 12A-12G. AAV2-DX2 has anti-apoptotic effects and alters cellular signaling. FIG. 12A. Cytotoxic effects in the primary neurons (Neuron), MEF (Mouse embryonic fibroblasts), hepatocytes, and MSC (Mesenchymal stem cells). After H₂O₂ treatment, a decreased cytotoxic effect was observed in DX2 transduced cells (AAV2-DX2) when compared with control-transduced cells (c). FIG. 12B. DX2 is not required for normal cell growth in SH-SY5Y (left) and primary neuronal cells (right). In oxidative stress conditions, AAV2-DX2-infected cells show decreased levels of cell death when compared to their control counterparts (AAV-GFP, GFP). FIG. 12C. Enrichment plot of RNAseq of AAV-GFP or AAV2-DX2-infected neuroblastoma cells SK-N-SH and N2A. FIGS. 12D and 12E. Graph of gene counts representing cell death and inflammatory related pathways were downregulated. FIGS. 12F and 12G. p-value plot of the signaling pathway changed by DX2 overexpression. ns; non-significant; * P<0.05; **, P<0.01; ****, P<0.0001, t-test.

FIG. 13. DX2 increased retinal photoreceptor neuronal cell viability. 661 W cells were seeded at 5×10\hat{ }4 cells per well in a 96-well plate, followed by infection with AAV-DX2 virus at an 5000MOI. After 48 hours, cells were treated with varying concentrations of H2O2. Cell viability was assessed 24 hours later using the MTT assay.

FIG. 14. DX2 reduces the levels of BAX induced by H2O2. 661 W cells were infected with either AAV-GFP or AAV-DX2 at an MOI of 5,000 each. After 72 hours, cells were treated with 2 mM H₂O₂ for 6 hours, and then the RNA levels of BAX were assessed.

FIGS. 15A-15B. Overexpression of DX2 results in an increase in 67LR levels in 661 W cells. FIG. 15A. Empty vector (EV) and Strep-DX2 (DX2) were transfected into 661 W cells using Lipofectamine 3000 reagent. DX2 transfection resulted in an increase in 67LR levels. FIG. 15B. Immunofluorescence staining was performed on 661 W cells under the same conditions as in FIG. 15A.

FIG. 16. DX2 binds to KRS similarly to AIMP2. Cells transfected with 2 μg of Flag-tag-AIMP2 and Flag-tag-DX2 were subjected to cell lysis using NP-40 lysis buffer. Subsequently, a mixture of 15 μl agarose A/G plus beads and KRS antibody was used for the pull-down assay.

FIGS. 17A-17B. DX2 modulates the levels of KRS membrane form under ROS conditions, leading to an increase in 67LR levels. FIG. 17A. 661 W cells were infected with AAV-GFP and AAV-DX2 at a multiplicity of infection (MOI) of 5,000, followed by treatment with 2 mM H₂O₂ for 6 hours after 72 hours post-infection. Subsequently, cell lysis was performed and changes in KRS and 67LR levels were observed using Western blot analysis. FIG. 17B. Following the same experimental conditions as in FIG. 17A, cells were fractionated into cytosolic and membrane components, and Western blot analysis was conducted. Upon H₂O₂ treatment, a reduction in the membrane form of KRS was observed, while treatment with AAV-DX2 resulted in an increase in the membrane forms of both 67LR and KRS.

FIG. 18A. Cross-sectional histology (H&E staining) of retina. FIG. 18B. Retina thickness. FIG. 18C. RPE (Retinal Pigment Epithelial) thickness. FIG. 18D. ONL (Outer Nuclear Layer of Photoreceptors) thickness. FIG. 18E. Outer Segment thickness. FIG. 18F. OPL Outer Plexiform Layer) thickness. All the samples were acquired from the optic nerve containing section with 10 μm thickness. FIG. 18G. Integrity and proliferation of RPE (Retinal Pigment Epithelial). FIG. 18H. PR (Photoreceptor) recovery. FIG. 18I. Cellular proliferation of RPE and PR. FIG. 18J-180. Functional recovery of retina. Electroretinograph of normal ERG graph format (FIG. 18J-18K). A-wave amplitude (FIG. 18L) and A-wave latency (FIG. 18M), b-wave amplitude (FIG. 18N) and b-wave latency (FIG. 18O).

FIG. 19A. Fluorescein angiography and indocyanine green angiography images of laser photocoagulation sites after treatment with scAAV2-GFP or scAAV2-DX2. FIGS. 19B-19D. Comparison of choroidal neovascularization area. The ratios of leaky area to CNV area when treated with GFP control and DX2. The leaky area was estimated by measuring the total hyperfluorescent area using FA, and the CNV area was calculated using ICGA. Change in CNV area by treatment with DX2. The CNV volume was calculated based on the total measured Isolectin B4 volume. For each group, n=12, *P< (0.05). FIG. 19E. Expression of VEGF in laser-induced choroidal neovascularization (CNV) mouse model. Western blot result of VEGF expression and VEGF fold change in CNV mouse model. VEGF fold change was measured by Image J. For each group, n=6, *P< (0.05).

FIGS. 20A-20B. Retinal pigment epithelium (RPE) degenerated area Fundus photography. FIGS. 20C-20F. The DX2 expression level of test articles (AAV2-DX2) treated groups. FIGS. 20G-20J. Immunohistochemistry. FIGS. 20K-20L. Electroretinogram.

FIGS. 21A-21B. Electroretinogram measurements of A wave (FIG. 21A) and B wave (FIG. 21B).

FIGS. 22A-22B. AAV2-DX2AAV2-DX2 prevents neovascularization in Laser-induced choroidal neovascularization (CNV) rabbit model. FIG. 22A. The fluorescein intensity. FIG. 22B. Isolectin B4 staining.

FIGS. 23A-23M. Biodistribution study to assess DX2 expression in whole-body organs. FIGS. 23A. qPCR analysis to confirm expression of DX2 in injected tissue area. FIGS. 23B-23M. Analysis of DX2 expression in thymus, liver, testis, brain, spleen, kidney, heart, stomach, mesenteric lymph node, spinal cord, pancreas, and lung using qPCR.

### DETAILED DESCRIPTION OF THE INVENTION

Neurodegenerative disorders (NDD) represent a heterogeneous group of diseases characterized by progressive structural and functional degeneration of the central and peripheral nervous systems. Due to its special connection to the brain and its accessibility to measurements, the eye provides a unique window on the brain, thereby offering non-invasive access to a large set of potential biomarkers that might help in the early diagnosis and clinical care of NDD.

Inherited retinal disease, especially RP or Leber congenital Amurosis, is a collective concept that refers to heterogeneous eye diseases that are caused by dozens of mutations in various genes and vary in presence and frequency depending on race and region. Recently, gene therapy to replace a mutated gene with normal WT gene has emerged as a promising treatment method. However, most of the gene therapy drugs that are currently undergoing clinical phase studies are therapy through the introduction of a single normal WT gene to replace the mutant gene, which is a limit that can only be expected to be effective in a very limited number of patients. In addition, the clinical study outcome of single mutant gene replacement gene therapy (e.g., Luxturna) so far is still far from normal vision recovery. Only efficacy to enhance light detection ability to enable a patient's daily life on their own has been reported. This suggests that there are more complex cell-level mechanisms in normal vision recovery in addition to single mutant gene replacement.

Aside from traditional age- and genetics-related vision conditions, such as macular degeneration, central nervous system conditions including Parkinson's disease and Alzheimer's disease can affect patients' vision. These diseases can cause direct or retrograde degenerations of the optic nerve, retinal cells, and surrounding visual structures. While not often obvious, these conditions affect coordination, mobility, and visual perception, resulting in increased risks of falls and related injuries.

Parkinson's disease is a progressive neuronal degenerative disorder. Destruction of dopaminergic neurons in the substantia niagra with a consequent reduction of dopamine actions in the corpus striatum, parts of the basal ganglia system that are involved in motor control is thought to be a pathophysiologic mechanism of Parkinson's disease. While Parkinson's disease is primarily characterized by tremors, rigidity, and postural instability, vague visual symptoms such as blurred and double vision, uncontrolled eye movements, light sensitivity, eye strain, and difficulty reading are common. Mild ocular motor abnormalities occur in as many as 75 percent of patients with idiopathic Parkinson's disease but often are left untreated. Yet because these symptoms can further reduce a patient's already jeopardized quality of life and functionality, they warrant specialized treatment.

Amyotrophic lateral sclerosis (ALS) is a fatal neurodegenerative disease resulting in a gradual loss of motor neuron function. Although ophthalmic complaints are not presently considered a classic symptom of ALS, retinal changes such as thinning, axonal degeneration and inclusion bodies have been found in many patients. Retinal abnormalities observed in postmortem human tissues and animal models are similar to spinal cord changes in ALS. These findings are not dramatically unexpected because retina shares an ontogenetic relationship with the brain, and many genes are associated both with neurodegeneration and retinal diseases. See Soldatov 2021.

Alzheimer's disease is the most common form of dementia, and it is characterized by destroyed and damaged connections between neural cells. These injuries occur due to abnormal accumulations of β-amyloid protein (Abeta) in the form of amyloid plaques and/or neurofibrillary tangles composed of aggregated tau protein. Ocular degeneration in Alzheimer's disease occurs as the retinal nerve fiber layer thins. Neurons responsible for visual processing tend to be damaged more than primary vision neurons, resulting in ambiguous vision symptoms in the early stages of the disease. Deficits in recognizing objects, seeing colors, and processing visual motion can occur at any stage, increasing the risk of accidents such as falls, lacerations, and burns.

DX2, an exon 2-deficient antagonistic splicing variant of AIMP2, effectively compromises AIMP2-induced neuronal death. AIMP2 is one of three auxiliary proteins that form the multi-tRNA synthetase complex. Besides their role in supporting tRNA ligation during translation, AIMP2 has been reported to play as a multi-functional and potent cell death-inducing gene.

AIMP2 over-activates cell apoptotic signaling pathways such as PARP-1 or p53 or suppresses cell survival signaling pathways such as TRAF signaling or LR expression.

As a splicing variant of AIMP2, DX2 acts as an antagonistic competitor against AIMP2, consequently directly and indirectly inhibits AIMP2 induced cell death.

DX2 inhibits PARP-1 and p53-induced neuronal death in Parkinson's disease model. Overexpression of DX2 using AAV in the substantia nigra rescues motor activity and neuronal cell death in PD-induced mouse models.

In ALS, DX2 inhibits TRAF2 dependent and 67 LR mediated neuronal death in ALS disease. Overexpression of DX2 improved locomotive activity and survival and reduced neuronal cell death in the ALS mouse model.

Without being bound by any theory, given with the anatomical and developmental interconnection between eye and brain and the clinical evidence for retinal damage in NDD, there can be a common pathophysiological pathway in destruction of neuronal cell system in CNS and degenerations of the optic nerve, retinal cells, and surrounding visual structures.

Gene therapy is a promising treatment for irreversible retinal cell death in diverse diseases, such as age-related macular degeneration (AMD), Stargardt's disease, retinitis pigmentosa (RP) and glaucoma. Adeno-associated virus (AAV) has emerged as the preferred vector for targeting gene expression to the retina. Subretinally or by intravitreally injected AAV can efficiently transduce retinal pigment epithelium and inner layer of retina. AAV is also appreciated as a suitable gene delivery approach because of its relative safety due to a lack of pathogenicity (20). Utilization of survival gene DX2 can be a potential option for treatment of retinal degenerative diseases.

AIMP2-DX2 is an alternative, antagonistic splicing variant of AIMP2 (aminoacyl tRNA synthase complex-interacting multifunctional protein 2), which is a multifactorial apoptotic gene. AIMP2-DX2 is known to suppress cell apoptosis by hindering the functions of AIMP2.

AIMP2-DX2, acting as a competitive inhibitor of AIMP2, suppresses TNF-alpha mediated apoptosis through inhibition of ubiquitination/degradation of TRAF2.

It has also been determined that AIMP2-DX2 can treat neuronal diseases (US2019/0298858 A1).

It has been also determined that when AIMP2-DX2 is inserted into an adeno-associated virus and the resultant is introduced into subretinal space, it effectively inhibits choroidal neovascularization.

Disclosed herein are methods of treating retinal degenerative diseases in a subject in need thereof, comprising administering to the subject a pharmaceutically effective amount of a nucleic acid molecule comprising an exon 2-deleted AIMP2 variant (AIMP2-DX2 or DX2). In some embodiments, the nucleic acid molecule is a viral vector or a nonviral vector, e.g., a recombinant vector.

Disclosed herein are methods of treating retinal degenerative diseases in a subject in need thereof, comprising administering to the subject a pharmaceutically effective amount of a recombinant vector comprising an exon 2-deleted AIMP2 variant (AIMP2-DX2 or DX2) gene.

In some embodiments, the retinal degenerative disease is retinitis pigmentosa, Leber's congenital amaurosis, Cone-rod dystrophy, glaucoma, or diabetic retinopathy. In some embodiments, the retinal degenerative disease precedes or is accompanied by Parkinson's disease, Alzheimers's disease, or amyotrophic lateral sclerosis. In some embodiments, the retinal degenerative disease is not age-related macular disease. In some embodiments, the methods of treating retinal degenerative diseases does not include treating an age-related macular disease. In some embodiments, the AMD is wet AMD. In some embodiments, the AMD is dry AMD.

Disclosed herein are methods of decreasing vascular leakage, reducing choroidal neovascularization area, reducing choroidal neovascularization formation, or reducing VEGF expression in the eye or area surrounding the eye in a subject suffering from a retinal degenerative disease, comprising administering to the subject a pharmaceutically effective amount of a recombinant vector comprising an exon 2-deleted AIMP2 variant (AIMP2-DX2) gene.

The recombinant vectors as disclosed herein can further comprise an miR-142 target sequence. The vector can further comprise a promoter operably linked to the AIMP2-DX2. In some embodiments, the promoter is a Retrovirus (LTR) promoter, cytomegalovirus (CMV) promoter, Rous sarcoma virus (RSV) promoter, MT promoter, EF-1 alpha promoter, UB6 promoter, chicken beta-actin promoter, CAG promoter, RPE65 promoter, Synapsin promoter, MeCP2 promoter, CaMKII promoter, Hb9 promoter, or opsin promoter.

In the methods disclosed herein, in some embodiments, the recombinant vectors can comprise exon 2-deleted AIMP2 variant (AIMP2-DX2) gene and an miR-142 target sequence. The miR-142 target sequence can be 3′ to the AIMP2-DX2 gene. The vectors described herein can express AIMP2-DX2 in neuronal cells but not in hematopoietic cells, such as leukocytes and lymphoid cells.

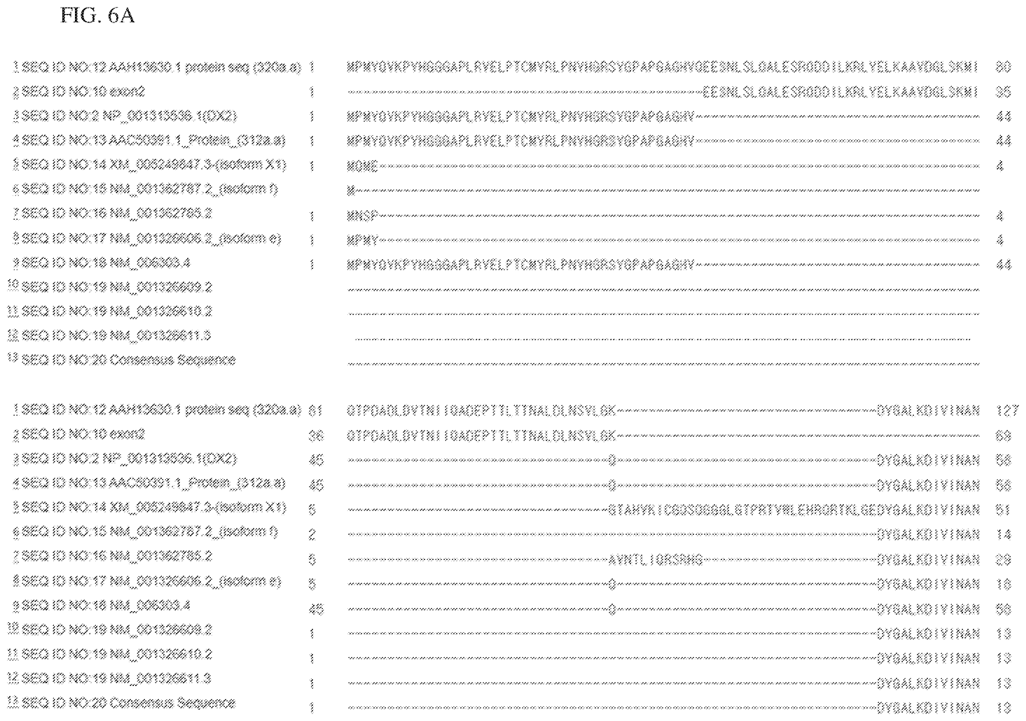

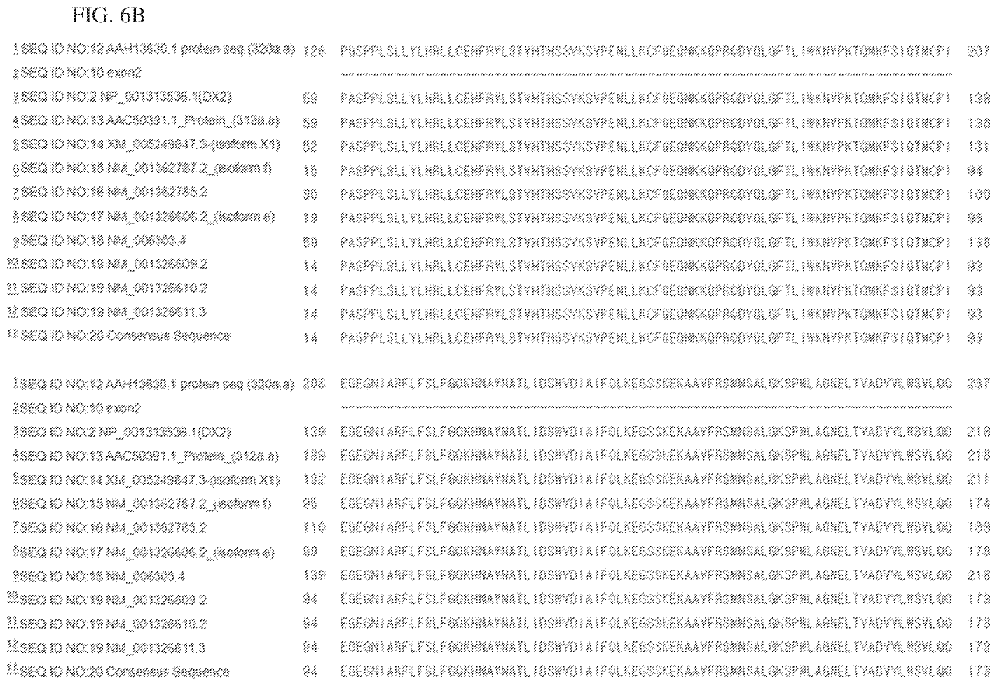

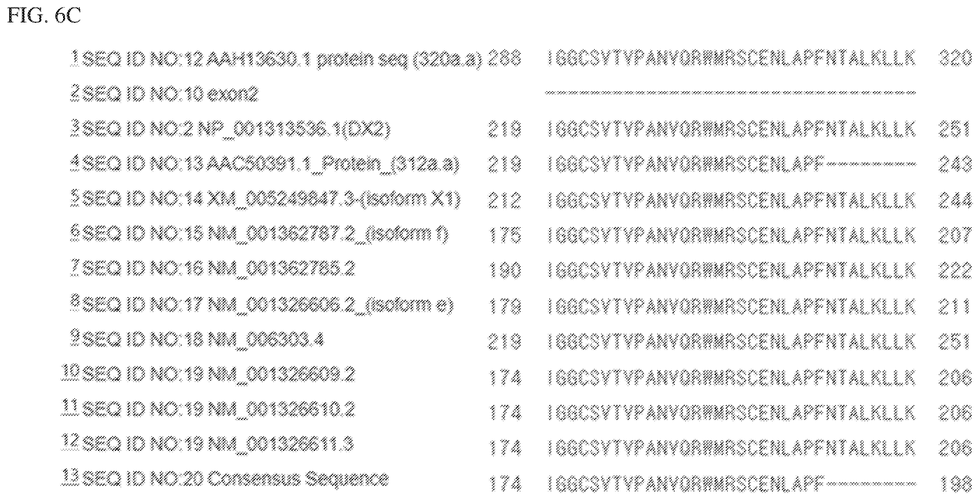

The AIMP2-DX2 polypeptide (SEQ ID NO:2) is a splice variant of AIMP2 (e.g., aa sequence of SEQ ID NO:12; e,g., nt sequence of SEQ ID NO:3), in which the second exon (SEQ ID NO: 10; nt sequence of SEQ ID NO:4) of AIMP2 is omitted. In some embodiments, the AIMP2-DX2 gene has a nucleotide sequence set forth in SEQ ID NO:1, and the AIMP2-DX2 polypeptide has an amino acid sequence set forth in SEQ ID NO:2. Variants or isoforms of the AIMP2-DX2 polypeptide are also known and can be determined by those in the art (see, e.g., SEQ ID NOS: 13-19. For example, FIGS. 6A-6C show a comparison of AIMP2 (SEQ ID NO:2) and variants, SEQ ID NOS: 13-19, as well as a consensus or core sequence of AIMP2 or AIMP2-DX2 (SEQ ID NO: 20).

In some embodiments, the AIMP2-DX2 gene can comprise a nucleotide sequence encoding an amino acid sequence that is at least 90% identical, at least 93% identical, at least 95% identical, at least 96% identical, at least 97% identical, at least 98% identical, at least 99% identical to SEQ ID NO: 2, 13, 14, 15, 16, 17, 18, 19, or 20, or any ranges of % identity therein. The AIMP2-DX2 gene can comprise a nucleotide sequence encoding an amino acid sequence of SEQ ID NO:2, 13, 14, 15, 16, 17, 18, 19, or 20.

The AIMP2-DX2 gene can comprise a nucleotide sequence at least 90% identical, at least 93% identical, at least 95% identical, at least 96% identical, at least 97% identical, at least 98% identical, at least 99% identical to a nucleotide sequence of SEQ ID NO:1, or any ranges of % identity therein. The AIMP2-DX2 gene can comprise a nucleotide sequence of SEQ ID NO:1.

In some embodiments, the AIMP2-DX2 gene does not have an exon comprising a nucleotide sequence encoding an amino acid sequence having at least 70%, at least 75%, at least 80%, at least 85%, at least 90%, at least 95%, at least 96%, at least 97%, at least 98%, at least 99%, or 100% identity to SEQ ID NO:10 or 11. In some embodiments, the AIMP2-DX2 gene does not have an exon comprising a nucleotide sequence encoding an amino acid sequence of SEQ ID NO: 10 or 11. In some embodiments, the AIMP2-DX2 gene does not have an exon comprising a nucleotide sequence having at least 70%, at least 75%, at least 80%, at least 85%, at least 90%, at least 95%, at least 96%, at least 97%, at least 98%, at least 99%, or 100% identity to SEQ ID NO:4.

In some embodiments, the nucleic acid molecule comprising AIMP2-DX2 can optionally comprise an miR-142 target sequence. The miR-142 target sequence (miR-142T) can comprise a nucleotide sequence comprising ACACTA. The miR-142 target sequence can comprise a nucleotide sequence comprising ACACTA and 1-17 additional contiguous nucleotides of SEQ ID NO: 5. For example, the miR-142 target sequence can comprise a nucleotide sequence comprising ACACTA and a sum of 1, 2, 3, 4, 5, 6, 7, 8, 9, 10, 11, 12, 13, 14, 15, 16, or 17 additional nucleotides that are contiguous 5′ or 3′ of ACACTA as shown in SEQ ID NO:5.

The miR-142 target sequence can comprise a nucleotide sequence at least 50% identical, at least 60% identical, at least 70% identical, at least 80% identical, at least 90% identical, at least 90% identical, at least 93% identical, at least 95% identical, at least 96% identical, at least 97% identical, at least 98% identical, at least 99% identical, or 100% identical to a nucleotide sequence of SEQ ID NO:5 (TCCATAAAGTAGGAAACACTACA; miR-142-3 pT). The miR-142 target sequence can comprise a nucleotide sequence of SEQ ID NO:5.

The miR-142 target sequence can comprise a nucleotide sequence comprising ACTTTA. The miR-142 target sequence can comprise a nucleotide sequence comprising ACTTTA and 1-15 additional contiguous nucleotides of SEQ ID NO:7. For example, the miR-142 target sequence can comprise a nucleotide sequence comprising ACTTTA and a sum of 1, 2, 3, 4, 5, 6, 7, 8, 9, 10, 11, 12, 13, 14, or 15 additional nucleotides that are contiguous 5′ or 3′ of ACTTTA as shown in SEQ ID NO:7.

The miR-142 target sequence can comprise a nucleotide sequence at least 50% identical, at least 60% identical, at least 70% identical, at least 80% identical, at least 90% identical, at least 90% identical, at least 93% identical, at least 95% identical, at least 96% identical, at least 97% identical, at least 98% identical, at least 99% identical, or 100% identical to a nucleotide sequence of SEQ ID NO:7 (AGTAGTGCTTTCTACTTTATG; miR-142-5 pT). The miR-142 target sequence can comprise a nucleotide sequence of SEQ ID NO:7.

An example miR142-3 pT mutant sequence is:

(SEQ ID NO: 25)
ccgctgcagtgtgacagtgccagccaatgtgcagaggtggatgaggtct
tgtgaaaacctggctccttttaacacggccctcaagctccttaagtgac
cagaagcttgctagctccataaagtaggaCCACTGCAatcactccataa
agtaggaCCACTGCAagatatctccataaagtaggaCCACTGCAatcac
tccataaagtaggaCCACTGCAaaagcttgtagggatccgcc.

A mutant sequence refers to one or more regions, e.g., four regions of core sequences of miR142 3 pT that are substituted as follows: (5′-AACACTAC-3′→5′-CCACTGCA-3′). Inhibition of DX2 expression in vector transfected HEK293 cells was observed with the miR142-3p×1 repeat (100 μmol) miR142-3p target sequence and as the number of core binding sequence in miR142-3p target seq are increased, miR142-3p inhibition on DX2 expression was also increased. The Tseq x3 core sequence containing vector showed significant inhibition, whereas no inhibition was observed for the mutated 3× sequence.

A microRNA (miRNA) is a non-coding RNA molecule that functions to control gene expression. MiRNAs function via base-pairing with complementary sequences within mRNA molecules, i.e., a miRNA target sequences. miRNAs can bind to target messenger RNA (mRNA) transcripts of protein-coding genes and negatively control their translation or cause mRNA degradation. At present, more than 2000 human miRNAs have been identified and miRbase databases are publicly available. Many miRNAs are expressed in a tissue-specific manner and have an important roles in maintaining tissue-specific functions and differentiation.

MiRNA acts at the post-transcription stage of the gene and, in the case of mammals, and it is known that approximately 60% of the gene expression is controlled by miRNA. miRNA plays an important role in a diverse range of processes within a living body and has been disclosed to have correlation with cancer, cardiac disorders and nerve related disorders. For example, miR-142-3p and miR-142-5p exist in miR-142 and any of the target sequences thereof can be used. Thus, “miR-142” or “miRNA-142” refers to, e.g., miR-142-3p and/or miR-142-5p, and can bind to the miR-142 target sequence, e.g., miR-142-3 pT or miR-142-5 pT.

The miR-142 target sequence can be 5′ or 3′ to the AIMP2-DX2 gene.

For example, “miR-142-3p” can exist in the area at which translocation of its gene occurs in aggressive B cell leukemia and is known to express in hemopoietic tissues (bone marrow, spleen and thymus, etc.). In addition, miR-142-3p is known to be involved in the differentiation of hemopoietic system with confirmation of expression in the liver of fetal mouse (hemopoietic tissue of mouse).

In some embodiments, the miR-142-3p and/or miR-142-5p target sequence is repeated at least 2-10 times, at least 2-8 times, at least 2-6 times, at least 4 times, or any range or number of times thereof.

As an example, the miR-142-3p, e.g., having a nucleotide sequence of SEQ ID NO:23, can have a corresponding target sequence, e.g., a miR-142-3p target sequence (miR-142-3 pT) having a nucleotide sequence of SEQ ID NO:5 but not limited thereto. The miR-142-5p, e.g., having a nucleotide sequence of SEQ ID NO:24 can have a corresponding target sequence, e.g., a miR-142-5p target sequence (miR-142-5 pT) having a nucleotide sequence of SEQ ID NO:7 but not limited thereto.

In some embodiments, an miR-142-3p can have a nucleotide sequence of SEQ ID NO:23 and an miR-142-5p can have a nucleotide sequence of SEQ ID NO:24.

Disclosed herein are recombinant vectors that can control the side effect of over-expression of the AIMP2-DX2 variant by inserting an miR-142-3p target sequence and/or miR-142-5p target sequence (miR-142-3 pT and/or miR-142-5 pT, respectively) into a terminal end of AIMP2-DX2 and controlling suppression of AIMP2-DX2 expression in CD45-derived cells, in particular, the lymphatic system and leukocytes. Thus, the expression of AIMP2-DX2 variant can be restricted to only in the injected neuronal cells and tissues but not in non-neuronal hematopoietic cells, the major population in the injected tissue areas. MiR142-3p is expressed only in hematopoietic cells.

Disclosed herein are recombinant vectors containing a target sequence for miR-142-3p and/or miR-142-5p. Disclosed herein are recombinant vectors comprising an exon 2-deleted AIMP2 variant (AIMP2-DX2) gene and miR-142-3p and/or miR-142-5p target sequences as disclosed herein.

The term “recombinant vector” refers to a vector that can encode a target protein or RNA in appropriate host cells, or a gene construct that contains essential operably linked control factor(s) to enable the inserted gene to be expressed appropriately.

The term “operably linked” refers to functional linkage between the nucleic acid expression control sequence and nucleic acid sequence that codes the targeted protein and RNA to execute general functions. For example, it can affect the expression of nucleic acid sequence that codes promoter and protein or RNA that has been linked for operability of the nucleic acid sequence. Operable linkage with recombinant vector can be manufactured by using gene recombinant technology, which is known well in the corresponding technology area, and uses generally known enzymes in the corresponding technology area for the area-specific DNA cutting and linkage.

The recombinant vectors can further comprise a promoter operably linked to a AIMP2-DX2 as disclosed herein. In some embodiments, the promoter is a Retrovirus (LTR) promoter, cytomegalovirus (CMV) promoter, Rous sarcoma virus (RSV) promoter, MT promoter, EF-1 alpha promoter, UB6 promoter, chicken beta-actin promoter, CAG promoter, RPE65 promoter, Synapsin promoter, MeCP2 promoter, CaMKII promoter, Hb9 promoter, or opsin promoter.

The recombinant vector can additionally contain heterogeneous promoter and operably linked heterogeneous gene in the promoter.

“Heterogeneous gene” as used herein can include protein or polypeptide with biologically appropriate activation, and encrypted sequence of the targeted product such as immunogen or antigenic protein or polypeptide, or treatment activation protein or polypeptide.

Polypeptides can supplement deficiency or absent expression of endogenous protein in host cells. The gene sequence can be induced from a diverse range of suppliers including DNA, cDNA, synthesized DNA, RNA or its combinations. The gene sequence can include genome DNA that contains or does not contain a natural intron. In addition, the genome DNA can be acquired along with promoter sequence or polyadenylated sequence. Genome DNA or cDNA can be acquired in various methods. Genome DNA can be extracted and purified from appropriate cells through methods publicly notified in the corresponding area. Alternatively, mRNA can be used to produce cDNA by reverse transcription or other methods by being separated from the cells. Alternatively, polynucleotide sequence can contain sequence that is complementary to RNA sequence, e.g., antisense RNA sequence, and the antisense RNA can be administered to individual to suppress expression of complementary polynucleotide in the cells of individuals.

For example, the heterogeneous gene is an AIMP-2 splicing variant with the loss of exon 2 and miR-142-3p target sequence can be linked to 3′ UTR of the heterogeneous gene. The sequence of the AIMP2 protein (312aa version: AAC50391.1 or GI: 1215669; 320aa version: AAH13630.1, GI: 15489023, BC0 13630.1) are described in the literature (312aa version: Nicolaides, N.C., Kinzler, K. W. and Vogelstein, B. Analysis of the 5′ region of PMS2 reveals heterogeneous transcripts and a novel overlapping gene, Genomics 29 (2), 329-334 (1995)/320 aa version: Generation and initial analysis of more than 15, 000 full-length human and mouse cDNA sequences, Proc. Natl. Acad. Sci. U.S.A. 99 (26), 16899-16903 (2002)).

The term “AIMP2 splicing variant” refers to the variant generated due to partial or total loss of exon 2 among exons 1 to 4. As such, the variant signifies interference of the normal function of AIMP2 by forming AIMP2 protein and heterodimer. The injected AIMP2-DX2 gene is rarely expressed in tissues other than the injected tissue. However, as an additional safety measure, an miR142 target sequence can be inserted to completely block the possibility of AIMP2-DX2 being expressed in hematopoietic cells, the major population of non-neuronal cells in the injected tissue area.

The recombinant vector can include SEQ ID NOS: 1 and 5.

The term “% of sequence homology,” “% identity,” or “% identical” to a nucleotide or amino acid sequence can be, e.g., confirmed by comparing the 2 optimally arranged sequence with the comparison domain and some of the nucleotide sequences in the comparison domain can include addition or deletion (that is, gap) in comparison to the reference sequence on the optimal arrange of the 2 sequences (does not include addition or deletion).

Proteins as disclosed herein not only include those with its natural type amino acid sequence but also those with variant amino acid sequences.

Variants of the protein signifies proteins with difference sequences due to the deletion, insertion, non-conservative or conservative substitution or their combinations of the natural amino acid sequence and more than 1 amino acid residue. Amino acid exchange in protein and peptide that does not modify the activation of the molecule in overall is notified in the corresponding area (H. Neurath, R. L. Hill, The Proteins, Academic Press, New York, 1979).

The protein or its variant can be manufactured through natural extraction, synthesis (Merrifield, J. Amer. Chem. Soc. 85:2149-2156, 1963), or genetic recombination on the basis of the DNA sequence (Sambrook et al, Molecular Cloning, Cold Spring Harbour Laboratory Press, New York, USA, 2^{nd} Ed., 1989).

Amino acid mutations can occur on the basis of the relative similarity of the amino acid side chain substituent such as hydrophilicity, hydrophobicity, electric charge and size, etc. In accordance with the analysis of the size, shape and types of amino acid side chain substituent, it can be discerned that arginine, lysine and histidine are residues with positive charge; alanine, glycine and serine have similar sizes; phenylalanine, tryptophan and tyrosine have similar shapes. Therefore, on the basis of such considerations, arginine, lysine and histidine; alanine, glycine and serine; and phenylalanine, tryptophan and tyrosine can be deemed functional equivalents biologically.

In introducing one or more mutations, hydrophobic index of amino acid can be considered. Hydrophobic index is assigned to each amino acid according to hydrophobicity and charge: isoleucine (+4.5); valine (+4.2); leucine (+3.8); phenylalanine (+2.8); cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5)

In assigning interactive biological function of protein, hydrophobic amino acid index is very important. It is possible to have similar biological activation only if a substitution is made with an amino acid with a similar hydrophobic index. In the event of introducing a mutation by making reference to the hydrophobic index, substitution between amino acids with hydrophobic index differences within ±2, within ±1, or within ±0.5.

Meanwhile, it is also well known that substitution between amino acids with similar hydrophilicity value can induce proteins with equivalent biological activation. As indicated in U.S. Pat. No. 4,554,101, the following hydrophilic values are assigned to each of the amino acid residues: arginine (±3.0); lysine (±3.0); aspartate (±3.0±1); glutamate (±3.0±1); serine (±0.3); asparagine (±0.2); glutamine (±0.2); glycine (0); threonine (−0.4); proline (−0.5+1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4).

In the event of introducing one or more mutations by making reference to hydrophilic values, substitutions can be made between amino acids with hydrophilic value differences within ±2, within ±1, or within ±0.5. but not limited thereto.

Amino acid exchange in protein that does not modify the activation of molecule in overall is notified in the corresponding area (H. Neurath, R. L. Hill, The Proteins, Academic Press, New York, 1979). The most generally occurring exchanges are those between the amino acid residues including Ala/Ser, Val/Ile, Asp/Glu, Thr/Ser, Ala/Gly, Ala/Thr, Ser/Asn, Ala/Val, Ser/Gly, Thy/Phe, Ala/Pro, Lys/Arg, Asp/Asn, Leu/Ile, Leu/Val, Ala/Glu and Asp/Gly. Vector system can be constructed through diverse methods announced in the corresponding industry. The specific methods are described in Sambrook et al. (2001), Molecular Cloning, A Laboratory Manual, Cold Spring Harbor Laboratory Press.

Vectors disclosed herein can be constructed as a typical vector for cloning or for expression. In addition, the vectors can be constructed with prokaryotic or eukaryotic cells as the host. If the vector is an expression vector and prokaryotic cell is used as the host, it is general to include powerful promoter for execution of transcription (e.g., tac promoter, lac promoter, lacUV5 promoter, 1pp promoter, pL X promoter, pRX promoter, rac5 promoter, amp promoter, recA promoter, SP6 promoter, trp promoter and T7 promoter, etc.), ribosome binding site for commencement of decoding and transcription/decoding termination sequence. In the case of using E. coli (e.g., HB101, BL21, DH5a, etc.) as the host cell, promoter and operator site of the tryptophan biosynthesis route of E. coli (Yanofsky, C. (1984), J. Bacteriol., 158:1018-1024) and left directional promoter of phage X (pLX promoter, Herskowitz, I. and Hagen, D. (1980), Ann. Rev. Genet., 14:399-445) can be used as the control site.

Meanwhile, vectors that can be used can be more than 1 type, such as a virus vector, linear DNA, or plasmid DNA.

“Virus vector” refers to a virus vector capable of delivering gene or genetic substance to the desired cells, tissue and/or organ.

Although the virus vectors can include more than 1 species from the group composed of Adenovirus, Adeno-associated virus, Lentivirus, Retrovirus, HIV (Human immunodeficiency virus), MLV (Murine leukemia virus), ASLV (Avian sarcoma/leukosis), SNV (Spleen necrosis virus), RSV (Rous sarcoma virus), MMTV (Mouse mammary tumor virus) and Herpes simplex virus, it is not limited thereto. In some embodiments, the viral vector can be an adeno-associated virus (AAV), adenovirus, lentivirus, retrovirus, vaccinia virus, or herpes simplex virus vector.

Although Retrovirus has an integration function for the genome of host cells and is harmless to the human body, it can have characteristic including suppressing functions of normal cells at the time of integration, ability to infect a diverse range of cells, ease of proliferation, accommodate approximately 1-7 kb of external gene, and generate duplication deficient virus. However, Retroviruses can also have disadvantages including difficulties in infecting cells after mitotic division, gene delivery under an in vivo condition, and need to proliferate somatic cells under in vitro condition. In addition, Retroviruses have the risk of spontaneous mutations as it can be integrated into proto-oncogene, thereby presenting the possibility of cell necrosis.

Meanwhile, Adenoviruses have various advantages as a cloning vector including duplication even in nucleus of cells in medium level size, clinically nontoxic, stable even if external gene is inserted, no rearrangement or loss of genes, transformation of eukaryotic organism and stably undergoes expression at high level even when integrated into host cell chromosome. Good host cells of Adenoviruses are the cells that are the causes of hemopoietic, lymphatic and myeloma in humans. However, proliferation is difficult since it is a linear DNA and it is not easy to recover the infected virus along with low infection rate of virus. In addition, expression of the delivered gene is most extensive during 1-2 weeks with expression sustained over the 3-4 weeks only in some of the cells. Another issue is that it has high immuno-antigenicity.

Adeno-associated virus (AAV) has been preferred in recent years since it can supplement the aforementioned problems and has a lot of advantages as gene therapy agent. It is also referred to as adeno-satellite virus. The diameter of adeno-associated virus particle is 20 nm and is known to have almost no harm to the human body. As such, its sales as gene therapy agent in Europe were approved.

AAV is a provirus with single strand that needs auxiliary virus for duplication and AAV genome has 4,680 bp that can be inserted into specific area of the chromosome 19 of the infected cells. Trans-gene is inserted into the plasma DNA connected by the 2 inverted terminal repeat (ITR) sequence section with 145 bp each and signal sequence section. Transfection is executed along with other plasmid DNA that expresses the AAV rep and cap sections, and Adenovirus is added as an auxiliary virus. AAV has the advantages of a wide range of host cells that deliver genes, little immunological side effects at the time of repetitive administration and long gene expression period. Moreover, it is safe even if the AAV genome is integrated with the chromosome of host cells and does not modify or rearrange the gene expression of the host.

The Adeno-associated virus is known to have a total of 4 serotypes. Among the serotypes of many Adeno-associated viruses that can be used in the delivery of the target gene, the most widely researched vector is the Adeno-associated virus serotype 2 and is currently used in the delivery of clinical genes of cystic fibrosis, hemophilia and Canavan's disease. In addition, recently, the potential of recombinant adeno-associated virus (rAAV) is increasing in the area of cancer gene therapy (Du 2013). In some embodiments, the Adeno-associated virus serotype 2 can be used. Although it is possible to select and apply appropriate viral vector, it is not limited to this.

In addition, if the vectors are expression vectors and use eukaryotic cells as the host, promoter derived from the genome of mammalian cells (e.g., metallothionein promoter) or promoter derived from mammalian virus (e.g., post-adenovirus promoter, vaccine virus 7.5K promoter, SV40 promoter, cytomegalovirus promoter and HSV TK promoter) can be used. Specifically, although it can include more than 1 species selected from the group composed of promoters selected from the group composed of LTR of Retrovirus, cytomegalovirus (CMV) promoter, Rous sarcoma virus (RSV) promoter, MT promoter, EF-1 alpha promoter, UB6 promoter, chicken beta-actin promoter, CAG promoter, RPE65 promoter and opsin promoter, it is not limited to these. Moreover, it generally has polyadenylated sequence as the transcription termination sequence.

Vectors disclosed herein can be fused with other sequences as needed to make the purification of the protein easier. Although a fused sequence such as glutathione S-transferase (Pharmacia, USA), maltose binding protein (NEB, USA), FLAG (IBI, USA) and 6×His (hexahistidine; Qiagen, USA), etc. can be used, e.g., it is not limited to these. In addition, expression vectors can include tolerance gene against antibiotics generally used in the corresponding industry as the selective marker including Ampicillin, Gentamycin, Carbenicillin, Chloramphenicol, Streptomycin, Kanamycin, Geneticin, Neomycin and Tetracycline, as examples.

In addition, disclosed herein are gene carriers including the recombinant vector containing a target sequence (miR-142-3 pT and/or miR-142-5 pT) for miR-142, such as miR-142-3p and/or miR-142-5p, respectively.

The term “gene transfer” includes delivery of genetic substances to cells for transcription and expression in general. Its method is ideal for protein expression and treatment purposes. A diverse range of delivery methods such as DNA transfection and virus transduction are announced. It signifies virus-mediated gene transfer due to the possibility of targeting specific receptor and/or cell types through high delivery efficiency and high level of expression of delivered genes, and, if necessary, nature-friendliness or pseudo-typing.

The gene carriers can be transformed entity that has been transformed into the recombinant vector, and transformation includes all methods of introducing nucleic acid to organic entity, cells, tissues or organs and as announced in the corresponding area, it is possible to select and execute appropriate standard technology in accordance with the host cells. Although such methods include electroporation, fusion of protoplasm, calcium phosphate (CaP0₄) sedimentation, calcium chloride (CaCl₂)) sedimentation, mixing with the use of silicone carbide fiber, agribacteria-mediated transformation, PEG, dextran sulphate and lipofectamin, etc., it is not limited to these.

The gene carriers are for the purpose of expression of heterogeneous genes in neuron. As such it suppresses the expression of the heterogeneous gene in CD45-derived cells and can increase the expression of heterogeneous gene in brain tissue. The majority of the CD45 are transmembrane protein tyrosine phosphatase situated at the hematopoietic cell. Cells can be defined in accordance with the molecules situated on the cell surface and CD45 is the cell marker for all leukocyte groups and B lymphocytes. The gene carrier is not expressed in the CD45-derived cells, in particular, in lymphoid and leukocyte range of cells.

The gene carriers can additionally include carrier, excipient or diluent allowed to be used pharmacologically.

In addition, disclosed herein are methods of delivering and expressing the heterogeneous gene in the neuron that includes the stage of introducing the recombinant vector into the corresponding entity.

The methods of delivery disclosed herein can increase the expression of heterogeneous gene in cerebral tissues and control heterogeneous gene expression in other tissues.

In addition, disclosed herein are vectors comprising 1) a promoter; 2) a nucleotide sequence that encodes a target protein linked with the promoter to enable operation; and 3) an expression cassette that includes the nucleotide sequence targeting miR-142-3p inserted into 3′UTR of the nucleotide sequence. In some embodiments, the vectors can comprise 1) a promoter; 2) a nucleotide sequence that encodes a target protein linked with the promoter to enable operation; and 3) an expression cassette that includes the nucleotide sequence targeting miR-142-5p inserted into 3′UTR of the nucleotide sequence.

The term “expression cassette” refers to the unit cassette that can execute expression for the production and secretion of the target protein operably linked with the downstream of signal peptide as it includes gene that encodes the target protein and nucleotide sequence that encodes the promoter and signal peptide. Secretion expression cassette can be used mixed with the secretion system. A diverse range of factors that can assist the efficient production of the target protein can be included in and out of such expression cassette.

In addition, disclosed herein are preventive or therapeutic preparations for retinal degenerative disease that includes a nucleotide sequence that encodes AIMP-2 splicing variant with loss of exon 2 and nucleotide sequence that targets miR-142-3p linked to 3′UTR of the nucleotide sequence.

Accordingly, also disclosed herein are methods of treating retinal degenerative disease in a subject in need thereof, comprising administering any of the vectors disclosed herein. In some embodiments, the retinal degenerative disease does not include AMD. AMD can be is wet AMD. In some embodiments, the AMD is dry AMD.

The vectors disclosed herein can affect, but not limited to, apoptosis inhibition, dyskinesia amelioration, and/or oxidative stress inhibition, and thus prevent or treat retinal degenerative disease.

The term “treatment” includes not only complete treatment of retinal degenerative disease but also partial treatment, improvement and/or reduction in the overall symptoms of AMD as results of application of the pharmacological agent disclosed herein.

The term “prevention” signifies prevention of the occurrence of overall symptoms of retinal degenerative disease in advance by suppressing or blocking the symptoms or phenomenon such as cognition disorder, behavior disorder and destruction of brain nerves by applying pharmacological agents disclosed herein to the entity with degenerative cerebral disorders.

Adjuvants other than the active ingredients can be included additionally to the pharmacological agents disclosed herein. Although any adjuvant can be used without restrictions as long as it is known in the corresponding technical area, it is possible to increase immunity by further including complete and incomplete adjuvant of Freund, for example.

Pharmacological agents disclosed herein can be manufactured in the format of having mixed the active ingredients with the pharmacologically allowed carrier. Here, pharmacologically allowed carrier includes carrier, excipient and diluent generally used in the area of pharmacology. Pharmacologically allowed carrier that can be used for the pharmacological agents disclosed herein include lactose, dextrose, sucrose, sorbitol, mannitol, xylitol, erythritol, malitol, starch, acacia rubber, alginate, gelatin, calcium phosphate, calcium silicate, cellulose, methyl cellulose, polyvinyl pyrrolidone, water, methylhydroxy benzoate, propylhydroxy benzoate, talc, magnesium stearate and mineral oil, but not limited to these.

Pharmacological agents disclosed herein can be used by being manufactured in various formats including oral administration types such as powder, granule, pill, capsule, suspended solution, emulsion, syrup and aerosol, etc., and external application, suppository drug or disinfection injection solution, etc. in accordance with their respective general manufacturing methods.

When manufactured into preparations, diluents or excipients such as filler, extender, binding agent, humectant, disintegrating agent and surfactant, etc., which are used generally, can be used in the manufacturing. Solid preparations for oral administration include pill, tablet, powder, granule and capsule preparations, and such solid preparations can be manufactured by mixing more than 1 excipient such as starch, calcium carbonate, sucrose, lactose and gelatin with the active ingredients. In addition, lubricants such as magnesium stearate and talc can also be used in addition to simple excipients. Liquid preparations for oral administration include suspended solution, solution for internal use, oil and syrup, etc. with the inclusion of various excipients such as humectant, sweetening agent, flavoring and preservative, etc. other than water and liquid paraffin, which are the generally used diluents. Preparations for non-oral administration include sterilized aqueous solution, non-aqueous solvent, suspension agent, oil, freeze dried agent and suppository. Vegetable oil such as propylene glycol, polyethylene glycol and olive oil, and injectable esters such as ethylate can be used as non-aqueous solvent and suspension solution. Agents for suppository can include witepsol, tween 61, cacao oil, laurine oil and glycerogelatin, etc.

Pharmacological agents disclosed herein can be administered into entity through diversified channels. All formats of administration such as oral administration, and intravenous, muscle, subcutaneous and intraperitoneal injection can be used.

In some embodiments, the recombinant vector is administered topically to, by intravitreal injection to, by subconjunctival injection to, or into a subretinal space of the subject.

The methods disclosed herein can further comprise administering to the subject an additional therapeutic agent(s). In some embodiments, the additional therapeutic agent is ranibizumab, aflibercept, and/or bevacizumab.

Desirable doses of administration of therapeutic agents disclosed herein differs depending on various factors including preparation production method, administration format, age, weight and gender of the patient, extent of the symptoms of the disease, food, administration period, administration route, discharge speed and reaction sensitivity, etc. Nonetheless, it can be selected appropriately by the corresponding manufacturer.

Further, for the treatment effects, the skilled medical doctor can determine and prescribe effective dose for the targeted treatment. For example, the treatment routes can include but are not limited to subretinal, intravitreal, intravenous, subcutaneous and muscle injection, and direction injection into the eye, cerebral ventricle or spinal cord by using micro-needle. In some embodiments, the dose per eye can be 1×10⁸ vg (viral genome) to 1×10¹¹ vg, 1×10⁸ vg to 1×10¹⁰ vg, 1×10⁸ vg to 1×10⁹ vg, and any specific doses or ranges of doses therein, e.g., 2×10⁸ vg to 5×10⁸ vg, 2×10⁹ vg to 4×10¹⁰ vg, 4×10⁸ vg to 6×10¹⁰ vg, 5×10⁸ vg to 4×10¹⁰ vg, 5×10⁸ vg to 1×10¹¹ vg, 1×10⁹ vg to 5×10¹⁰ vg, 2×10⁹ vg to 1×10¹⁰ vg, 5×10⁹ vg to 1×10¹⁰ vg, or 5×10⁹ vg to 8×10¹⁰ vg. Multiple injections and repetitive administrations are possible per day, e.g., the effective dose can be, e.g., per eye, 0.05 to 15 mg/kg in the case of vector, 5×10¹¹ to 3.3×10¹⁴ viral particle (2.5×10¹² to 1.5×10¹⁶ IU)/kg in the case of recombinant virus and 5×10² to 5×10⁷cells/kg in the cells. Desirably, the doses are 0.1 to 10 mg/kg in the case of vector, 5×10¹² to 3.3×10¹³ particles (2.5×10¹³ to 1.5×10¹⁵ IU)/kg in the case of recombinant virus and 5×10³ to 5×10⁶ cells/kg in the case of cells at the rate of 2 to 3 administrations per week. The dose is not strictly restricted. Rather, it can be modified in accordance with the condition of the patient and the extent of manifestation of the neural disorders. Effective dose for other subcutaneous fat and muscle injection, and direct administration into the affected area is 9×10¹⁰ to 3.3×10¹⁴ recombinant viral particles with the interval of 10 cm and at the rate of 2-3 times per week. The dose is not strictly restricted. Rather, it can be modified in accordance with the condition of the patient and the extent of manifestation of the neural disorders. More specifically, pharmacological agents disclosed herein can include 1×10¹⁰ to 1×10¹² vg (virus genome)/mL of recombinant adeno-associated virus and, generally, it is advisable to inject 1×10¹² vg once every 2 days over 2 weeks. It can be administered once a day or by dividing the dose for several administrations throughout the day. In some embodiments, the vectors can be administered in a dose of 0.1×10⁸ vg to 500×10⁸ vg, 1×10⁸ vg to 100×10⁸ vg, 1×10⁸ vg to 10×10⁸ vg, e.g., 5×10⁸ vg, or any specific amounts or ranges derived therefrom. For IV injections, e.g., vg can be translated to doses for humans based on body weight for IV injection. For local tissue injections, e.g., vg can also be translated to doses for humans based on the target cell number and effective MOI (multiplicity of infection).

In some embodiments, the vectors disclosed herein can be injected to a subject by, e.g., subretinal injection, intravitreal injection, or subchoroidal injection. The injection can be in the form of a liquid. In other embodiments, the vectors disclosed herein can be administered to a subject in the form of eye drops or ointment.

The nucleic acid molecules comprising AIMP2-DX2, such as vectors, can be delivered by methods well known in the art. The nucleic acid molecules comprising AIMP2-DX2 can also be optionally combined with other gene or nucleic acids delivery tools or materials. Delivery methods can include but are not limited to: viral vectors and non-viral vectors; chemical conjugation with Galnac, cell-penetrating peptides, nucleic acids or glycopeptide mediated tools, or synthetic compound vectors; lipid-mediated delivery such as lipid nanoparticle encapsulation; inorganic vector mediated delivery such as inorganic vectors; biological delivery such as exosome mediated delivery; physical delivery such as by micro/nano needles, pressure-perfusion, microprojectiles, electrical energy/electroporation/iontophoresis, sonoporation, magnetoporation, or optoporation/photodynamic energy.

The pharmacological preparations can be produced in a diverse range of orally and non-orally administrable formats. In some embodiments, the vector disclosed herein can be administered to the brain or spinal cord. In some embodiments, the vectors disclosed herein can be administered to the brain by stereotaxic injection.

Orally administrative agents include pills, tablets, hard and soft capsules, liquid, suspended solution, oils, syrup and granules, etc. These agents can include diluent (e.g., lactose, dextrose, sucrose, mannitol, sorbitol, cellulose and/or glycine) and glidents (e.g., silica, talc, and stearic acid and its magnesium or calcium salts, and/or polyethylene glycol) in addition to the active ingredients. In addition, the pills can contain binding agents such as magnesium aluminum silicate, starch paste, gelatin, tragacanthin, methyl cellulose, sodium carboxymethyl cellulose and/or polyvinyl pyrrolidine, and, depending on the situation, can contain disintegration agent such as starch, agar, alginic acid or its sodium salt or similar mixture and/or absorbent, coloring, flavor and sweetener. The agents can be manufactured by general mixing, granulation or coating methods.

In addition, injection agents are the representative form of non-orally administered preparations. Solvents for such injection agents include water, Ringer's solution, isotonic physiological saline and suspension. Sterilized fixation oil of the injection agent can be used as solvent or suspension medium, and any non-irritating fixation oil including mono- and di-glyceride can be used for such purpose. In addition, the injection agent can use fatty acids such as oleic acid.

The invention will be explained in more detail by using the following execution examples below. However, the following execution examples are only for the purpose of specifying the contents of the invention and do not limit the application of the invention to such examples.

### EXAMPLES

### Example 1. Production of the Recombinant Vector

The majority of CD45 are transmembrane protein tyrosine phosphatase of the hematopoietic cell, which can be used to define the cells in accordance with the molecule on the cell surface. CD45 is a marker for all leukocyte groups and B lymphocytes. A recombinant vector has been produced that is expressed specifically and only in neurons without being expressed in CD45-derived cells, in particular, lymphoid and leukocyte cells. The recombinant vector contains a splicing variant in which exon 2 of the Aminoacyl tRNA Synthetase Complex Interacting Multifunctional Protein 2 (AIMP2) has been deleted and an miRNA capable of controlling the expression of the AIMP2 splicing variant.

The recombinant vector was produced as a distribution safety measure in order to induce specific expression of the AIMP2 splicing variant in injected neuronal tissues. Also this was done to completely block any possibility of AIMP2-DX2 being expressed in hematopoietic cells, which is the major population of non-neuronal cells in the injected tissue area.

### Example 1-1. Production of AIMP2 Variant

AIMP2 is one of the proteins involved in the formation of aminoacyl-tRNA synthetase (ARSs) and acts as a multifactorial apoptotic protein. In order to construct a plasmid that expresses the variant in which exon 2 of the AIMP2 has been deleted, cDNA of AIMP2 splicing variant was cloned into pcDNA3.1-myc. The sub-cloning in pcDNA3.1-myc was executed by using EcoR1 and Xho1 after having amplified the AIMP2 splicing variant by using a primer having EcoR1 and Xho1 linker attached to the H322 cDNA.

AIMP2 variant having a nucleotide sequence of SEQ ID NO:1 and an amino acid sequence of SEQ ID NO:2 was used.

### Example 1-2. Sorting of miRNA and Selection of its Target Sequence

As mentioned above, as a distribution safety measure, the recombinant vector was produced as above in order to confine the expression of the AIMP2 variant in injected neuronal cells and to completely block the possibility of AIMP2-DX2 being expressed in hematopoietic cells, the major population of non-neuronal cells in the injected tissue area.

For this purpose, miR-142-3p that is specifically expressed only in hematopoietic cells that generate leukocyte and lymphoid related cells was selected as the target. In order to produce the sequence that targets only the miR-142-3p, microarray data of mouse B cells and computer programming of genes targeted by miR 142-3p (mirSVR score) were used. The miR-142-3p is a nucleotide sequence indicated SEQ ID NO. The miR-142-3p target sequence of SEQ ID NO:5 binds to miR-142-3p.

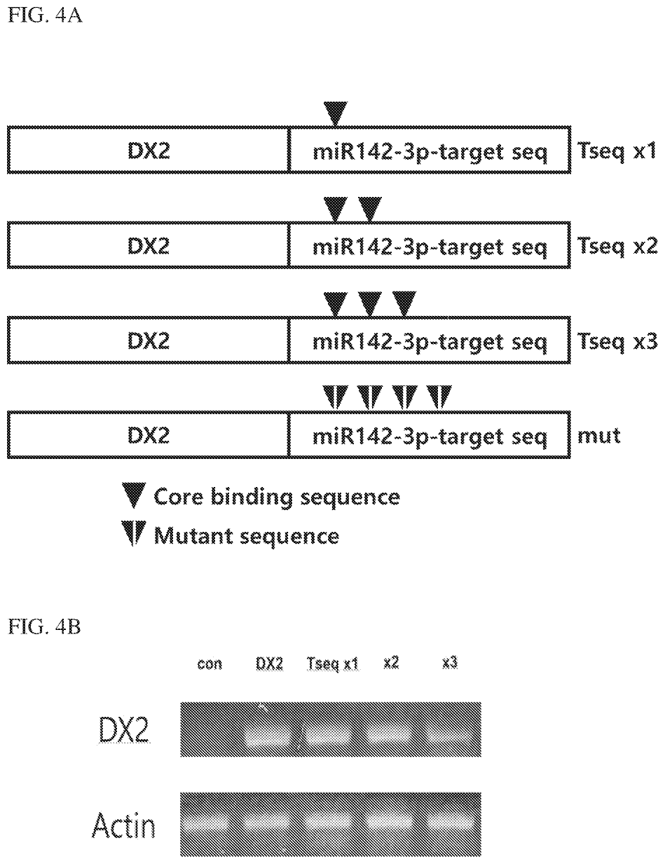

The miR-142-3p target sequence includes Nhe 1 and Hind III, Bmt 1 site sequence (ccagaagcttgctagc; SEQ ID NO:21) and Hind H site sequence (aagcttgtag; SEQ ID NO:22). The miR-142-3 pT can comprise the nucleotide sequence of SEQ ID NO:5 that has been repeated 4 times with the linkers (tcac and gatatc) that connects them (FIG. 4; SEQ ID NO:6).

### Example 1-3. Production of the Recombinant Vector

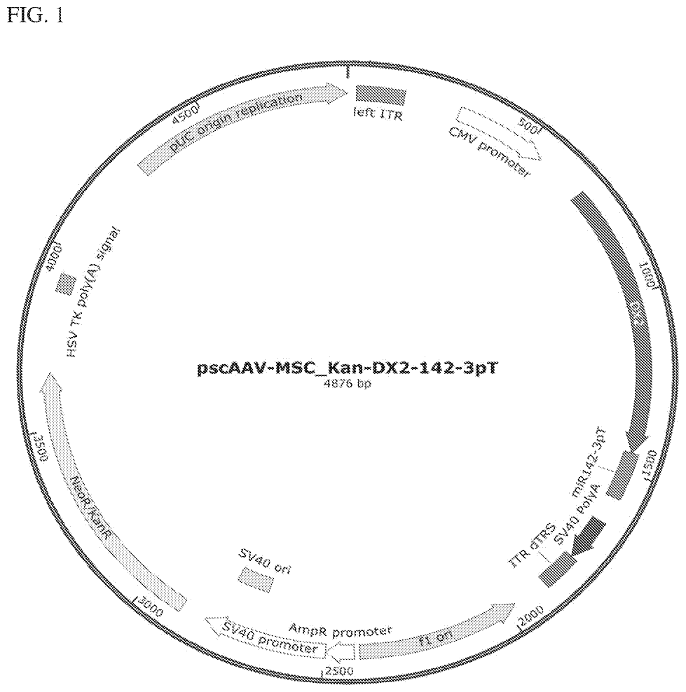

In order to produce the recombinant vector, the miR-142-3p target sequence (SEQ ID NO:5) was inserted into 3′UTR of the AIMP2 variant (sequence number of 1). Connecting of the AIMP-2 variant and miR-142-3p target sequence is indicated with base sequence number of 6, and, specifically, was cut and inserted by using Nhe I and Hind III sites. The recombinant vector is shown in FIG. 1.

### Example 1-4. Confirmation of the Nerve Cells Specific Expression of Recombinant Vector In Vitro

Since miR142-3p is specifically expressed only in hemopoietic cells, the extent of the expression of AIMP2 variant was confirmed in specific cells in accordance with the knockdown of AIMP2 variant according to the expression of miR142-3p target sequence of the recombinant vector.

Specifically, there were group with no treatment of the recombinant vector (SHAM), void/control vector processed group (NC vector), single AIMP2 variant vector processed group (pscAAV_DX2) and group treated with the recombinant vector (pscAAV-DX2-miR142-3 pT). The concentration of all the vectors is in the unit of ug/ul and each group was treated with 2.5 ul (2.5 ug). In each of the treatment groups, treatments were made on the THP-1 cells strain (human leukemic monocyte cells) and SH-SY5Y cells strain (neuroblastoma) with confirmation of knockdown of AIMP2 variant. qPCR was executed by using the primers in Table 1 below (degeneration for 15 seconds and annealing and extension over 40 cycles under the temperature of 60° C. for 30 seconds).

TABLE 1

| AIMP2 | | SEQ |
|---|---|---|
| variant | Primer | ID NO |
| Forward | CTGGCCACGTGCAGGATTACGGGG | 8 |
| | (only human) | |
| Reverse | AAGTGAATCCCAGCTGATAG | 9 |
| | (only human) | |

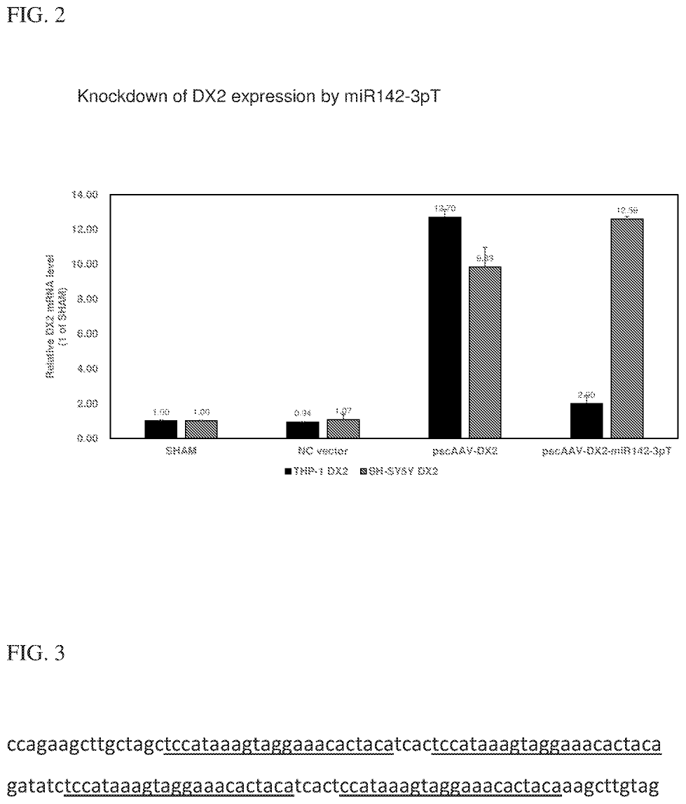

As a result, it was confirmed that AIMP2 variant is not expressed in the SHAM and NC vector groups. In addition, it was confirmed that there was expression in both the THP-1 cell strain and SH-SY5Y cell strain of the single AIMP2 variant vector processed group (pscAAV-DX2), thereby confirming that nerve cell-specific expression is not induced. On the other hand, it was confirmed that the AIMP2 variant is specifically expressed only in the SH-SY5Y cell strain in the group treated with the recombinant vector (FIG. 2).

### Example 1-5. Materials and Methods

### Example 1-5-1. qRT-PCR

Total RNA was isolated from spinal cord using TRIzol (Invitrogen, Waltham, MA, USA) according to the manufacturer's protocol. The extracted RNA was quantified by a spectrophotometer (ASP-2680, ACTgene, USA) for quantification. For making cDNA, a reverse transcription was performed using the SuperScript III First-Strand (Invitrogen) through manufacturer's protocol. The resulting cDNA was used for real-time PCR using SYBR green PCR master mix (ThermoFisher Scientific, USA). Expression data of the duplicated result were used for 2-ΔΔCt statistical analysis and GADPH expression was used for normalization.

### Example 1-5-2. MiR142-3p Inhibition Experiment

miR-142-3p inhibition on DX2 expression could be observed from x1 miR-142-3p target sequence. The HEK293 cells were transiently transfected with the x1, x2, and x3 repeat miR-142-3p target sequence vectors, and also with 100 pmol miR-142-3p using lipofectamine 2000 (Invitrogen, US), and then incubated for 48 hrs. The amount of DX2 mRNA was analyzed by PCR. miR 142-3p inhibition on DX2 expression was observed from Tseq x1 repeat miR 142-3p target seq (FIG. 4B).

### Example 1-6. Three (3) Types of Vectors Generated for Inhibition Effect of Core Binding Sequence

Tseq x1 contains 1 core binding sequence, Tseq x2 contains 2 core binding sequences, and Tseq x3 contains 3 core binding sequences (FIG. 4A).

miR142-3p (100 μmol) inhibition on DX2 expression was started to be observed from x1 repeat miR 142-3p target sequence. The HEK293 cells were transiently transfected with the x1, x2, and x3 repeat miR-142-3p T seq vectors, and also with 100 pmol miR-142-3p using lipofectamin 2000 (invitrogen, US), then incubated for 48 h. Amount of DX2 mRNA was analyzed by PCR. When the number of core binding sequence in miR142-3p target seq are increased, miR142-3p inhibition on DX2 expression was also increased. Tseq x3 core sequence containing vector showed significant inhibition (FIG. 4B).

### Example 1-7. Core Sequence Mutation

Using mouse B cell microarray data and mirSVR score of miR-142-3p target gene, core sequence was predicted. Four regions of core sequences were substituted as follows: (5′-AACACTAC-3′→5′-CCACTGCA-3′) (see FIG. 3 for original sequence and FIG. 4A for schematic drawing).

### Example 1-8. Core Binding Sequence is Important DX2 Inhibition

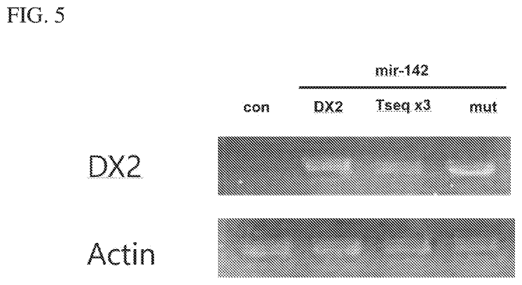

Four core sequences were substituted (FIG. 4A). The HEK293 cells were transiently transfected with the DX2-miR-142-3p T seq x3 repeated vector (Tseq3x) or with core sequence mutated vector (mut), and with 100 pmol miR-142-3p by using lipofectamin 2000 (Invitrogen, US), and then incubated for 48 hrs. Expression of DX2 mRNA was analyzed by PCR. Tseq x3 repeated vector which showed significant inhibition of DX2 (FIG. 4B) and DX2 construct were used as control. 100 pmol of miR142-3p treatment inhibited Tseq x3 vector significantly but DX2 and mut sequence were not inhibited (FIG. 5).

### Example 2. Treatment of RP (Retinitis Pigmentosa)

To explore the possibility of a safe and specific target for treating RP (Retinitis pigmentosa), AIMP2 was considered as a target for inhibiting cell death. AIMP2 accumulation leads to poly (ADP-ribose) polymerase-1 (PARP-1) activation and subsequent degeneration of neurons: DX2 compromises the pro-apoptotic activity of AIMP2 via the competitive inhibition of AIMP2 binding to PARP-1.

The enzyme poly (ADP-ribose) polymerase-1 (PARP-1) is the most studied member of the PARP family (also called ADPRT, PARS, ART), which comprises 17 members responsible for poly (ADP-ribose) (PAR) synthesis. Kim J H et al. (2014). The PARP enzymes are engaged in DNA repair, where they are activated by single and double DNA strand breaks. Their involvement in DNA repair appears to require the addition of PAR-chains (here referred to as PARylation) to the PARP enzyme itself or to other proteins involved in the repair process. Kim J H et al. (2014). In addition, abnormal activation of PARP-1 is involved in pathological processes such as stroke, trauma, diabetes and Parkinson's disease (Ko H S et al. (2005); Corti O. et al. (2003); Ko H S et al. (2010)), and in such or similar situations excessive accumulation of PAR may lead to a distinctive type of a cell death-parthanatos. Narne P et al. (2017); and Mao K et al. (2022). In this context, excessive PARylation could relocate apoptosis-inducing factor (AIF) from the mitochondria to the nucleus, with further rapid DNA defragmentation and cell death. Hone S J et al. (2004); Narne P et al. (2017); Mao K et al. (2022); Szabo C et al. (1996); Virag L et al. (1988). Excessive PARP activity is also seen in photoreceptor cell death in several rat and mouse models for RP, where it increases the level of PARylation in the degenerating photoreceptor cells. Sahaboglu A et al. (2017).

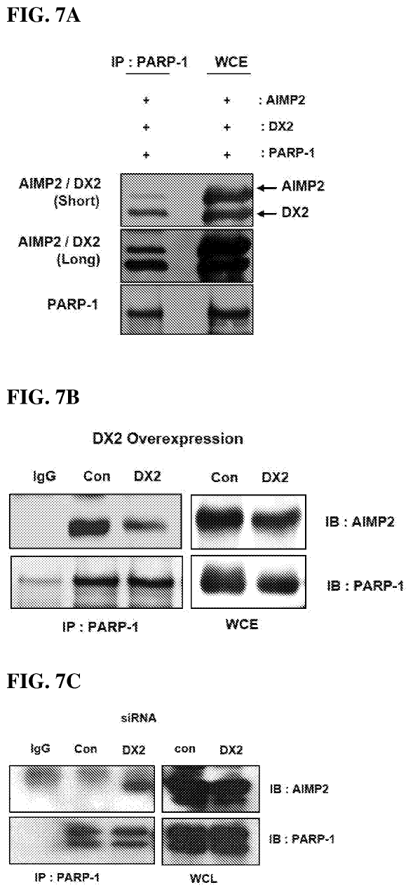

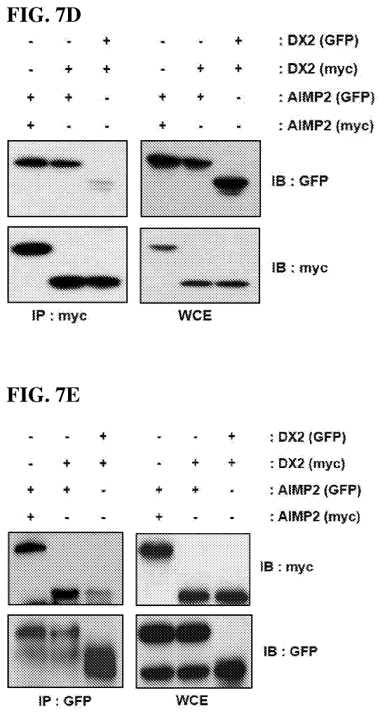

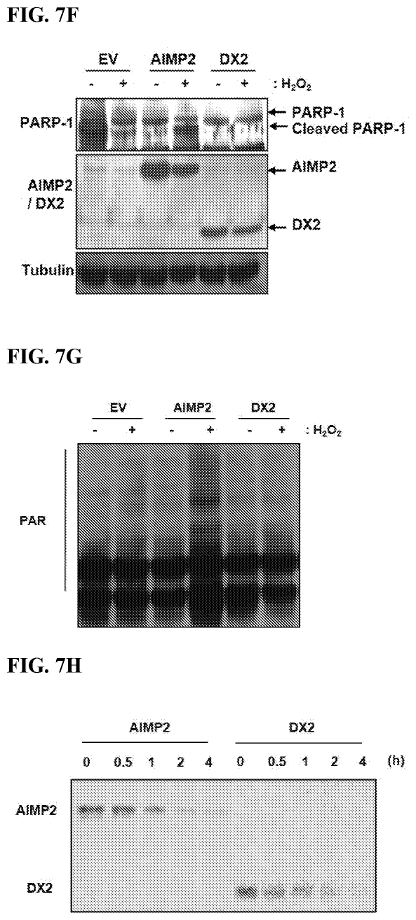

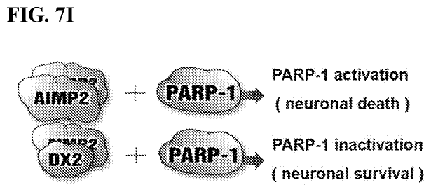

Thus, the efficacy of DX2 in inhibiting the function of AIMP2 in PARP-1 overactivation was tested. First, to investigate whether DX2 is a competitive inhibitor of AIMP2 and regulates neuronal cell death, SH-SY5Y cells were co-transfected with PARP-1, AIMP2, and DX2 expression plasmid and PARP-1 was immunoprecipitated. As shown FIG. 7A, DX2 binds to PARP-1 more strongly than AIMP2.

To determine whether the intracellular DX2 protein level affects the binding of PARP-1 and AIMP2, DX2 overexpression plasmid (FIG. 7B) or DX2 siRNA (FIG. 7C) were transfected into neuroblastoma SH-SY5Y cells prior to immunoprecipitation analysis. In this assay, SH-SY5Y cells were transfected with vehicle (Con) or DX2 expression (DX2) plasmid, incubated for 24 hours and lysed. Cell lysates were then incubated with protein agarose beads to immunoprecipitate PARP-1 bound AIMP2 and analyzed by immunoblot analysis (FIG. 7B). Similarly, DX2 siRNA (DX2) and control siRNA (Con) were transfected into SH-SY5Y cells, incubated for 48 hours, prior to immunoprecipitation and immunoblot analysis (FIG. 7C).

The amount of binding of AIMP2 to PARP-1 decreased in DX2 overexpressed cells (FIG. 7B), but in DX2 knockdown cells, the amount of binding of AIMP2 to PARP-1 is increased (FIG. 7C), suggesting that DX2 expression plays a critical role in the interaction between PARP1 and AIMP2.

To elucidate the mechanism by which DX2 regulates PARP-1 cleavage (e.g., PARP-1 activation) under conditions of oxidative stress, the binding affinities of AIMP2 and DX2 were assessed. AIMP2 and/or DX2 expressing vectors were introduced into HEK 293 cells. The cells were incubated for 24 hours, lysed, and immunoprecipitation was performed.

When AIMP2 was expressed alone, the formation of AIMP2 homodimers was observed. However, the amount of DX2 homodimers was significantly lower (FIGS. 7D-7E, third lane of left upper panel) compared with AIMP2-AIMP2 homodimers or AIMP2-DX2 heterodimer. Taken together, these observations suggest that DX2 prefers to bind to AIMP2 and inhibit AIMP2-induced cell death.

PARylation is a post-translational process, regulating biological events such as DNA damage response and apoptosis. Szabo C et al. (1996) and Virag 1 et al. (1998). PARP-1 is an enzyme that recognizes damaged DNA in the nucleus, forms PAR chains, and induces degradation of damaged proteins and cell death through the PARylation. To assess how AIMP2 or DX2 affects PARP-1 activation (e.g., PARP-1 cleavage) and PARylation under oxidative stress conditions, SH-SY5Y cells were transfected with vectors expressing empty vector (EV), AIMP2, or DX2 and then treated with hydrogen peroxide (H₂O₂) for 4 hrs. The cells were then lysed and the amount of activated PARP-1 protein level (FIG. 7F) and PARylation (FIG. 7G) was evaluated via Western blot analysisX.

AIMP2-transfected cells showed significantly increased cleavage of PARP-1 when compared to the expression seen in other transfected cells under oxidative stress conditions. However, PARP-1 cleavage was not observed in DX2-transfected cells (FIG. 7F). The PARylation of AIMP2 was increased in the presence of H2O2, but the PARlylation of DX2 was not altered (FIG. 7G).

To check the protein half-life between AIMP2 and DX2, a cycloheximide treated pulse-chase assay was performed. DX2 showed a similar stability to full length AIMP2 until 4 hours from stopping of de novo protein synthesis (FIG. 7H).

Thus, these results indicate that DX2 is a critical factor for neuronal cell viability and DX2 overexpression can reduce neuronal cell death in neurodegenerative diseases.

As an observation, when DX2 is insufficiently expressed, AIMP2 forms homodimers, interacts with PARP-1, activates PARP-1, and induces neuronal cell death. However, since DX2 has a significantly higher binding affinity to PARP-1 than AIMP2, the binding affinity between AIMP2 and PARP-1 was decreased in DX2 expressing cells, thereby leading to the inhibition of PARP-1 activity and the reduction in neuronal cell death. Thus, DX2 is a main regulatory protein in AIMP2-induced PARP-1 activation and cell death (FIG. 7I).

Taken together, DX2 compromises the pro-apoptotic activity of AIMP2 via the competitive inhibition of AIMP2 binding to PARP-1.

### Example 3. DX2 Attenuates H2O2-Induced Neuronal Cell Death

Based on the results above that DX2 is an inhibitory molecule of oxidative stress-induced PARP-1 cleavage, determination of whether DX2 suppresses cell death in the neuronal cells was further investigated. To check the cell viability in AIMP2 and DX2 overexpression conditions, an AIMP2 or DX2 expression plasmid was introduced in N2A cells (Neuro-2a, mouse neuroblastoma cell line) and the transfected cells were incubated with 400 μM H₂O₂ for 4 hours. MTT analysis was performed to determine the level of cell death.

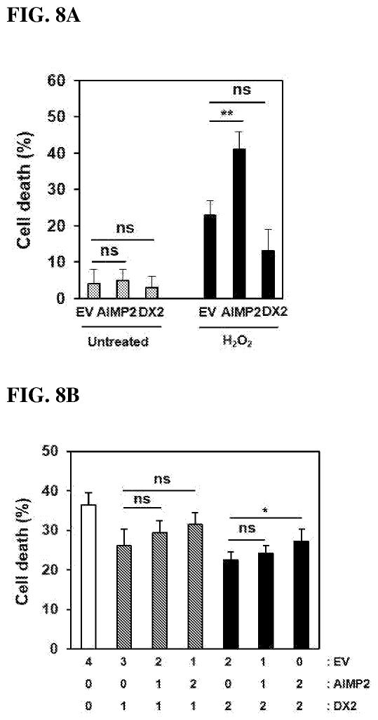

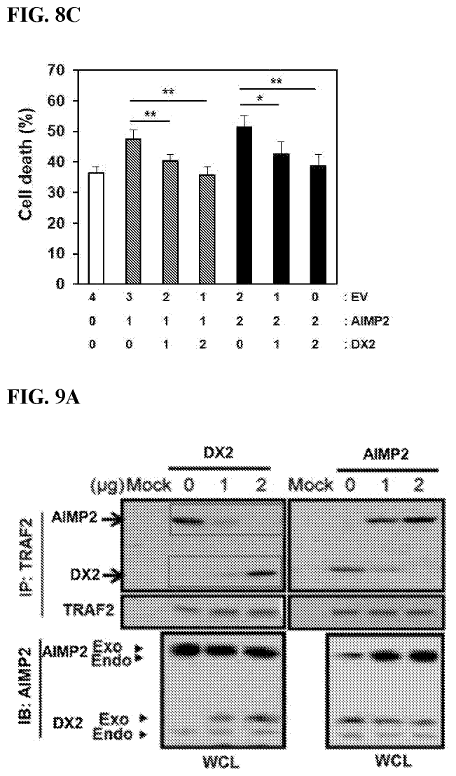

In normal conditions, the overexpression of AIMP2 or DX2 did not affect cell viability; however, in H₂O₂-treated conditions, cell death was significantly increased by the overexpression of AIMP2 and reduced by the overexpression of DX2 (FIG. 8A).

To test whether DX2 or AIMP2 controls cell viability in damage-induced cell death conditions, SH-SY5Y cells were co-transfected with EV, AIMP2 and DX2 expression plasmid and treated with 400 μM H₂O₂ for 4 hours. A MTT analysis was performed to determine cell death. Even when the expression of AIMP2 was increased (1 ug to 2 ug), a 1 ug transfection of DX2 suppressed AIMP2/H₂O₂-induced cell death to a level similar to that of 2 μg of DX2 (FIG. 8B). In contrast, AIMP2 expression level was not able to significantly affect neuronal cell death under DX2-expressing cellular conditions (1 ug to 2 ug) (FIG. 8B) and DX2 expressing cells have significantly reduced cell death compared to control cells under oxidative stress conditions (FIG. 8C). Taken together, the anti-apoptotic effect of DX2 seems to be much stronger than apoptosis induction by AIMP2 in the co-existent condition of the two-proteins.

### Example 4. TRAF Dependent Pathway: DX2 Compromises the TNF-α-Dependent Pro-Apoptotic Activity of AIMP2 Via the Competitive Inhibition of AIMP2 Binding to TRAF2

DX2 compromises the TNF-α-dependent pro-apoptotic activity of AIMP2 via the competitive inhibition of AIMP2 binding to TRAF2.

Ocular inflammation is a common cause of visual impairment. Olivares-Gonzalez L et al. (2021). It is considered to be a chronic inflammation with sustained activation of glial cells (microglia, astrocytes) and recruitment of other immune cells into the nervous tissue (brain, spinal cord, or retina). Streit W J (2004). Microglia cells, the resident innate immune cells in the retina and other nervous tissue, are potential cellular regulators of inflammation. Karlstetter M et al. (2015). Activated microglia and macrophages secrete pro-inflammatory mediators such as TNF-alpha and t on retinal cells (e.g., photoreceptor cells). Murakami Y et al. (2019). In the eye, TNF-a appears to participate in the pathogenesis of inflammatory, edematous, neovascular, and neurodegenerative diseases. Rodrigues E B et al. (2009).

In TNF-α signaling, TRAF2 plays a crucial role in cell death. When the TNF-α signaling is activated, TRADD (TNF Receptor Associated Death Domain) and TRAF2 bind to TNFR1, which is a TNF-α receptor and then, the activated complexes mediate IκB kinase (IKK) activation. Upon which caspase-8 is inactivated, TRAF2 is released from the TRADD complex, and the released TRAF2 is ubiquitinated by cIAP, which is a E3 ubiquitin ligase, promoting cell death. AIMP2 promotes the ubiquitination of TRAF2 by cIAP and stimulates cell death. Choi J W et al. (2009).

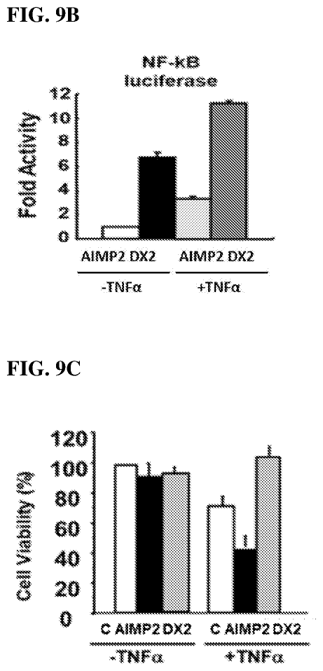

To understand how DX2 compromises the apoptotic activity of TNF-a signaling, we evaluated the ability of DX2 to compete with AIMP2 for the interaction with TRAF2 and to affect NF-kB-dependent transcriptional activity. For the co-immunoprecipitation assay, HEK293 cells were transfected with 0, 1 or 2 μg of Myc-tagged DX2 or AIMP2 expression plasmid (FIG. 9A). The expression of endogenous (Endo) and exogenous (Exo) AIMP2 and DX2 was confirmed by immunoblotting of whole cell lysates (WCL) with anti-AIMP2 antibody. TRAF2 was immunoprecipitated with its specific antibody and AIMP2 or DX2 bound to TRAF2 were detected with anti-Myc antibody.

To test the NF-kB-dependent transcriptional activity, HEK293 cells were transfected with NF-kB-luciferase vector and the cells were selected by G418 (1 mg/ml) for 1 week to establish the stable expressing cells. After the selection of cells survived, the cells were transfected with the pcDNA3.1 empty vector (as the control) or pcDNA 3.1 expression vector encoding the AIMP2 or DX2. Sixteen hours later, the transfected cells were treated with TNF-α (20 ng/ml) for 12 hours. The cells were harvested, and cell lysates were prepared for quantitation of luciferase using a luciferase assay kit following the manufacturer's protocol (Promega).

As the expression of DX2 was increased, the interaction of AIMP2 with TRAF2 was decreased. Conversely, the introduction of AIMP2 inhibited the interaction of DX2 with TRAF2 in a dose-dependent manner (FIG. 9A). Increased NF-kB activity suppressed the pro-apoptotic signal of TNF-α. As expected, DX2 increased the NF-kB activity, whereas AIMP2 gave the opposite effect (FIG. 9B). DX2 overexpression abolished the TNF-α induced cell death (FIG. 9C).

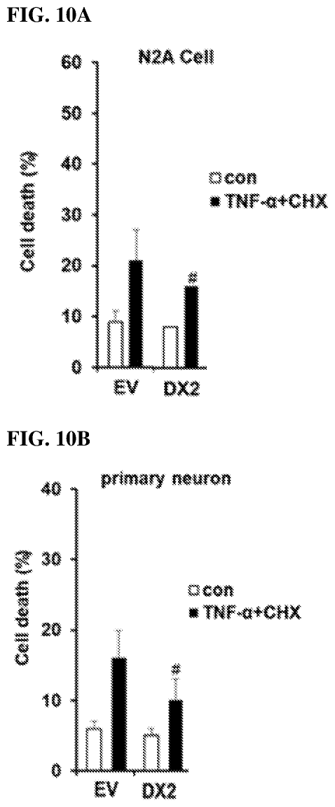

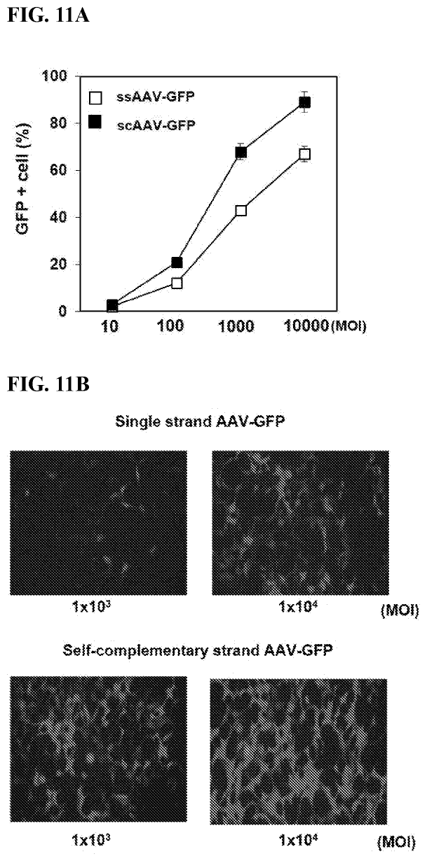

Next, to assess whether DX2 suppresses TNF-α dependent neuronal cell death, N2A and mouse primary neuron cells were transfected with EV (empty vector) or DX2 expression plasmid. The transfected cells were incubated in the presence or absence of TNF-α (20 ng/ml)+cycloheximide (CHX, 10 uM) for 6 h. Cell viability was then evaluated using a MTT assay. As shown in FIGS. 10A and 10B, DX2 overexpression did not affect cell viability of N2A or mouse primary neuron cells under normal conditions (con); however, upon TNF-α treatment, the cell death of DX2 transfected cells (DX2) were significantly reduced compared to the EV (empty vector) transfected cells.

Taken together, DX2 competes with AIMP2 and interacts with TRAF2 more strongly than AIMP2, thus inhibiting AIMP2-stimulated neuronal cell apoptosis in TNF-α-activated conditions.

### Example 5. Design of AAV2-DX2: DX2-Coding Self-Complementary AAV2

As it is commonly difficult to treat retinal degenerative disease (RDD) using usual pharmacological methods, more effective strategies are being sought. Among these alternatives, gene therapy can be a strategy to access RDD and treat diseases, such as RP with minimal invasiveness of subretinal micro-injection. Christine C W et al. (2009); Eberling J L et al. (2008); Castle M J et al. (2020).

AAV vectors are efficient shuttles that are able to deliver a transgene to the retinal cells, with their efficiently transducible character to non-dividing retinal cells. AAV is also appreciated as a suitable gene delivery approach because of its relative safety due to a lack of pathogenicity. Wang D et al. (2019). Here, the potential of an adeno-associated virus serotype 2 (AAV2) delivery system to effectively deliver DX2, a therapeutic gene, to RDD patients is investigated.

To determine which viral system to use between single-strand AAV (ssAAV) and self-complementary AAV (scAAV), SH-SY5Y cells were infected with 10, 100, 1000 or 104 MOI (Multiplicity of infection) of ssAAV-GFP (Green Fluorescent Protein) or scAAV-GFP. After 48 hours, GFP expression was measured by microscopy. FIG. 11A represents a percentage of GFP positive cells and FIG. 11B shows a microscopy image of GFP expressing cell. In FIGS. 11A and 11B, scAAV was more effective in virus infection rates when both viruses were used to treat SH-SY5Y at different concentrations.

### Example 6. AAV2-DX2 Transduction Suppresses Neuronal Death-Associated Cellular Signaling

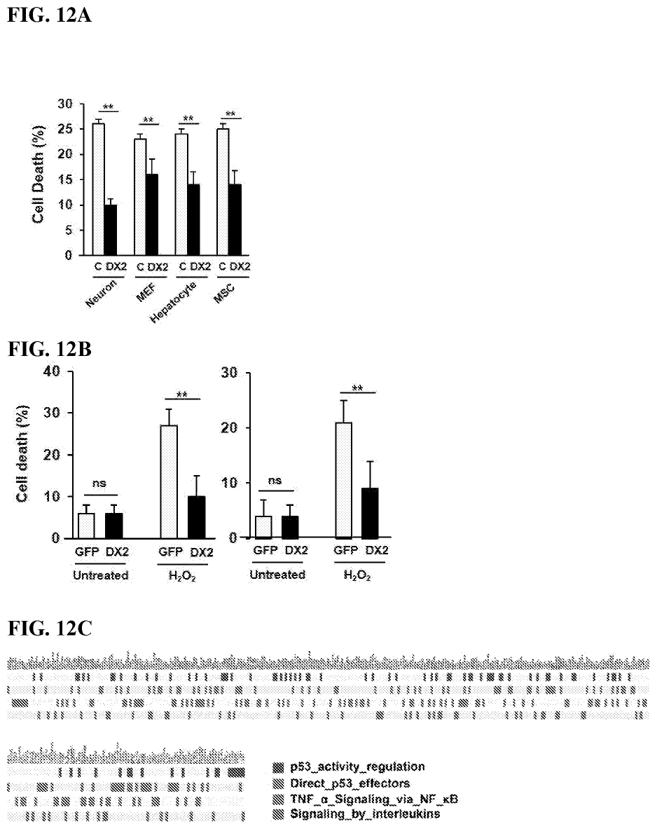

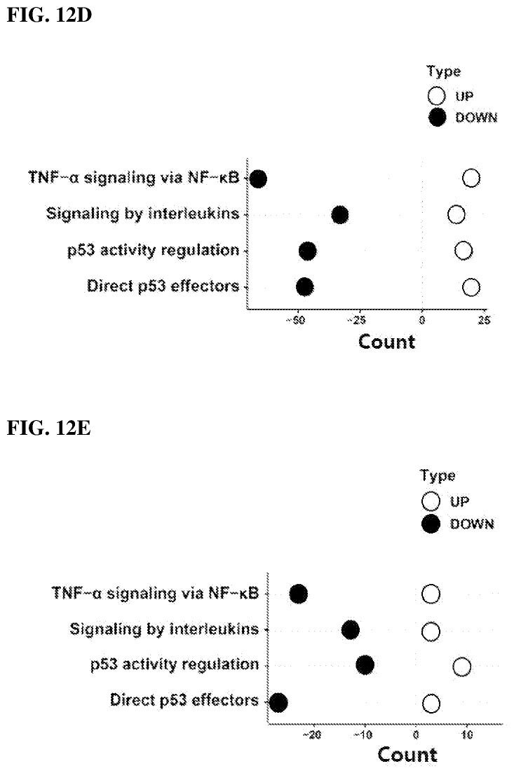

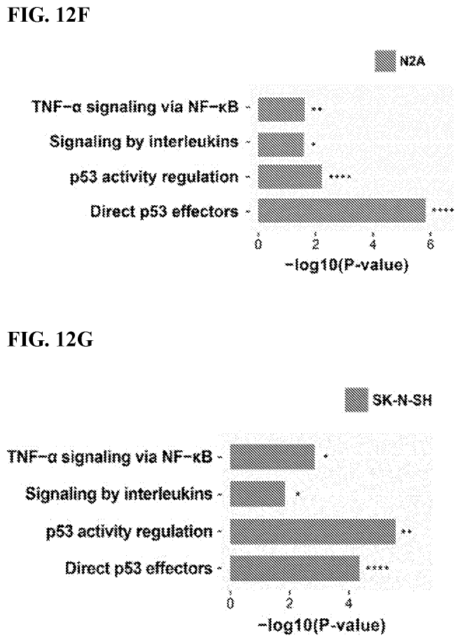

To investigate whether AAV2-DX2 compromises the H₂O₂ induced cytotoxic effects in the MEF (Mouse embryonic fibroblasts), hepatocytes, MSC (Mesenchymal stem cells) as well as in the primary neurons (Neuron), the cells were infected with 104 MOI of AAV-DX2 (AAV2-DX2) for 48 hours. Then, the transduced cells were treated 400 μM H2O2 for 4 hours to induce cell death and cell viability was measured by MTT analysis (FIG. 12A).

AAV2-DX2-transduced cells (DX2) commonly showed significant decreases in cell death when compared with AAV-GFP-transduced cells (GFP) after H2O2-treatment (FIG. 12A), implying that AAV2-DX2 is an effective anti-apoptotic agent. Interestingly, under normal conditions, cell viability was not affected by the presence or absence of AAV2-DX2 (FIG. 12B). Therefore, DX2 functions only when a stressor induces apoptotic conditions.

To investigate how DX2 overexpression changes cellular signaling in neuronal cells, RNA sequencing was conducted on neuronal cells after induction DX2 gene by an AAV delivery system. SK-N-SH neuroblastoma cells were infected with either scAAV2-GFP or AAV2-DX2. Forty-eight hours following infection, the cells were harvested, RNA was isolated and RNA sequencing was performed. DX2 was predicted to be involved in suppression of p53-associated cell death pathway and TNF-α or interleukin-related signaling (FIG. 12C). The gene set was analyzed based on two cell types, SK-N-SH (FIG. 12D) and N2A (FIG. 12E), to assess differently expressed genes (DEGs) that were categorized into signaling pathways or in ontology database. DX2 appeared to reduce the inflammatory or apoptotic signaling in both N2A (FIG. 12D) and SK-N-SH cells (FIG. 12E), displaying an abundancy of downregulation of related genes. The statistical significance was visualized in the distribution of a p-value graph for all of the pathways post DX2 overexpression (FIGS. 12F-12G).

### Example 7. DX2 Increases 661 W Cell Viability, a Retinal Photoreceptor and Ganglion Precursor-Like Cell Line

To investigate whether DX2 can rescue retinal photoreceptor and ganglion neurons from ROS induced cell death, as does in other neuronal cells described in above paragraphs, we performed a cell viability test on cells infected with AAV-DX2 and non-infected cells treated with varying concentrations of H₂O₂. The results showed that cells infected with AAV-DX2 exhibited greater resistance to H₂O₂ concentrations and remained viable compared to non-infected cells.

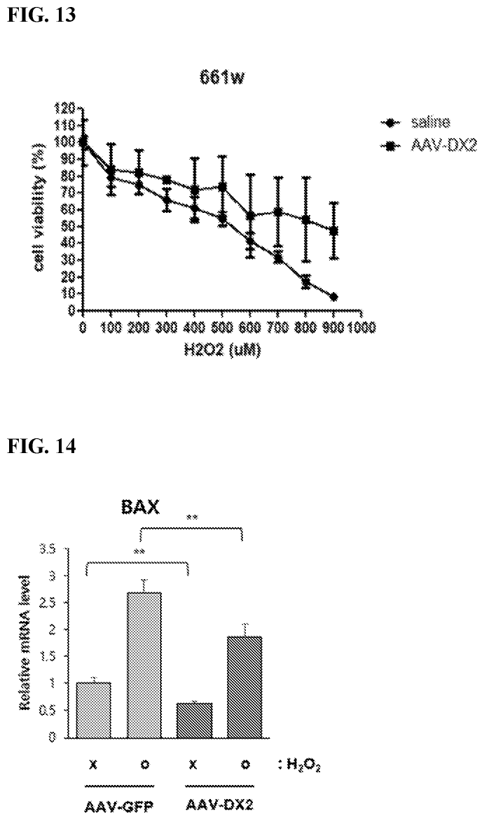

FIG. 13 shows that DX2 increased retinal photoreceptor neuronal cell viability. 661 W cells were seeded at 5×10\hat{ }4 cells per well in a 96-well plate, followed by infection with AAV-DX2 virus at an 5000MOI. After 48 hours, cells were treated with varying concentrations of H2O2. Cell viability was assessed 24 hours later using the MTT assay.

To further examine the anti-apoptotic effects of AAV-DX2, 661 W cells were infected with AAV-DX2 then cell death was induced with H₂O₂ treatment. the mRNA levels of the pro-apoptotic marker BAX was assessed. The results showed that in cells infected with the control virus, AAV-GFP, BAX levels increased more than 2.5-fold upon H₂O₂ treatment compared to untreated cells. However, in cells treated with AAV-DX2, BAX levels were significantly reduced when treated with H₂O₂ compared to the AAV-GFP treated group.

FIG. 14 shows that DX2 reduces the levels of BAX induced by H2O2. 661 W cells were infected with either AAV-GFP or AAV-DX2 at an MOI of 5,000 each. After 72 hours, cells were treated with 2 mM H₂O₂ for 6 hours, and then the RNA levels of BAX were assessed.

### Example 8. DX2 Increases 67LR Expression Levels in Retinal Neurons

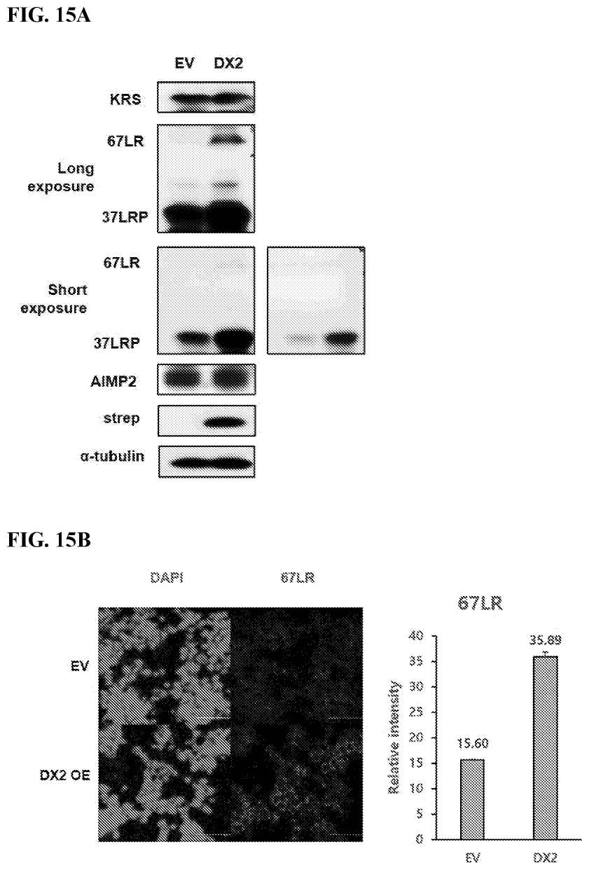

To further examine the anti-apoptotic signaling pathway of DX2, we investigate the regulatory role of DX2 in 67LR. DX2 was overexpressed in 661 W, a retinal photoreceptor and ganglion precursor-like cell line and examined the changes in 67LR expression. Upon DX2 overexpression, an increase in both 67LR and 37LRP expression was observed (FIG. 15A). This observation was further supported when we performed immunofluorescence staining of 67LR in 661 W cells under the same experimental conditions. The cells overexpressing DX2 exhibited a fluorescence intensity approximately 2.5 times stronger than that of the non-overexpressing cells (FIG. 15B).

Next, both AIMP2 and DX2 were overexpressed, followed by immunoprecipitation with KRS, to assess the binding affinity of DX2 and KRS. The results showed that both AIMP2 and DX2 exhibited similar binding affinity to KRS.

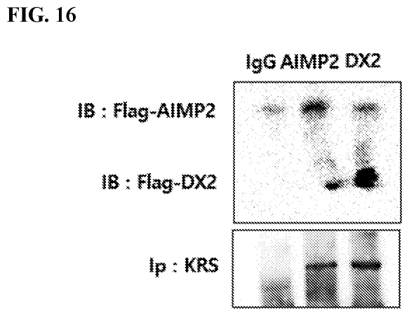

FIG. 16 shows that DX2 binds to KRS similarly to AIMP2. Cells transfected with 2 μg of Flag-tag-AIMP2 and Flag-tag-DX2 were subjected to cell lysis using NP-40 lysis buffer. Subsequently, a mixture of 15 μl agarose A/G plus beads and KRS antibody was used for the pull-down assay.

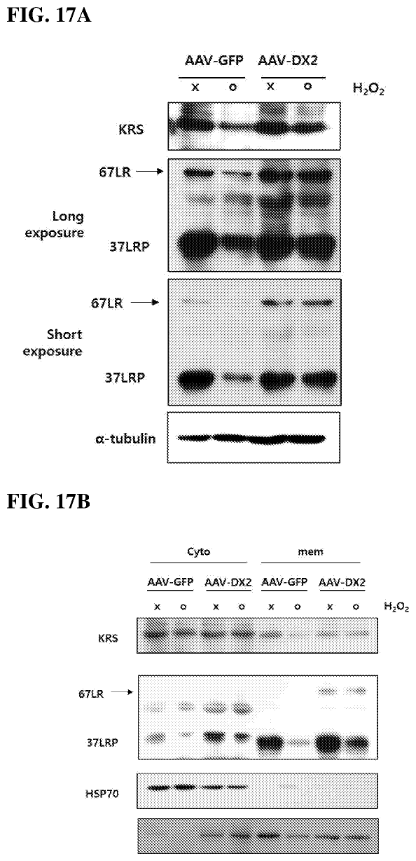

As previously reported, H2O2-induced ROS stress causes a decrease in 67LR, which weakens cellular survival signaling and triggers cell death. Yan X et al. (2017). In conditions where retinal cells are dying due to ROS, we examined the changes in KRS and the effects of DX2 by infecting 661 W cells with AAV-DX2. When treated with the control virus, AAV-GFP, a reduction in 67LR levels was observed due to H₂O₂ treatment, and KRS levels also decreased. However, when treated with AAV-DX2, there was no decrease in 67LR levels due to H2O2, and no changes in KRS levels were observed (FIG. 17A).

To determine where the changes in KRS and 67LR had the most significant impact, either in the cytosol or membrane, we analyzed the cells after fractionation. The results showed that the decrease in KRS levels caused by H₂O₂ was not in the cytosolic form, but rather in the membrane form, and the increase in 67LR levels also occurred in the membrane (FIG. 17B). Taken together, these results implies that DX2 binds to KRS, resulting in an increase of the membrane form of KRS and consequently the expression of 67 LR expression and thereby prevents the neuronal cell from anoikis.

### Example 9. In Vivo Studies Evaluating the Effect of AAV2-DX2 on RDD

To understand the potential efficacy to Retinal degenerative diseases patients, the preventive effect and curative effect of AAV2-DX2 was evaluated in three RDD animal models in mouse and rabbit, i.e., laser-induced choroidal neovascularization, Mdm1−/− (CRISPR/Cas9 KO) and Sodium iodate induced retinal degeneration model. Overall, AAV2-DX2 was determined to be effective in both the prevention and therapeutic settings. These studies are summarized below.

### Example 9-1. AAV2-DX2 Shows Preventive Effect of DX2 in the Mouse Mdm1−/− (CRISPR/Cas9 KO) Model

Mouse double minute 1 (Mdm1) might be involved in the function and structure of centrioles and degeneration. Son et al. demonstrated that Mdm1 protein is localized at the connecting cilium (CC) of photoreceptor cells in the retina. Depletion of the Mdm1 transcript may underlie the mechanism leading to onset progressive retinal degeneration.

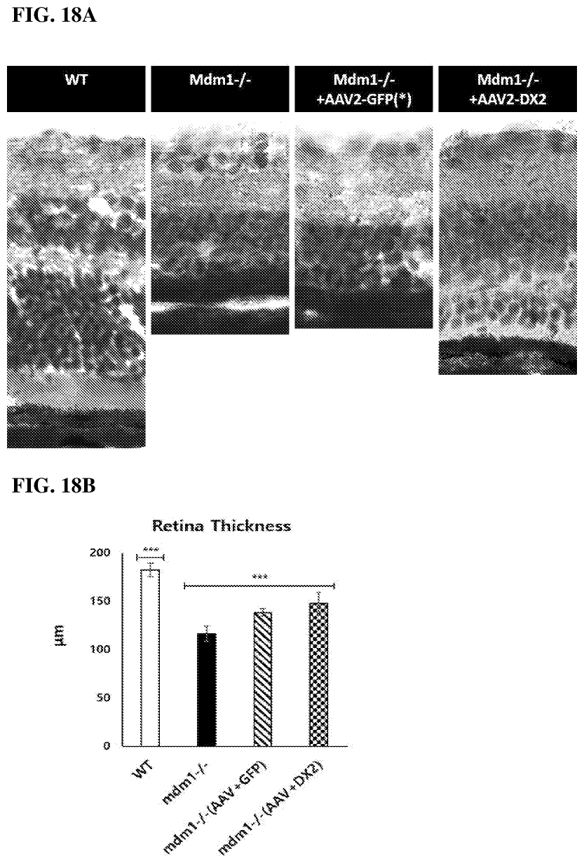

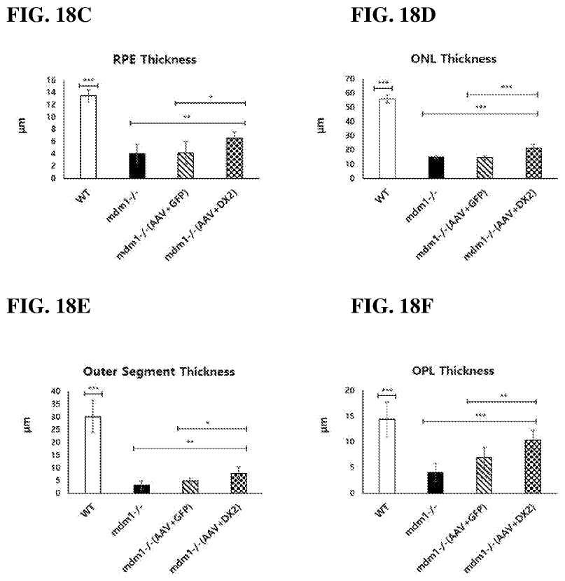

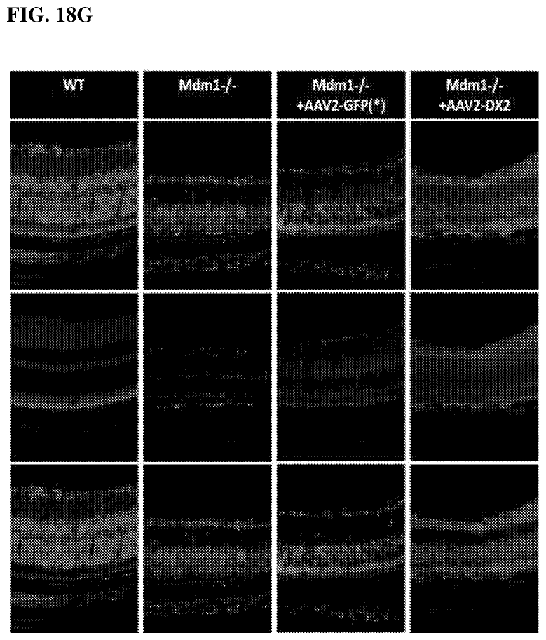

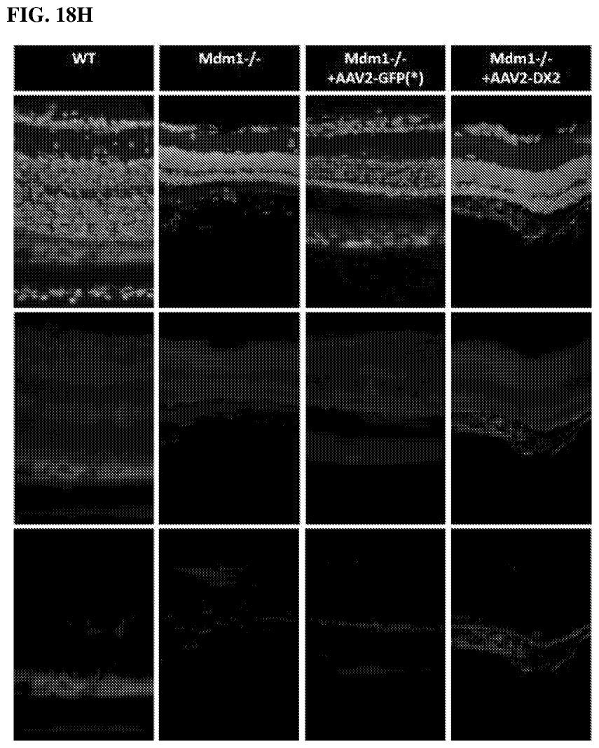

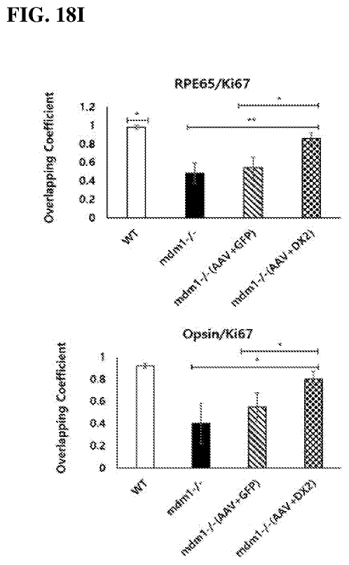

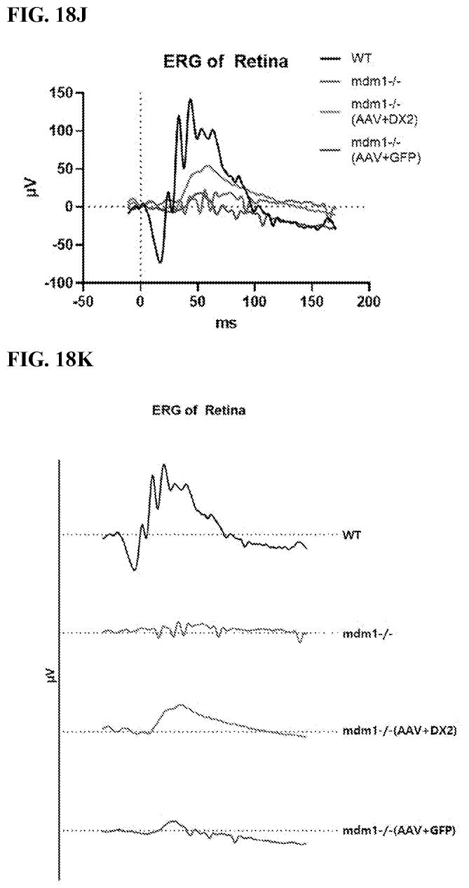

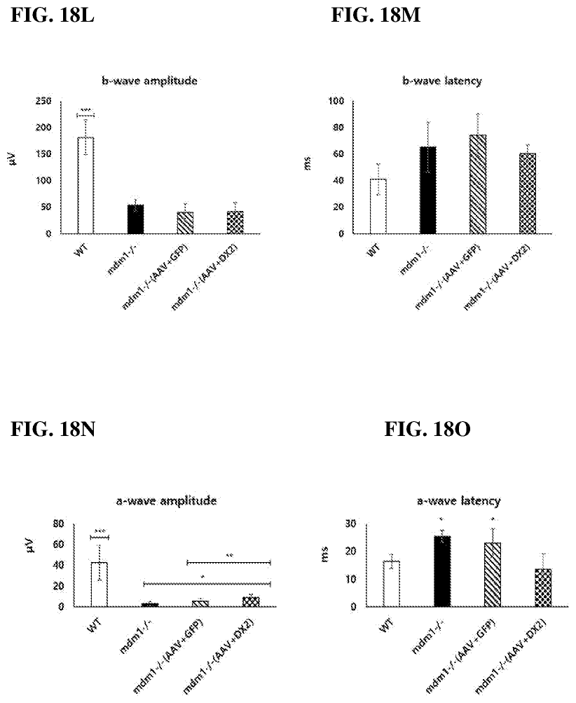

Mdm1−/− mouse was generated? using CRISPR/Cas9 and the mice exhibit retina degeneration and abnormal eye electrophysiology³⁹. Transduction of AAV-DX2 in retina resulted in the recovery of the total neural retina thickness (FIG. 18B). transduction of DX2 showed thicker RPE layer (FIG. 18C) and the photoreceptor outer segment layer (FIG. 18D), implying that DX2 expression prevents RPE degeneration and photoreceptor cilia degradation. Transduction of DX2 also showed thicker Outer Nuclear Layer of photoreceptors (FIG. 18E) and Outer Plexiform Layer (FIG. 18F), indicating that DX2 expression reduces photoreceptor degeneration.

FIG. 18G shows integrity and proliferation of RPE (Retinal Pigment Epithelial). Transduction of DX2 gene resulted in the recovery of RPE integrity by activating proliferation of RPE. FIG. 18H shows PR (Photoreceptor) recovery. Transduction of DX2 gene resulted in recovery of PR population by activating proliferation of PR.

FIG. 18I shows cellular proliferation of RPE and PR. Ki67 expression was measured to analyze proliferation in RPE and photoreceptor layers. Proliferation in RPE (left panel) and photoreceptor outer segment layer (right panel) was significantly higher in the AAV2-DX2 transfected sample.

FIGS. 18J-180 show functional recovery of retina. AAV2-DX2 transduction showed increased a-wave amplitude (FIG. 18L) and reduced latency (FIG. 18M) compared to the RDD model (mdm1−/−) or the negative control (mdm1−/−+AAV-GFP), indicating that DX2 expression reduces damage of photoreceptors' electrophysiological function and visual acuity. AAV2-DX2 transfected sample did not show a change in b-wave amplitude (FIG. 18N) but reduced the latency (FIG. 18O) compared to the RDD model (mdm1−/−), which suggests that DX2 affects only the RPE and photoreceptor, but not the bipolar cells (post-photoreceptors neurons).

Electroretinography of AAV2-DX2 transduced sample showed increased regaining of normal ERG graph format (FIGS. 18J and 18K). AAV2-DX2 transfected sample showed slightly increased a-wave amplitude (FIG. 18L) and reduced latency (FIG. 18M) than the dry AMD (mdm1−/−) indicating that the DX2 expression reduced the damage of photoreceptors' electrophysiological function. The AAV2-DX2 transduced sample did not show a change b-wave amplitude (FIG. 18N) but reduced the latency (FIG. 18O) than in the RDD model (mdm1−/−).

Survival effect of DX2 on retinal degeneration and anti-apoptotic efficacy of DX2 delivered by adeno-associated virus (AAV) into the retina of rabbit via SR and IV injection.

### Example 9-2. Materials and Methods

### 1. Animal Experiments (Knockout Mouse)

For the retinal degeneration mouse model experiment, Mdm1−/− (CRISPR/Cas9 KO) mouse was generated to present progressive photoreceptor and RPE degeneration. The animals were housed in individual cages under specific pathogen-free conditions and a constant environment condition (21° C.-23° C. temperature, 50-60% humidity and 12-h light/dark cycle) in the animal facility. AAV2-DX2 and Negative control (AAV2-GFP) injection at Sub-retinal space at 3 weeks old. Histological measurements and functional recovery of retina were performed at 3-months old. Three-week-old Mdm1−/−mice were injected at subretinal space by trans-scleral injection to minimize retinal wound with AAV2-DX2/AAV2-GFP in a volume of 4 μl using a 38G sterile micro-tip needle (INCYTO, KR). AAV2-GFP/DX2 was injected into the same animal. AAV2-GFP was injected at OS (left). AAV2-DX2 was injected at OD (right).

### 2. Electrophysiological Function Evaluation

Three-week-old Mdm1−/−mice were injected at subretinal space by trans-scleral injection to minimize retinal wound with AAV2-DX2/AAV2-GFP in a volume of 4 μl using a 38G sterile Three-month-old mice were used. For final measurements, WT (n=11), mdm1−/− (n=6), mdm1−/− (AAV2+GFP) (n=6) and mdm1−/− (AAV2+DX2) (n=7) were evaluated. For electroretinogram for photoreceptor function evaluation, the OcuScience® HMSERG was used. Mice were induced to anesthesia with Avertin (1%) and anesthesia was maintained with 3% isoflurane inhalation and put on heating pad to maintain their physiological condition. A drop of 2% hypromellose solution drop was placed on the Rodent Contact Lens with Silver-embedded Thread Electrode to keep contact with the cornea and to keep it moistened. Mice were placed under the 76 mm diameter Ganzfeld dome for darkness and uniform illumination of the eyes. Measurements were performed under ISCEV-Extended full-field ERG standards protocols. The data were analyzed using ERGVIEW and the combined standard Rod&Cone response value was selected to analyze with a flash intensity of 3000mcd.s/m2, 0.10 Hz. A-wave analysis was performed for photoreceptor cell function. B-wave analysis was performed for bipolar and horizontal cell function. Amplitude and latency values for a-wave and b-wave were analyzed.

### 3. Histological Analysis

All mice were euthanatized after ERG and their eyeballs were harvested. The eyeballs were fixed in 4% PFA overnight at 4° C. Eyeballs were dehydrated at 30% sucrose and embedded with the OCT compound for tissue cryosection. All retina cryosection samples were acquired from the optic nerve containing section with 10 μm thickness. Retina cryosections were analyzed. H&E was used for layer thickness analysis. Layer thickness analysis was performed with Leica LAS program. Immunofluorescence was used for RPE65 and Opsin expression, and Proliferation evaluation (Ki67). Immunofluorescence ROI set and overlapping coefficient measurements were measured with Image J.

### 4. Statistical Analysis

The Student's t-test was used for primary analysis. P-values were compared with mdm1−/− (AAV2+DX2). For recovery evaluation, P values were compared to WT. For full data, Levene's Homogeneity of Variance test, ANOVA tests, and post-hoc (Dunnett (T3), Tukey HSD) evaluation were performed with IBM SPSS statistics 23.

### Example 10. AAV2-DX2 Show Preventive Potency of DX2 in the Laser-Induced Choroidal Neovascularization (CNV) Mouse Model

### Example 10-1. AAV2-DX2 Treated Mice Attenuates Laser-Induced Choroidal Neovascularization

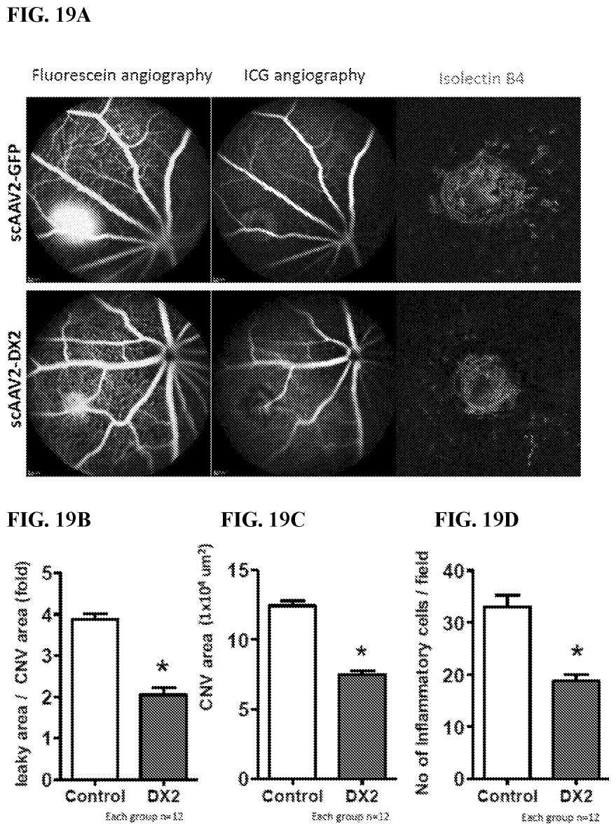

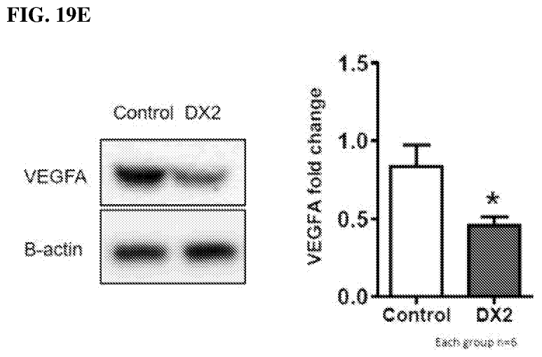

Laser-induced choroidal neovascularization (CNV) model is a widely used animal model for retinal degenerative disease (RDD). In this model, laser is used to disrupt Bruch's membrane, which allows the underlying choroidal vessels to penetrate and grow into the space underneath the pigment epithelium. Subretinal injection of scAAV2-GFP (control) or AAV2-DX2 to 5-wk-old male C57BL/6 mice (n=12) were conducted (Day 0). Twenty-one days after injection, CNV was produced by laser photocoagulation (Day 21). Fourteen days after laser treatment, Fluorescein angiography and ICG angiography performed (Day 35). Next day, mice were killed and choroidal flat mounts generated and stained with (FITC)-conjugated isolectin B4 (FIG. 19A) (Day 36). Vascular leakage caused by the new blood vessel formation was clearly observed at the laser-induced photocoagulation site by fluorescein angiography. ICG angiography is used to acquire an angiogram of the choroid. Flat mounts were used to evaluate the presence and area of clearly demarcated isolectin positive CNV. DX2 injection showed decreased vascular leakage compared to GFP injection control in fluorescein angiography (FIG. 19A). Similarly, DX2 injected mice showed reduced CNV area compared to GFP injected mice in ICG angiography (FIG. 19A). Also, choroid flat mounts stained with isolectin-B4 demonstrates significant reduction in CNV formation areas in DX2 injected mice (FIG. 19A).

The ratio of leaky area to CNV area were estimated by measuring the total hyperfluorescent area using fluorescein angiography (FA) and the CNV area using ICGA (FIG. 19B). The mean CNV area at Bruch's membrane using isolectin B4 staining were also significantly smaller in DX2 injected mice compared with that in GFP controls (n=12) (FIG. 19C). Based on this result, DX2 had preventive effect in the CNV mouse model. Inflammatory cells, in particular (macrophages), have been histologically demonstrated near/within degenerative lesions, including areas of Bruch membrane breakdown, RPE atrophy, and CNV. (Macrophages) in CNV lesions have been shown to secrete proangiogenic factors such as VEGF and proinflammatory cytokines such as TNF. In FIG. 19D, the number of inflammatory cells of DX2 injected mice are significantly smaller compared to GFP control CNV cell. An excessive amount of vascular endothelial growth factor (VEGF) triggers the growth and leakage of abnormal blood vessels under the macular, resulting in irreversible loss of central vision. In this context, many efforts have been made toward the development of anti-angiogenic therapies targeting VEGF for the treatment of retinal degenerative disease. These drugs have been shown to slow the progression of RDD, and in some cases, improve vision acuity by suppressing angiogenesis. Here, we treated DX2 in advance of laser-induced choroidal neovascularization mice and compared GFP treated mice for VEGF expression (n=6) (FIG. 19E). DX2 treated mice showed less VEGF expression compared to GFP treated mice. This data also showed preventive effect of DX2 on CNV model mouse.

### Example 10-2. Materials and Method

### 1. Animal Experiments (Mouse)

The animals were housed in individual cages under specific pathogen-free conditions and a constant environment condition (21° C.-23° C. temperature, 50-60% humidity and 12-h light/dark cycle) in the animal facility. The mouse Ocular sinister (OS, left eye) in each group treated AAV-GFP and Ocular Dexter (OD, right eye) treated AAV-DX2. (Injection: 5×108 vg). After sub-retinal injection 1 week and 10 weeks, using laser photo-coagulator, RPE layer of eye fundus induce laser to make 3 weeks-, 3 months-retinal degenerative disease model, respectively. After 2 weeks, 2 months of laser treatment, the mouse eyeball is isolated.

### 2. Sub-Retinal Injection Protocol.

Anesthetize the rodent. Use intraperitoneal injections of 100 mg/ml ketamine and 10 mg/ml xylazine (20 μl/10 g body weight) over isofluorane inhalation. Ensure that the animal is deeply anesthetized by pinching one of its paws. If it flinches, wait several more minutes and try again before beginning the sub-retinal injection. Position the rodent onto its side so that the eye that will be injected is facing the ceiling. Under a dissecting microscope gently stretch the skin so the eye pops slightly up out of the socket (temporary proptosis) and becomes more accessible by holding its head with two fingers just above the ear and by its jaw and gently stretch the skin parallel to the eyelids so that the eye pops slightly up out of the socket. 38G sterile micro-tip needle (INCYTO, KR), make a hole immediately below the limbus and at an angle to avoid touching the lens with the needle. Retract the disposable sharp needle from the eye while maintaining the grip on the head. After either mounting the pre-loaded syringe with a blunt needle on a micromanipulator or holding it by hand, insert the tip of the syringe with the blunt needle through the hole, taking care again not to touch the lens and gently push it through the eye very gently until feeling resistance. Keeping all movements to a minimum, carefully inject the viruses slowly into the sub-retinal space. AAV2-GFP was injected at OS (left). AAV2-DX2 was injected at OD (right). Retract the syringe slowly. Apply eye moisturizing drops to keep the eye hydrated. Continue to monitor the animal until it regains sternal recumbency.

### 3. Mouse Laser-Induced CNV Model Protocol.

Before the induced Laser to mice, Position the laser and slit lamp where it can be easily accessible. Turn on laser and set to pre-determined parameters. Anesthetize the rodent. Use intraperitoneal injections of 100 mg/ml ketamine and 10 mg/ml xylazine (20 μl/10 g body weight) over isofluorane inhalation. Roll mouse on its side and place a drop (approximately 30 μl) of tetracaine hydrochloride into each eye for topical anesthesia. Wait 2 min for solution to take effect. Repeat previous step with one drop of topical tropicamide for pupillary dilation. Alternatively, use phenylephrine hydrochloride (2.5%) for dilation. After an appropriate time has elapsed, quickly place the mouse on the mouse stage and place the stage on chin rest of slit lamp. Turn on slit lamp to the lowest light brightness and check the degree of pupillary dilation. If pupil is not adequately dilated (approximately 2.5-3 mm), return mouse to animal warmer and wait. Alternatively, administer another drop of tropicamide. Once eye is sufficiently dilated, proceed to laser procedure. Adjust the placement of mouse on the mouse-stage, so that it is ideally positioned for visualization of optic nerve. Orient the mouse on its holder so it lies horizontally, perpendicular to slit lamp beam, with the head at one side and tail at the other. Slightly turn the mouse so it is at an approximately 170° angle with the head closer to laser operator. After the mouse is ideally positioned, place one drop of artificial tear solution on a 25 mm×25 mm glass coverslip. Place one drop of artificial tear solution on the opposite eye of the mouse—this will ensure the eye is hydrated and help delay cataract formation. Hold corner of coverslip between thumb and pointer fingers; position so that the glass is squeezed between tips of both fingers. Gently wrap the remaining three fingers around the animal's body for support and hand stabilization. Position hand so that the glass coverslip can be easily placed on the mouse's eye. Once stable position is obtained, carefully press glass coverslip (with drop of artificial tear still adhered) onto the mouse's eye. Make sure the coverslip is positioned as perpendicular as possible to the laser beam in order to prevent laser beam scatter or reflection. The coverslip acts as a contact lens to flatten the cornea. Look through slit lamp and with free hand toggle focus until retina can be visualized. The retina will have a light-yellow/red color depending on the location visualized, distinct, red vessels will be visible. Slowly and carefully manipulate mouse head and/or coverslip until visualizing the optic nerve. The optic nerve will be yellow in color with multiple vessels radiating from it. Once the operator has confirmed visualization of optic nerve, turn on laser focusing beam. Once the laser beam has been turned on, maneuver the laser focusing beam to desired position (approximately 1 disc diameter from the optic nerve). Focus laser beam on the RPE of the eye fundus. Proper focus is achieved by having the sharpest and clearest laser beam. If the aiming beam looks oval or out of focus, toggle slit lamp focus or re-position glass coverslip. Once the aiming beam is focused on RPE, initiate laser administration using the laser's foot trigger. Watch for the appearance of a bubble immediately after laser administration. The outline of the laser shot should be clear and not hazy in any way. Repeat previous 3 steps for all desired CNV positions (usually at 3, 6, 9, and 12 o'clock positions around optic nerve). Record in a notebook the location and result of each laser shot administration and result (Successful, hazy, hemorrhage, etc.) of each administered shot for the eye. Be sure to place the laser in stand-by mode when not in use. Repeat previous all steps for the mouse's other eye, if needed, using the opposite hand for stabilization and a new coverslip. After all desired laser shots are administered, turn off the laser and slit-lamp. Discard coverslip and place mouse on warmer for recovery from anesthesia. Acroscopically inspect the eye for any injury and place a drop of artificial tear solution to keep the eye hydrated and potentially prevent future cataract development. Once the mouse recovers from anesthesia, return to the cage.

### 4. Fluorescein Angiography

The eyes were fixed in 4% paraformaldehyde (Electron Microscopy Sciences, Hatfield, PA) for 2 hours after removal of the cornea and lens. Posterior eyecups of the RPE/choroid/sclera were dissected, and the vitreous was removed. Eyecups were incubated overnight at 4° C. with (AlexaFluor 647 or FITC)-conjugated Isolectin B4 (1:200, Invitrogen, Carlsbad, CA) to label invading choroidal vessels.

### 5. Expression of Angiogenic Factor by Western Blot

Cells were collected and lysed with PBS containing 1% Triton X-100. Equal amounts of proteins were loaded into the wells of the SDS-PAGE gel and transferred to nitrocellulose membrane filters for 2 hrs at 100V. The membranes were blocked with PBST (phosphate buffered saline with Tween 20) containing 1% BSA for 1 hr at room temperature and probed with anti-VEGF and anti-beta actin antibodies for 1 hr at room temperature. The membranes were washed three times with PBST, followed by incubation with a secondary antibody for 1 hr at room temperature. Following three washings with PBST, immune-reactive bands were detected. The data were quantified using Image J software.

### 6. Statistical Analysis

The Student's t-test was used for primary analysis. P-values were compared with mdm1−/− (AAV2+DX2). For recovery evaluation, P values were compared to WT. For full data, Levene's Homogeneity of Variance test, ANOVA tests, and post-hoc (Dunnett (T3), Tukey HSD) evaluation were performed with IBM SPSS statistics 23.

### Example 11. AAV2-DX2 Improves Retinal Degeneration in Sodium Iodate (SI)-Induced Rabbit RDD Model

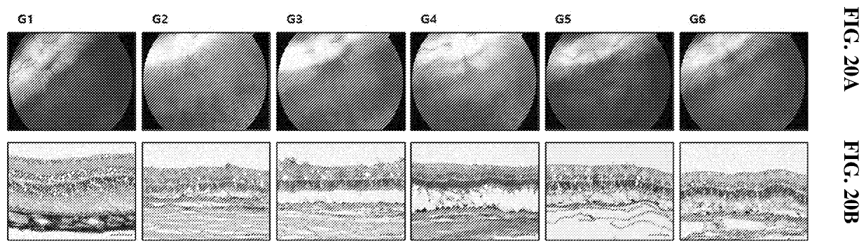

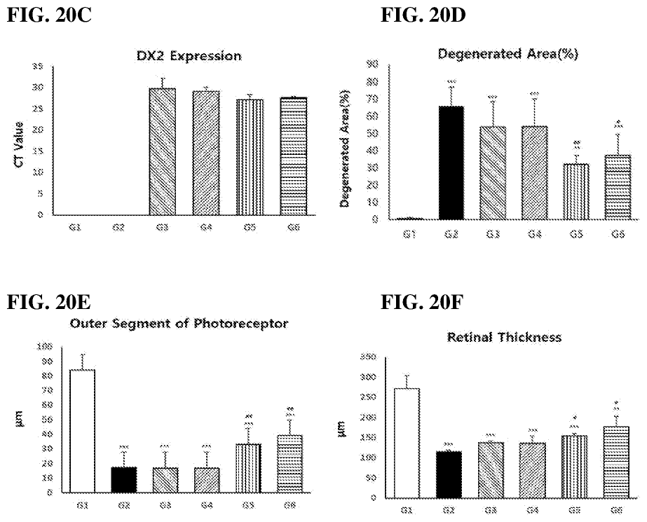

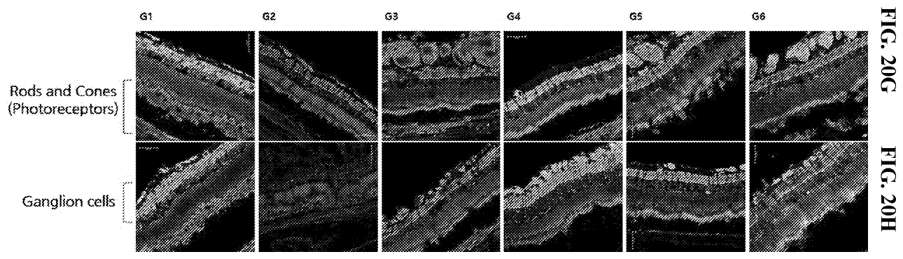

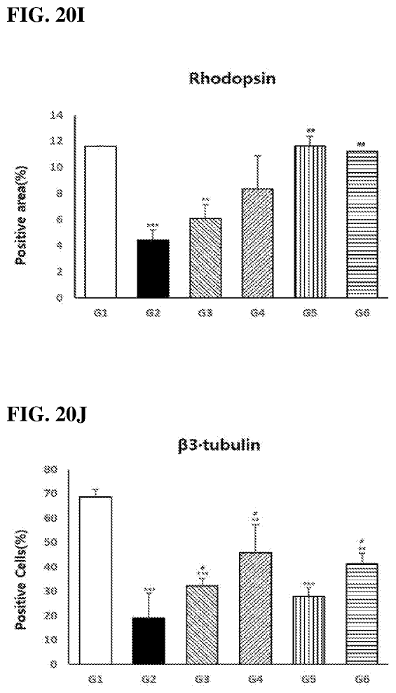

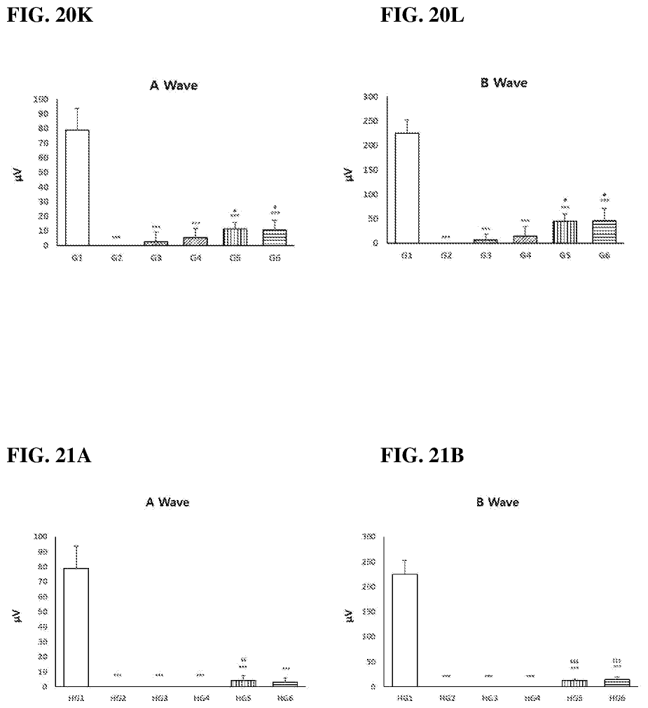

Sodium iodate (SI) is a widely used oxidant for generating retinal degeneration models by inducing the death of photoreceptor cells^{40 41} and retinal pigment epithelial cells (RPE). Sodium iodate solution was injected once intravenously at the dose of 30 mg/kg to generate RDD model⁴². At 21 days after 1st retinal Neurodegenerative induction, fundus images showed the RPE degeneration areas of sodium iodate induced group (G2) was significantly degenerated compared to normal control group (G1). RPE degenerated area (%) levels of test articles treated groups (G5 and G6) were significantly recovered compared to G2 (FIGS. 20A-20B).

In the Histological examination, the Retinal Pigment Epithelium (RPE) degenerated area (%) levels of Sodium-iodate inducted group (G2) was significantly degenerated compared to normal control group (G1), (p<0.001). RPE degenerated area (%) levels of test articles treated groups (G5 and G6) were significantly recovered compared to G2 (p<0.01, p<0.05) (FIG. 20D). Outer Segment of photoreceptor layer levels of sodium induced group (G2) was significantly reduced compared to normal control group (G1) (p<0.001) and the levels of test articles treated groups (G5 and G6) were recovered as compared to G2 (p<0.01) (FIG. 20E). Retinal thickness levels of sodium iodate induced group (G2) was significantly reduced compared to G1 (p<0.001). The levels of test articles treated groups (G5 and G6) were recovered compared to G2 (p<0.05) (FIG. 20F).

In the Immunohistochemistry analysis, Rhodopsin positive area (%) levels of G2 was significantly reduced compared to G1 (p<0.001, p<0.01). Rhodopsin positive area (%) levels of G5 and G6 were significantly recovered compared to G2 (p<0.01) (FIGS. 20G-20J). Ganglion positive cells (%) levels of G2 was significantly reduced compared to G1 (p<0.001). Ganglion positive cells (%) levels of G3, G4 and G6 were significantly recovered compared to G2 (p<0.05).

At 21 days after 1st Retinal Neurodegenerative induction, A wave and B wave amplitude levels of sodium iodate induced groups (G2) was significantly decreased compared to normal control group (G1) (p<0.001). A wave and B wave amplitude levels of test articles treated groups (G5 and G6) were significantly recovered compared to G2 (p<0.05) (FIGS. 20K-20L).

Finally, we generated a very severe RDD model to confirm that DX2 exerts its survival role and rescue the retinal function even in very severely destroyed retina. Sodium iodate solution was injected once intravenously at the dose of 60 mg/kg to generate a very severe RDD model. Koster C et al. (2022).

A statistically significant improvement of A wave and B wave amplitude levels compared to HG2 (severe RDD group) was observed in both DX2 treated HG Groups (i.e., 5*10¹⁰ VG dose of AAV2-DX2 injected via subretinal route or intravitreal route) (p<0.05). See Table 2 below.

TABLE 2

| | | Injection | Retinal degeneration | Treatment | |
|---|---|---|---|---|---|
| | | route | induction | dose | |
| HG1 | — | — | — | — | |
| HG2 | Vehicle | IVT | 5.60 | | |
| HG3 | AAV-DX2 | SR | NalO3 injection | 5*10⁹ | vg |
| HG4 | AAV-DX2 | IVT | (mg/kg) | 5*10⁹ | vg |
| HG5 | AAV-DX2 | SR | | 4*10¹⁰ | vg |
| HG6 | AAV-DX2 | IVT | | 4*10¹⁰ | vg |

FIGS. 21A-21B shows electroretinogram measurements of A wave (FIG. 21A) and B wave (FIG. 21B).

### Materials and Methods

### 1. Animal Experiments (Rabbits)

Male 52 Chinchilla rabbits (about 13 weeks, 2.5˜3.0 kg, Dream Bio Co., Ltd.) were used for the in vivo experiments. Environmental controls were set to maintain the following conditions: temperature range of 23±3° C., relative humidity range of 55±15%, ventilation of 10-20 air changes/hr, 150-300 Lux of luminous intensity and a 12-hr light/12-hr dark cycle. The animals were provided the lab animal diet purchased from Dream Bio manufactured by Cargill Agri Purina Inc., ad libitum. And water was given via polycarbonate water bottle, ad libitum. At the end of the experiment, after all surviving animals were anesthetized, euthanasia was performed, the right eyeballs were extracted and those of first half of each group were punctured on the center of cornea and fixed with 10% Neutral buffered formalin solution. Retina (choroid included) of the right eyeballs of second half of each group were sampled and put into micro tubes labeled with animal identification number, storing them in the deep freezer in which the temperature is kept below −70° C.

### 2. Sodium Iodate-Induced Retinal Neurodegenerative Disease Rabbits Model.

At the date of 1st induction (Day 0), sodium iodate (Sigma-Aldrich Co.) solution was injected once intravenously at the dose of 5 mg/kg to the animals, which were designated with degenerative eye diseases to be induced to, and at the date of 2nd induction (Day 14), sodium iodate solution was injected once intravenously at the dose of 30 mg/kg or 60 mg/kg in accordance with Settings for test group. In general, retinal degeneration studies are conducted with 25-30 mg/kg sodium iodate solution. However, retinal degeneration was induced with very high dose of sodium iodate solution (60 mg/kg) to test whether DX2 is effective in neuronal survival, even in situations where the optic nerve is destroyed and total visual loss occurred. Test articles were injected once at the date of administration (Day 5). In case of intravitreal injection, the animals were anesthetized, and the test articles were injected intravitreally into the right eyeball with the syringe equipped 31 gauge needle. In case of subretinal injection, the animal was anesthetized, and the test article were injected by trans-scleral route into the right eyeball with the syringe equipped 31-gauge needle.

Setting for test groups was in accordance with Table 3 below.

TABLE 3

| | | Injection | Retinal degeneration | Treatment | |
|---|---|---|---|---|---|
| | | route | induction | dose | |
| G1 | — | — | — | — | |
| G2 | Vehicle | IVT | 5.30 | — | |
| G3 | AAV-DX2 | SR | NaIO3 injection | 5*10⁹ | vg |
| G4 | AAV-DX2 | IVT | (mg/kg) | 5*10⁹ | vg |
| G5 | AAV-DX2 | SR | | 4*10¹⁰ | vg |
| G6 | AAV-DX2 | IVT | | 4*10¹⁰ | vg |

G1: Normal control.
G2: Vehicle control.
I.Vt: Intravitreal injection.
S.R: Subretinal injection.
vg: viral genome.

### 3. Fundus Photography

Before degenerative eye diseases induction and end of experiment, after applying eye drops (Mydriacyl oph soln, 1%) to both eyeballs, the animal was anesthetized, the fundus images were photographed with fundus camera (TRC-50IX, TOPCON, Japan). Retinal pigment epithelium (RPE) degenerated area in fundus images were analyzed using Image J (NIH, Bethesda, MD). Degenerated area (%)=Degenerated area/Total area.

### 4. Electrophysiological Function Evaluation

Before the end of the experiment, the right eye of all surviving animals were subjected to electroretinogram (ERG). After the animals were anesthetized, they be acclimatized in a dark room for 60 minutes, and the ERG measurement was carried out with ERG equipment (HMs-ERG, Ocuscience, USA). ERG was measured in a dark room with the flashlight, and A wave and B wave were evaluated by comparing amplitudes with normal control.

### 5. Histopathological Examination

After fixed ocular tissues were subjected to general tissue treatment such as cutting, dehydration, paraffin embedding, and dissection to produce specimens for histopathological examination, Hematoxylin & Eosin staining were carried out, the thickness of retinal Pigment Epithelium (RPE), Outer Segment of photoreceptor, Outer Nuclear Layer, Outer Plexiform Layer, and whole retina be measured using an optical microscope (Olympus BX53, Japan).

### 6. Expression of DX2 by RT-PCR

Total RNA was isolated from rabbits Retina (choroid included) using the RNA isolation Geneall hybrid-R Kit (GeneAll Biotechnology Co., Ltd., Korea) and reverse transcribed with the cDNA synthesis Kit (Toyobo, Japan). The complementary DNAs were then used for real time PCR using the qPCRBIO SyGreen Blue Mix (PCR Biosystems Ltd., UK). Amplification, detection, and data analysis were performed with a CFX96 touch (BioRad, USA).

### 7. Statistical Analysis

The results of this study were assumed to be normally distributed and analyzed by parametric multiple comparison procedures or non-parametric multiple comparison procedures. When one-way analysis of variance (ANOVA) results was significant, Dunnett's multiple comparison procedures were used as a post-hoc analysis.

Statistical analysis was performed using Prism 7.04 (GraphPad Software Inc., San Diego, CA, USA) and statistical significance was set at a P-value of less than 0.05.

### Example 12. AAV2-DX2 Show Preventive Potency of DX2 in the Laser-Induced Choroidal Neovascularization (CNV) Rabbit Model

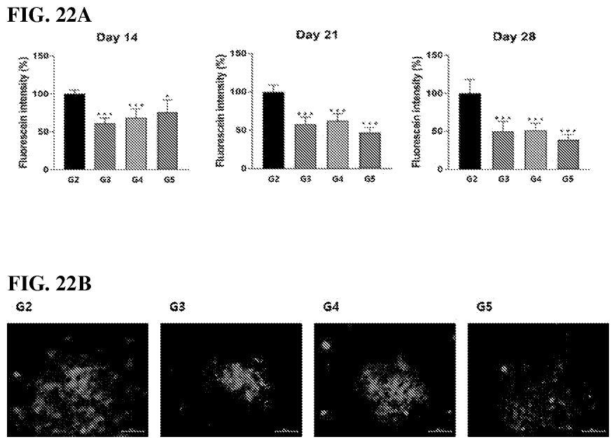

Laser-induced choroidal neovascularization (CNV) was conducted to generate retinal degenerative disease (RDD) rabbit model using Male 15 Chinchilla rabbits. As shown in FIG. 22A, as a result of choroidal neovascularization (CNV) area analysis, the fluorescein intensity levels G3 (SR), G4 (IVT), and positive control (G5) were significantly decreased compared to G2 (p<0.001, p<0.01 or p<0.05) at 14,21.28 days after inducing CNV.

FIG. 22B shows the analysis of CNV area using Isolectin B4 staining, the CNV area levels of all test article treated groups (G3, G4) and positive control (G5) values tends to decrease but did not reach statistical significance.

### Example. 13. AAV2-DX2 Did not Induce any Histopathological Abnormalities or Neoplastic Pattern in Whole Body Organs

A whole-body histopathological analysis was performed to test the potential toxicity of any tumorgenicity of AAV2-DX2. As summarized in Table 4, AAV2-DX2 administered animals did not show any histopathological abnormalities or neoplastic pattern in the kidney, liver, spleen, brain, lymph node, lung etc. The survival was not affected compared to control (G1) group.

TABLE 4
Organs Histopathological analysis
HISTOPATHOLOGICAL FINDINGS

| | | | | |
|---|---|---|---|---|
| ORGANS | G2 | G3 | G4 | G5 |

| | |
|---|---|
| BRAIN | No abnormality detected |
| TESTIS | No abnormality detected |
| KIDNEY-L | No abnormality detected |
| KIDNEY-R | No abnormality detected |
| LIVER | No abnormality detected |
| LUNG | No abnormality detected |
| SPINAL CORD | No abnormality detected |
| SPLEEN | No abnormality detected |
| STOMACH | No abnormality detected |
| HEART | No abnormality detected |
| THYMUS | No abnormality detected |
| PANCREAS | No abnormality detected |
| MESENTERIC LYMPH NODE | No abnormality detected |
| LEFT EYE (No laser induction) | No abnormality detected |

### Example 14. Preliminary Distribution Study to Confirm the Injected Tissue Neuronal Cell Specific DX2 Expression Via AAV2-DX2-miR-3 PT

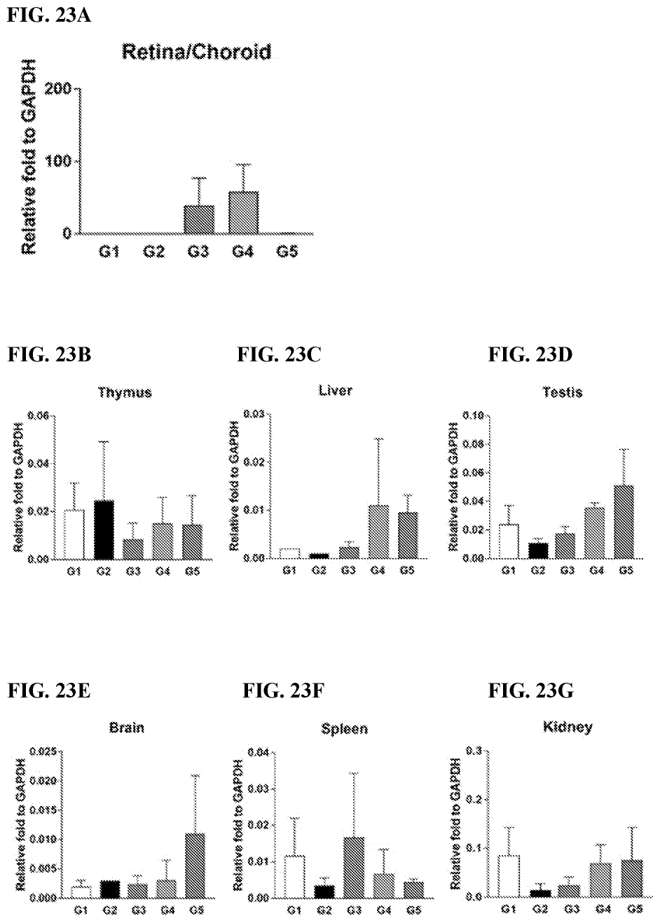

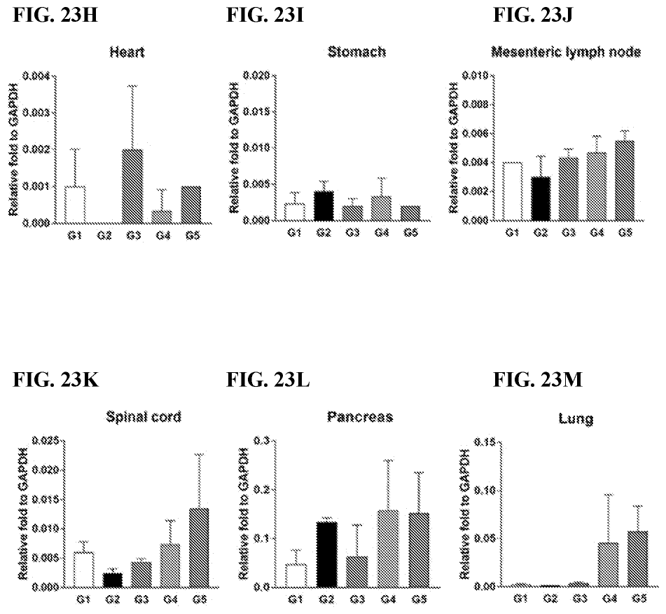

To observe the biodistribution pattern of DX2 using miR-3P T VECTOR system, the animals were sacrificed and expression of DX2 was analyzed in thymus, liver, testis, brain, spleen, kidney, heart, stomach, mesenteric lymph node, spinal cord, pancreas, lung using qPCR (FIGS. 23B-23M). qPCR analysis confirmed that the expression of DX2 was observed only in the injected tissue area (FIG. 23A).

### Materials and Methods

### 1. Animal Experiments (Rabbits)

Male 15 Chinchilla rabbits (about 13 weeks, 2.5˜3.0 kg, Dream Bio Co., Ltd.) were used for the in vivo experiments. Environmental controls were set to maintain the following conditions: temperature range of 23±3° C., relative humidity range of 55±15%, ventilation of 10-20 air changes/hr, 150-300 Lux of luminous intensity and a 12-hr light/12-hr dark cycle. The animals were provided the lab animal diet purchased from Dream Bio manufactured by Cargill Agri Purina Inc., ad libitum. And water was given via polycarbonate water bottle, ad libitum. At the end of the experiment, after all surviving animals were anesthetized, euthanasia was performed, the right eyeballs were extracted and those of first half of each group were punctured on the center of cornea and fixed with 10% Neutral buffered formalin solution. Retina (choroid included) of the right eyeballs of second half of each group were sampled and put into micro tubes labeled with animal identification number, storing them in the deep freezer in which the temperature is kept below-70° C. In case of intravitreal injection, the animal was anesthetized, and the test article was injected intravitreally into the right eyeball with the syringe equipped 31-gauge needle.

In case of subretinal injection, the animal was anesthetized, and the test article was injected by trans-scleral route into the right eyeball with the syringe equipped 31-gauge needle. After anesthetizing rabbits, whole body perfusion was carried out using sterile saline to the rabbits of G1 and the rabbits labeled with preceding number in each group, and the organs written below was extracted and stored in a deep freezer set below −70° C. before analysis.

Extracted organs: Brain, Testis, Kidney, Liver, Lung, Spinal cord, Spleen, Stomach, Heart, Thymus, Pancreas, Lymph node and retina/choroid of right eyes (of both eyes of G1).

At the end of experiment, after rabbits labeled with succeeding number were euthanatized after anesthesia, right eyes were extracted and the center of cornea was punctured with a needle and fixed with 10% neutral buffered formalin. Organs written below was extracted and fixed.

Extracted organs: Brain, Testis, Kidney, Liver, Lung, Spinal cord, Spleen, Stomach, Heart, Thymus, Pancreas and Lymph node.

Setting for test groups was in accordance with Table 5 below.

TABLE 5

| | | Disease Induction | Injection route | Treatment dose | |
|---|---|---|---|---|---|
| G1 | Vehicle | N | — | — | |
| G2 | Vehicle | Y | I.VT | — | |
| G3 | AAV-DX2 | Y | SR | 8*10¹⁰ | vg |
| G4 | AAV-DX2 | Y | I.VT | 8*10¹⁰ | vg |
| G5 | Eylea ® | Y | I.VT | 2000 | μg/eye |

G1: Normal control.
G2: Vehicle control.
I.Vt: Intravitreal injection.
S.R: Subretinal injection.
vg: viral genome.

### Rabbit Laser-Induced CNV Model Protocol

After applying eye drops (Mydriacyl oph soln, 1%) to right eyeball, the animal was anesthetized. The eyes were irradiated by laser (Elite, Lumenis, USA) at 532 nm, power 150 mW, duration 0.1 sec condition, followed by six spots generated around the optic nerve at about 6 o'clock.

### Fluorescein Angiography

After applying eye drops (Mydriacyl oph soln, 1%) to the right eye, the animal was anesthetized on Day 0, 7, 14, 21 and 28. And 1 mL of fluorescein sodium salt solution (2%) was injected by the vein to take the images using fundus camera (TRC-50IX, TOPCON, Japan) within 2 minutes.

Retinal CNV area and efficacy evaluation were performed using retinal fluorescent fundus photography. The Image analysis was performed using ImageJ software (NIH, Bethesda, MD) to verify the fluorescence intensity of the irradiation site.

Fluorescein intensity (%) was calculated as follows;

$$Fluorescein intensity ( \% ) = ( Fluorescein intensity value - average of background {value ( G 1 )}^{1 )} ) / average of fluorescein intensity value of G 2$$

Fluorescein intensity of retina without CNV spots were measured and used as background value.

### Histopathological Analysis

After fixed tissues were subjected to general tissue treatment such as cutting, dehydration, paraffin embedding, and dissection to produce specimens for histopathological examination, Hematoxylin & Eosin staining was carried out, histopathological changes were observed using an optical microscope (Olympus BX53, Japan). Isolectin B4 staining using choroidal flat mount method with fixed eye tissues was carried out to analyze CNV area.

### Expression of DX2 by RT-PCR

Total RNA was isolated from rabbit ocular tissues and each organs using the RNA isolation Geneall hybrid-R Kit (GeneAll Biotechnology Co., Ltd., Korea) and reverse transcribed with the cDNA synthesis Kit (Toyobo, Japan).

The complementary DNAs were then used for real time PCR using the qPCRBIO SyGreen Blue Mix (PCR Biosystems Ltd., UK). Amplification, detection, and data analysis were performed with a CFX96 touch (BioRad, USA). RT-PCR analysis of target gene using extracted organs and ocular tissues were carried out to confirm the quantity of target gene expression compared to GAPDH expression.

For the results of this study, the normality of the data was assumed, and the significance was verified between test groups using parametric One-way ANOVA. In case of significance, a post hoc test was performed using Dunnett's multiple comparison test. Statistical analysis was performed using Prism 7.04 (GraphPad Software Inc., San Diego, CA, USA), and was determined to be statistically significant when the p-value was less than 0.05.

### CONCLUSION

In the present study, the following have been demonstrated:) 1) the survival effect of DX2 on retinal degeneration; (2) utilization of DX2 as a treatment for retinal degenerative disease; the anti-apoptotic efficacy of DX2 delivered by adeno-associated virus (AAV) into the retina of mice and rabbit via SR and IV injection. Thus, the efficacy of gene therapy utilizing the survival gene DX2 as a potential option for treatment of Retinal degenerative disease was also confirmed.

### REFERENCES

1. Talib M, van Schooneveld M J, Van Cauwenbergh C, et al. The Spectrum of Structural and Functional Abnormalities in Female Carriers of Pathogenic Variants in the RPGR Gene. Invest Ophthalmol Vis Sci. Aug. 1, 2018; 59(10):4123-4133. doi:10.1167/iovs.17-23453.
2. Bennett J. Taking Stock of Retinal Gene Therapy: Looking Back and Moving Forward. Mol Ther. May 3 2017; 25(5):1076-1094. doi:10.1016/j.ymthe.2017.03.008.
3. Boye S E, Boye S L, Lewin A S, Hauswirth W W. A comprehensive review of retinal gene therapy. Mol Ther. March 2013; 21(3):509-19. doi:10.1038/mt.2012.280.
4. Sahel J A, Marazova K, Audo I. Clinical characteristics and current therapies for inherited retinal degenerations. Cold Spring Harb Perspect Med. Oct. 16 2014; 5(2):a017111. doi:10.1101/cshperspect.a017111.
5. Vandenberghe L H. What Is Next for Retinal Gene Therapy? Cold Spring Harb Perspect Med. Apr. 15 2015; 5(10) doi:10.1101/cshperspect.a017442.
6. Megaw R D, Soares D C, Wright A F. RPGR: Its role in photoreceptor physiology, human disease, and future therapies. Exp Eye Res. September 2015; 138:32-41. doi:10.1016/j.exer.2015.06.007.
7. Russell S, Bennett J, Wellman J A, et al. Efficacy and safety of voretigene neparvovec (AAV2-hRPE65v2) in patients with RPE65-mediated inherited retinal dystrophy: a randomised, controlled, open-label, phase 3 trial. Lancet. Aug. 26 2017; 390(10097):849-860. doi:10.1016/S0140-6736(17)31868-8.
8. Deng W T ea. Correction to: Stability and Safety of an AAV Vector for Treating RPGR-ORF15X-Linked Retinitis Pigmentosa by Deng, W.-T. et al. Hum Gene Ther 2015; 26(9):593-602. DOI: 10.1089/hum.2015.035. Hum Gene Ther. February 2021; 32(3-4):232-233. doi:10.1089/hum.2015.035.correx.
9. Pawlyk B S, Bulgakov O V, Sun X, et al. Photoreceptor rescue by an abbreviated human RPGR gene in a murine model of X-linked retinitis pigmentosa. Gene Ther. February 2016; 23(2):196-204. doi:10.1038/gt.2015.93.
10. Sun X, Park J H, Gumerson J, et al. Loss of RPGR glutamylation underlies the pathogenic mechanism of retinal dystrophy caused by TTLL5 mutations. Proc Natl Acad Sci USA. May 24 2016; 113(21): E2925-34. doi:10.1073/pnas.1523201113.
11. Wu Z, Hiriyanna S, Qian H, et al. A long-term efficacy study of gene replacement therapy for RPGR-associated retinal degeneration. Hum Mol Genet. Jul. 15 2015; 24(14):3956-70. doi:10.1093/hmg/ddv134.
12. Jin Z B, Gao M L, Deng W L, et al. Stemming retinal regeneration with pluripotent stem cells. Prog Retin Eye Res. March 2019; 69:38-56. doi:10.1016/j.preteyeres.2018.11.003.
13. Soldatov V O, Kukharsky M S, Belykh A E, Sobolev A M, Deykin A V. Retinal Damage in Amyotrophic Lateral Sclerosis: Underlying Mechanisms. Eye Brain. 2021; 13:131-146. doi:10.2147/EB.S299423.
14. Kim J H, Han J M, Kim S. Protein-protein interactions and multi-component complexes of aminoacyl-tRNA synthetases. Top Curr Chem. 2014; 344:119-44. doi:10.1007/128_2013_479.
15. Choi J W, Kim D G, Lee A E, et al. Cancer-associated splicing variant of tumor suppressor AIMP2/p38: pathological implication in tumorigenesis. PLOS Genet. March 2011; 7(3):e1001351. doi:10.1371/journal.pgen.1001351.
16. Ko H S, von Coelln R, Sriram S R, et al. Accumulation of the authentic parkin substrate aminoacyl-tRNA synthetase cofactor, p38/JTV-1, leads to catecholaminergic cell death. J Neurosci. Aug. 31, 2005; 25(35):7968-78. doi:10.1523/JNEUROSCI.2172-05.2005.
17. Lee Y, Karuppagounder S S, Shin J H, et al. Parthanatos mediates AIMP2-activated age-dependent dopaminergic neuronal loss. Nat Neurosci. October 2013; 16(10):1392-400. doi:10.1038/nn.3500.
18. de Murcia J M, Niedergang C, Trucco C, et al. Requirement of poly (ADP-ribose) polymerase in recovery from DNA damage in mice and in cells. Proc Natl Acad Sci USA. Jul. 8 1997; 94(14):7303-7. doi:10.1073/pnas.94.14.7303.
19. Hong S J, Dawson T M, Dawson V L. Nuclear and mitochondrial conversations in cell death: PARP-1 and AIF signaling. Trends Pharmacol Sci. May 2004; 25(5):259-64. doi:10.1016/j.tips.2004.03.005.
20. Choi J W, Kim D G, Park M C, et al. AIMP2 promotes TNFalpha-dependent apoptosis via ubiquitin-mediated degradation of TRAF2. J Cell Sci. Aug. 1, 2009; 122 (Pt 15):2710-5. doi:10.1242/jcs.049767.
21. Corti O, Hampe C, Koutnikova H, et al. The p38 subunit of the aminoacyl-tRNA synthetase complex is a Parkin substrate: linking protein biosynthesis and neurodegeneration. Hum Mol Genet. Jun. 15 2003; 12(12):1427-37. doi:10.1093/hmg/ddg159.
22. Ko H S, Lee Y, Shin J H, et al. Phosphorylation by the c-Abl protein tyrosine kinase inhibits parkin's ubiquitination and protective function. Proc Natl Acad Sci USA. Sep. 21 2010; 107(38):16691-6. doi:10.1073/pnas.1006083107.
23. Narne P, Pandey V, Simhadri P K, Phanithi P B. Poly (ADP-ribose) polymerase-1 hyperactivation in neurodegenerative diseases: The death knell tolls for neurons. Semin Cell Dev Biol. March 2017; 63:154-166. doi:10.1016/j.semcdb.2016.11.007.
24. Mao K, Zhang G. The role of PARP1 in neurodegenerative diseases and aging. FEBS J. April 2022; 289(8):2013-2024. doi:10.1111/febs.15716.
25. Szabo C, Zingarelli B, O'Connor M, Salzman A L. DNA strand breakage, activation of poly (ADP-ribose) synthetase, and cellular energy depletion are involved in the cytotoxicity of macrophages and smooth muscle cells exposed to peroxynitrite. Proc Natl Acad Sci USA. Mar. 5 1996; 93(5):1753-8. doi:10.1073/pnas.93.5.1753.
26. Virag L, Salzman A L, Szabo C. Poly (ADP-ribose) synthetase activation mediates mitochondrial injury during oxidant-induced cell death. J Immunol. Oct. 1, 1998; 161(7):3753-9.
27. Sahaboglu A, Sharif A, Feng L, Secer E, Zrenner E, Paquet-Durand F. Temporal progression of PARP activity in the Prph2 mutant rd2 mouse: Neuroprotective effects of the PARP inhibitor PJ34. PLOS One. 2017; 12(7):e0181374. doi:10.1371/journal.pone. 0181374.
28. Olivares-Gonzalez L, Velasco S, Campillo I, Rodrigo R. Retinal Inflammation, Cell Death and Inherited Retinal Dystrophies. Int J Mol Sci. Feb. 20 2021; 22(4) doi:10.3390/ijms22042096
29. Streit W J, Mrak R E, Griffin W S. Microglia and neuroinflammation: a pathological perspective. J Neuroinflammation. Jul. 30 2004; 1(1):14. doi:10.1186/1742-2094-1-14.
30. Karlstetter M, Scholz R, Rutar M, Wong W T, Provis J M, Langmann T. Retinal microglia: just bystander or target for therapy? Prog Retin Eye Res. March 2015; 45:30-57. doi:10.1016/j.preteyeres.2014.11.004.
31. Murakami Y, Ishikawa K, Nakao S, Sonoda K H. Innate immune response in retinal homeostasis and inflammatory disorders. Prog Retin Eye Res. January 2020; 74:100778. doi:10.1016/j.preteyeres.2019.100778.
32. Rodrigues E B, Farah M E, Maia M, et al. Therapeutic monoclonal antibodies in ophthalmology. Prog Retin Eye Res. March 2009; 28(2):117-44. doi:10.1016/j.preteyeres.2008.11.005.
33. Choi J W, Lee J W, Kim J K, et al. Splicing variant of AIMP2 as an effective target against chemoresistant ovarian cancer. J Mol Cell Biol. June 2012; 4(3):164-73. doi:10.1093/jmcb/mjs018.
34. Christine C W, Starr P A, Larson P S, et al. Safety and tolerability of putaminal AADC gene therapy for Parkinson disease. Neurology. Nov. 17 2009; 73(20):1662-9. doi:10.1212/WNL.0b013e3181c29356.
35. Eberling J L, Jagust W J, Christine C W, et al. Results from a phase I safety trial of hAADC gene therapy for Parkinson disease. Neurology. May 20 2008; 70(21):1980-3. doi:10.1212/01.wnl.0000312381.29287.ff.
36. Castle M J, Baltanas F C, Kovacs I, Nagahara A H, Barba D, Tuszynski M H. Postmortem Analysis in a Clinical Trial of AAV2-NGF Gene Therapy for Alzheimer's Disease Identifies a Need for Improved Vector Delivery. Hum Gene Ther. April 2020; 31(7-8):415-422. doi:10.1089/hum.2019.367.
37. Wang D, Tai PWL, Gao G. Adeno-associated virus vector as a platform for gene therapy delivery. Nat Rev Drug Discov. May 2019; 18(5):358-378. doi:10.1038/s41573-019-0012-9.
38. Yan X, Li Y, Yu H, et al. Epigallocatechin-3-gallate inhibits H (2) O (2)-induced apoptosis in Mouse Vascular Smooth Muscle Cells via 67 kD Laminin Receptor. Sci Rep. Aug. 10 2017; 7(1):7774. doi:10.1038/s41598-017-08301-6.
39. Chang B, Mandal M N, Chavali V R, et al. Age-related retinal degeneration (arrd2) in a novel mouse model due to a nonsense mutation in the Mdml gene. Hum Mol Genet. Dec. 15 2008; 17(24):3929-41. doi:10.1093/hmg/ddn295.
40. Zhou; R. Sodium iodate-induced retina and choroid damage model in rabbits to test efficacy of RPE auto-transplants. Vol. 57, 2253. https://iovs.arvojournals.org/article.aspx?articleid=2560738&resultClick=1.
41. Ahn S M, Ahn J, Cha S, et al. The effects of intravitreal sodium iodate injection on retinal degeneration following vitrectomy in rabbits. Sci Rep. Oct. 30 2019; 9(1):15696. doi:10.1038/s41598-019-52172-y.
42. Koster C, van den Hurk K T, Ten Brink J B, et al. Sodium-Iodate Injection Can Replicate Retinal Degenerative Disease Stages in Pigmented Mice and Rats: Non-Invasive Follow-Up Using OCT and ERG. Int J Mol Sci. Mar. 8 2022; 23(6) doi:10.3390/ijms23062918.
43. Telegina, D. V., O. S. Kozhevnikova, and N. G. Kolosova. “Molecular mechanisms of cell death in retina during development of age-related macular degeneration.” Advances in Gerontology 7.1 (2017): 17-24.
44. Hernández-Zimbrón, Luis Fernando, et al. “Age-related macular degeneration: new paradigms for treatment and management of AMD.” Oxidative medicine and cellular longevity 2018 (2018).
45. Du, Hongjun, et al. “JNK inhibition reduces apoptosis and neovascularization in a murine model of age-related macular degeneration.” Proceedings of the National Academy of Sciences 110.6 (2013):2377-2382.
46. Brown et al., Endogenous microRNA regulation suppresses transgene expression in hematopoietic lineages and enables stable gene transfer, Nature Med. 12:585-591 (2006).
47. Brown et al., Endogenous microRNA can broadly exploited to regulate transggene expression according to tissue, lineage and differentiation state, Nature Biotech. 25:12457-1467 (2007).
48. KR 10-1067816 (2011).
49. US2019/0298858 A1.

The foregoing description of the specific embodiments will so fully reveal the general nature of the invention that others can, by applying knowledge within the skill of the art, readily modify and/or adapt for various applications, without departing from the general concept of the invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments but should be defined only in accordance with the following claims and their equivalents.

All of the various aspects, embodiments, and options described herein can be combined in any and all variations.

All publications, patents, and patent applications mentioned in this specification are herein incorporated by reference to the same extent as if each individual publication, patent, or patent application was specifically and individually indicated to be incorporated by reference.

What is claimed is:

1. A method of treating a retinal degenerative disease in a subject in need thereof, comprising administering to the subject a pharmaceutically effective amount of a recombinant vector comprising an exon 2-deleted AIMP2 variant (AIMP2-DX2) gene.

2. The method of claim 1, wherein the retinal degenerative disease is retinitis pigmentosa, Leber's congenital amaurosis, Cone-rod dystrophy, glaucoma, or diabetic retinopathy.

3. The method of claim 1 or 2, wherein the retinal degenerative disease precedes or is accompanied by Parkinson's disease, Alzheimers's disease, or amyotrophic lateral sclerosis.

4. The method of any one of claims 1-3, wherein the retinal degenerative disease is not age-related macular disease.

5. The method of any one of claims 1-4, wherein the vector further comprises an miR-142 target sequence.

6. The method of any one of claims 1-5, wherein the vector further comprises a promoter operably linked to the AIMP2-DX2.

7. The method of claim 6, wherein the promoter is a Retrovirus (LTR) promoter, cytomegalovirus (CMV) promoter, Rous sarcoma virus (RSV) promoter, MT promoter, EF-1 alpha promoter, UB6 promoter, chicken beta-actin promoter, CAG promoter, RPE65 promoter, Synapsin promoter, MeCP2 promoter, CaMKII promoter, Hb9 promoter, or opsin promoter.

8. The method of any one of claims 5-7, wherein the miR-142 target sequence is 3′ to the AIMP2-DX2 gene.

9. The method of any one of claims 1-8, wherein the AIMP2-DX2 gene comprises a nucleotide sequence encoding an amino acid sequence that is at least 90% identical to SEQ ID NO: 2, 13, 14, 15, 16, 17, 18, 19, or 20.

10. The method of claim 9, wherein the AIMP2-DX2 gene comprises a nucleotide sequence encoding an amino acid sequence of SEQ ID NO:2, 13, 14, 15, 16, 17, 18, 19, or 20.

11. The method of any one of claims 1-10, wherein the AIMP2-DX2 gene does not have an exon comprising a nucleotide sequence encoding an amino acid sequence that is at least 90% identical to SEQ ID NO:10 or 11.

12. The method of any one of claims 1-11, wherein the AIMP2-DX2 gene does not have an exon comprising a nucleotide sequence encoding an amino acid sequence of SEQ ID NO: 10 or 11.

13. The method of any one of claims 5-12, wherein the miR-142 target sequence comprises ACACTA.

14. The method of claim 5-12, wherein the miR-142 target sequence comprises ACACTA and 1-17 additional contiguous nucleotides of SEQ ID NO:5.

15. The method of any one of claims 5-12, wherein the miR-142 target sequence comprises a nucleotide sequence at least 50% identical to a nucleotide sequence of SEQ ID NO:5 (TCCATAAAGTAGGAAACACTACA).

16. The method of claim 15, wherein the miR-142 target sequence comprises a nucleotide sequence of SEQ ID NO:5.

17. The method of any one of claims 5-12, wherein the miR-142 target sequence comprises ACTTTA.

18. The method of claim 5-12, wherein the miR-142 target sequence comprises ACTTTA and 1-15 additional contiguous nucleotides of SEQ ID NO:7.

19. The method of any one of claims 5-12, wherein the miR-142 target sequence comprises a nucleotide sequence at least 50% identical to a nucleotide sequence of SEQ ID NO:7 (AGTAGTGCTTTCTACTTTATG).

20. The method of claim 19, wherein the miR-142 target sequence comprises a nucleotide sequence of SEQ ID NO:7.

21. The method of any one of claims 5-20, wherein the miR-142 target sequence is repeated 2-10 times.

22. The method of any one of claims 1-21, wherein the vector is a viral vector.

23. The method of claim 22, wherein the viral vector is an adenovirus, adeno-associated virus, lentivirus, retrovirus, human immunodeficiency virus (HIV), murine leukemia virus (MLV), avian sarcoma/leukosis (ASLV), spleen necrosis virus (SNV), Rous sarcoma virus (RSV), mouse mammary tumor virus (MMTV), vaccinia virus, or Herpes simplex virus vector.

24. The method of any one of claims 1-23, wherein the recombinant vector is administered topically to, by intravitreal injection to, by subconjunctival injection to, or into a subretinal space of the subject.

25. The method of any one of claims 1-24, further comprising administering to the subject an additional therapeutic agent.

26. The method of claim 25, wherein the additional therapeutic agent is ranibizumab, aflibercept, or bevacizumab.


SEQUENCE LISTING (25 sequences)

SEQ ID NO: 1 - DNA, 756 bp, synthetic construct
  misc_feature 1..756: note = AIMP2-DX2
  source 1..756: mol_type = other DNA
atgccgatgtaccaggtaaagccctatcacgggggcggcgcgcctctccgtgtggagcttcccacctgcatgtaccggctccccaacgtgcacggcaggagctacggcccagcgccgggcgctggccacgtgcaggattacggggcgctgaaagacatcgtgatcaacgcaaacccggcctcccctcccctctccctgcttgtgctgcacaggctgctctgtgagcacttcagggtcctgtccacggtgcacacgcactcctcggtcaagagcgtgcctgaaaaccttctcaagtgctttggagaacagaataaaaaacagccccgccaagactatcagctgggattcactttaatttggaagaatgtgccgaagacgcagatgaaattcagcatccagacgatgtgccccatcgaaggcgaagggaacattgcacgtttcttgttctctctgtttggccagaagcataatgctgtcaacgcaacccttatagatagctgggtagatattgcgatttttcagttaaaagagggaagcagtaaagaaaaagccgctgttttccgctccatgaactctgctcttgggaagagcccttggctcgctgggaatgaactcaccgtagcagacgtggtgctgtggtctgtactccagcagatcggaggctgcagtgtgacagtgccagccaatgtgcagaggtggatgaggtcttgtgaaaacctggctccttttaacacggccctcaagctccttaagtga

SEQ ID NO: 2 - protein, 251 aa, synthetic construct
  REGION 1..251: note = AIMP2-DX2
MPMYQVKPYHGGGAPLRVELPTCMYRLPNVHGRSYGPAPGAGHVQDYGALKDIVINANPASPPLSLLVLHRLLCEHFRVLSTVHTHSSVKSVPENLLKCFGEQNKKQPRQDYQLGFTLIWKNVPKTQMKFSIQTMCPIEGEGNIARFLFSLFGQKHNAVNATLIDSWVDIAIFQLKEGSSKEKAAVFRSMNSALGKSPWLAGNELTVADVVLWSVLQQIGGCSVTVPANVQRWMRSCENLAPFNTALKLLK

SEQ ID NO: 3 - DNA, 936 bp, synthetic construct
  misc_feature 1..936: note = AIMP2
  source 1..936: mol_type = other DNA
atgccgatgtaccaggtaaagccctatcacgggggcggcgcgcctctccgtgtggagcttcccacctgcatgtaccggctccccaacgtgcacggcaggagctacggcccagcgccgggcgctggccacgtgcaggaagagtctaacctgtctctgcaagctcttgagtcccgccaagatgatattttaaaacgtctgtatgagttgaaagctgcagttgatggcctctccaagatgattcaaacaccagatgcagacttggatgtaaccaacataatccaagcggatgagcccacgactttaaccaccaatgcgctggacttgaattcagtgcttgggaaggattacggggcgctgaaagacatcgtgatcaacgcaaacccggcctcccctcccctctccctgcttgtgctgcacaggctgctctgtgagcacttcagggtcctgtccacggtgcacacgcactcctcggtcaagagcgtgcctgaaaaccttctcaagtgctttggagaacagaataaaaaacagccccgccaagactatcagctgggattcactttaatttggaagaatgtgccgaagacgcagatgaaattcagcatccagacgatgtgccccatcgaaggcgaagggaacattgcacgtttcttgttctctctgtttggccagaagcataatgctgtcaacgcaacccttatagatagctgggtagatattgcgatttttcagttaaaagagggaagcagtaaagaaaaagccgctgttttccgctccatgaactctgctcttgggaagagcccttggctcgctgggaatgaactcaccgtagcagacgtggtgctgtggtctgtactccagcagatcggaggctgcagtgtgacagtgccagccaatgtgcagaggtggatgaggtcttgtgaaaacctggctcctttt

SEQ ID NO: 4 - DNA, 207 bp, synthetic construct
  misc_feature 1..207: note = Exon 2 of AIMP2
  source 1..207: mol_type = other DNA
gaagagtctaacctgtctctgcaagctcttgagtcccgccaagatgatattttaaaacgtctgtatgagttgaaagctgcagttgatggcctctccaagatgattcaaacaccagatgcagacttggatgtaaccaacataatccaagcggatgagcccacgactttaaccaccaatgcgctggacttgaattcagtgcttgggaag

SEQ ID NO: 5 - DNA, 23 bp, synthetic construct
  misc_feature 1..23: note = miR-142-3p target sequence
  source 1..23: mol_type = other DNA
tccataaagtaggaaacactaca

SEQ ID NO: 6 - DNA, 132 bp, synthetic construct
  misc_feature 1..132: note = 4 repeats of miR-142-3p target sequence
  source 1..132: mol_type = other DNA
ccagaagcttgctagctccataaagtaggaaacactacatcactccataaagtaggaaacactacagatatctccataaagtaggaaacactacatcactccataaagtaggaaacactacaaagcttgtag

SEQ ID NO: 7 - DNA, 21 bp, synthetic construct
  misc_feature 1..21: note = miR-142-5p target sequence
  source 1..21: mol_type = other DNA
agtagtgctttctactttatg

SEQ ID NO: 8 - DNA, 24 bp, synthetic construct
  misc_feature 1..24: note = AIMP2 variant forward primer
  source 1..24: mol_type = other DNA
ctggccacgtgcaggattacgggg

SEQ ID NO: 9 - DNA, 20 bp, synthetic construct
  misc_feature 1..20: note = AIMP2 variant reverse primer
  source 1..20: mol_type = other DNA
aagtgaatcccagctgatag

SEQ ID NO: 10 - protein, 69 aa, synthetic construct
  REGION 1..69: note = Exon 2 of AIMP2
EESNLSLQALESRQDDILKRLYELKAAVDGLSKMIQTPDADLDVTNIIQADEPTTLTTNALDLNSVLGK

SEQ ID NO: 11 - protein, 36 aa, synthetic construct
  REGION 1..36: note = Exon 2 of AIMP2 variant
MIQTPDADLDVTNIIQADEPTTLTTNALDLNSVLGK

SEQ ID NO: 12 - protein, 320 aa, synthetic construct [sequence omitted]

SEQ ID NO: 13 - protein, 243 aa, synthetic construct
  REGION 1..243: note = AIMP-DX2 variant 1
MPMYQVKPYHGGGAPLRVELPTCMYRLPNVHGRSYGPAPGAGHVQDYGALKDIVINANPASPPLSLLVLHRLLCEHFRVLSTVHTHSSVKSVPENLLKCFGEQNKKQPRQDYQLGFTLIWKNVPKTQMKFSIQTMCPIEGEGNIARFLFSLFGQKHNAVNATLIDSWVDIAIFQLKEGSSKEKAAVFRSMNSALGKSPWLAGNELTVADVVLWSVLQQIGGCSVTVPANVQRWMRSCENLAPF

SEQ ID NO: 14 - protein, 244 aa, synthetic construct
  REGION 1..244: note = AIMP2-DX2 variant 2
MQMEGTAHVKICGQSQGGGLGTPRTVWLEHRQRTKLGEDYGALKDIVINANPASPPLSLLVLHRLLCEHFRVLSTVHTHSSVKSVPENLLKCFGEQNKKQPRQDYQLGFTLIWKNVPKTQMKFSIQTMCPIEGEGNIARFLFSLFGQKHNAVNATLIDSWVDIAIFQLKEGSSKEKAAVFRSMNSALGKSPWLAGNELTVADVVLWSVLQQIGGCSVTVPANVQRWMRSCENLAPFNTALKLLK

SEQ ID NO: 15 - protein, 207 aa, synthetic construct
  REGION 1..207: note = AIMP2-DX2 variant 3
MDYGALKDIVINANPASPPLSLLVLHRLLCEHFRVLSTVHTHSSVKSVPENLLKCFGEQNKKQPRQDYQLGFTLIWKNVPKTQMKFSIQTMCPIEGEGNIARFLFSLFGQKHNAVNATLIDSWVDIAIFQLKEGSSKEKAAVFRSMNSALGKSPWLAGNELTVADVVLWSVLQQIGGCSVTVPANVQRWMRSCENLAPFNTALKLLK

SEQ ID NO: 16 - protein, 222 aa, synthetic construct
  REGION 1..222: note = AIMP2-DX2 variant 4
MNSPAVNTLIQRSRHGDYGALKDIVINANPASPPLSLLVLHRLLCEHFRVLSTVHTHSSVKSVPENLLKCFGEQNKKQPRQDYQLGFTLIWKNVPKTQMKFSIQTMCPIEGEGNIARFLFSLFGQKHNAVNATLIDSWVDIAIFQLKEGSSKEKAAVFRSMNSALGKSPWLAGNELTVADVVLWSVLQQIGGCSVTVPANVQRWMRSCENLAPFNTALKLLK

SEQ ID NO: 17 - protein, 211 aa, synthetic construct
  REGION 1..211: note = AIMP2-DX2 variant 5
MPMYQDYGALKDIVINANPASPPLSLLVLHRLLCEHFRVLSTVHTHSSVKSVPENLLKCFGEQNKKQPRQDYQLGFTLIWKNVPKTQMKFSIQTMCPIEGEGNIARFLFSLFGQKHNAVNATLIDSWVDIAIFQLKEGSSKEKAAVFRSMNSALGKSPWLAGNELTVADVVLWSVLQQIGGCSVTVPANVQRWMRSCENLAPFNTALKLLK

SEQ ID NO: 18 - protein, 251 aa, synthetic construct; identical to SEQ ID NO: 2
  REGION 1..251: note = AIMP2-DX2 variant 6

SEQ ID NO: 19 - protein, 206 aa, synthetic construct
  REGION 1..206: note = AIMP2-DX2 variant 7
DYGALKDIVINANPASPPLSLLVLHRLLCEHFRVLSTVHTHSSVKSVPENLLKCFGEQNKKQPRQDYQLGFTLIWKNVPKTQMKFSIQTMCPIEGEGNIARFLFSLFGQKHNAVNATLIDSWVDIAIFQLKEGSSKEKAAVFRSMNSALGKSPWLAGNELTVADVVLWSVLQQIGGCSVTVPANVQRWMRSCENLAPFNTALKLLK

SEQ ID NO: 20 - protein, 198 aa, synthetic construct
  REGION 1..198: note = AIMP2-DX2 consensus sequence
DYGALKDIVINANPASPPLSLLVLHRLLCEHFRVLSTVHTHSSVKSVPENLLKCFGEQNKKQPRQDYQLGFTLIWKNVPKTQMKFSIQTMCPIEGEGNIARFLFSLFGQKHNAVNATLIDSWVDIAIFQLKEGSSKEKAAVFRSMNSALGKSPWLAGNELTVADVVLWSVLQQIGGCSVTVPANVQRWMRSCENLAPF

SEQ ID NO: 21 - DNA, 16 bp, synthetic construct
  misc_feature 1..16: note = Nhe1, Hind III, and Bmt1 sites in miR-142-3pT with 4 repeats
  source 1..16: mol_type = other DNA
ccagaagcttgctagc

SEQ ID NO: 22 - DNA, 10 bp, synthetic construct
  misc_feature 1..10: note = Hind H site in miR-142-3pT with 4 repeats
  source 1..10: mol_type = other DNA
aagcttgtag

SEQ ID NO: 23 - RNA, 23 nt, synthetic construct
  misc_feature 1..23: note = miR-142-3p
  source 1..23: mol_type = other RNA
tgtagtgtttcctactttatgga

SEQ ID NO: 24 - RNA, 21 nt, synthetic construct
  misc_feature 1..21: note = miR-142-5p
  source 1..21: mol_type = other RNA
cataaagtagaaagcactact

SEQ ID NO: 25 - DNA, 238 bp, synthetic construct
  misc_feature 1..238: note = miR-142-3pT target sequence mutant
  source 1..238: mol_type = other DNA
ccgctgcagtgtgacagtgccagccaatgtgcagaggtggatgaggtcttgtgaaaacctggctccttttaacacggccctcaagctccttaagtgaccagaagcttgctagctccataaagtaggaccactgcaatcactccataaagtaggaccactgcaagatatctccataaagtaggaccactgcaatcactccataaagtaggaccactgcaaaagcttgtagggatccgcc